(19) United States
(12) Patent Application Publication
(10) Pub. No.: US 2011/0319849 A1
(43) Pub. Date: Dec. 29, 2011

(54) ABSORBENT ARTICLE COMPRISING A SYNTHETIC POLYMER DERIVED FROM A RENEWABLE RESOURCE AND METHODS OF PRODUCING SAID ARTICLE

(76) Inventors: Dimitris Ioannis Collias, Mason, OH (US); Patti Jean Kellett, Cincinnati, OH (US); Janette Villalobos Lingoes, Cincinnati, OH (US)

(21) Appl. No.: 13/175,145

(22) Filed: Jul. 1, 2011

(51) Int. Cl. A61L 15/22; B01J 20/26

(52) U.S. Cl. 604/372; 502/402

(57) ABSTRACT

An element of an absorbent article is provided. The element has a bio-based content of at least about 50% based on the total weight of the element, and comprises a synthetic polymer derived from a renewable resource via a first intermediate compound selected from the group consisting of crotonic acid, propiolactone, ethylene oxide, i-propanol, butanol, butyric acid, propionic acid, 2-acetoxypropanoic acid, methyl 2-acetoxypropanoate, methyl lactate, ethyl lactate, polyhydroxybutyrate, and a polyhydroxyalkanoate comprising 3-hydroxypropionate monomers. An absorbent article comprising the element and a method of making an element for an absorbent article also are provided.

### FIELD OF INVENTION

The invention relates to an absorbent article which comprises synthetic polymeric materials derived from renewable resources, where the materials have specific performance characteristics making them particularly useful in said absorbent article.

### BACKGROUND OF THE INVENTION

The development of absorbent articles such as disposable diapers, adult incontinence pads and briefs, and catamenial products such as sanitary napkins, is the subject of substantial commercial interest. There is a great deal of art relating to the design of absorbent articles, the processes for manufacturing such articles, and the materials used in their construction. In particular, a great deal of effort has been spent in the development of materials exhibiting optimal performance characteristics for use in absorbent articles. Such materials include films, fibers, nonwovens, laminates, superabsorbent polymers, foams, elastomers, adhesives, and the like.

Most of the materials used in current commercial absorbent articles are derived from non-renewable resources, especially petroleum and natural gas. Typically, components such as the topsheet, backsheet, and cuffs are made from polyolefins such as polyethylene and polypropylene. These polymers are derived from olefinic monomers such as ethylene and propylene which are obtained directly from petroleum or natural gas via cracking and refining processes.

Propylene derived from petroleum is also used to make acrylic acid via a catalytic oxidation process. Acrylic acid derived from petroleum is the major feedstock used in the manufacture of modern superabsorbent polymers utilized in absorbent cores of current commercial absorbent articles.

Thus, the price and availability of the petroleum feedstock ultimately has a significant impact on the price of absorbent articles which utilize materials derived from petroleum. As the worldwide price of petroleum escalates, so does the price of absorbent articles.

Furthermore, many consumers display an aversion to purchasing products that are derived from petrochemicals. In some instances, consumers are hesitant to purchase products made from limited non-renewable resources such as petroleum and coal. Other consumers may have adverse perceptions about products derived from petrochemicals being “unnatural” or not environmentally friendly.

Certain alternative materials which are derived from non-petrochemical or renewable resources and are not acrylic acid-based superabsorbent materials have been disclosed for use in absorbent articles. For example, U.S. Pat. No. 5,889,072 to Chao describes a process for preparing a cross-linked polyaspartate superabsorbent material. U.S. Pat. Nos. 6,713,460 and 6,444,653, both to Huppe et al., describe a superabsorbent material comprising glass-like polysaccharides. Furthermore, diapers having varying degrees of biodegradability have been disclosed. U.S. Pat. No. 5,783,504 to Ehret et al. describes a composite structure, which is suitable for use in diapers, comprising a nonwoven manufactured from a polymer derived from lactic acid and a film manufactured from a biodegradable aliphatic polyester polymer. International Patent Publication No. WO 1999/33420 discloses a superabsorbent material comprising a renewable and/or biodegradable raw material. However, these diapers and materials tend to have significantly lower performance and/or higher cost than materials derived from petrochemicals. For example, the superabsorbent materials disclosed in WO 1999/33420 show a low absorption capacity under load and a low gel strength. A superabsorbent material with low gel strength tends to deform upon swelling and reduce interstitial spaces between the superabsorbent particles. This phenomenon is known as gel-blocking. Once gel-blocking occurs, further liquid uptake or distribution takes place via a very slow diffusion process. In practical terms, gel-blocking increases the susceptibility of the absorbent article to leakage.

Accordingly, it would be desirable to provide an absorbent article which comprises a polymer derived from renewable resources, where the polymer has specific performance characteristics making the polymer particularly useful in the absorbent article. Ideally, it would be desirable to provide a consumer product including a plurality of absorbent articles comprising said polymer derived from renewable resources and a communication of a related environmental message.

### SUMMARY OF THE INVENTION

The invention relates to an absorbent article having opposing longitudinal edges, the absorbent article comprising a topsheet, a backsheet joined with the topsheet, an absorbent core disposed between the topsheet and the backsheet, and a synthetic polymer derived from a first renewable resource via at least one monomeric intermediate compound. The polymer is disposed in or incorporated into one or more elements of the absorbent article. The elements are selected from a group consisting of the absorbent core, the topsheet, the backsheet, dusting layer, fastener, and a barrier leg cuff.

For example, the invention provides an absorbent core for an absorbent article. The absorbent core has a bio-based content of at least about 50% based on the total weight of the absorbent core. Additionally, the absorbent core comprises a synthetic polymer derived from a renewable resource via a first intermediate compound selected from the group consisting of crotonic acid, propiolactone, ethylene oxide, carbon monoxide, carbon dioxide, i-propanol, butanol, butyric acid, propionic acid, methyl lactate, ethyl lactate, 2-acetoxypropanoic acid, methyl 2-acetoxypropanoate, polyhydroxybutyrate, and a polyhydroxyalkanoate comprising 3-hydroxypropionate monomers. In various embodiments, the first intermediate is crotonic acid, propiolactone, or ethylene oxide, and the synthetic polymer is optionally derived from the renewable resource via acrylic acid as a second intermediate compound. In various aspects, the first intermediate is i-propanol, butanol, or propionic acid, and the synthetic polymer is optionally derived from the renewable resource via propylene as a second intermediate compound. Alternatively (or in addition), the intermediate is 2-acetoxypropanoic acid, methyl 2-acetoxypropanoate, methyl lactate, ethyl lactate, or a polyhydroxyalkanoate comprising 3-hydroxypropionate monomers.

An absorbent core for an absorbent article also is provided, the absorbent core (a) having a bio-based content of at least about 70% based on the total weight of the absorbent core and (b) comprising a synthetic polymer derived from a renewable resource via a first intermediate compound selected from the group consisting of ethylene, n-propanol, and propylene.

The invention also includes an element of an absorbent article selected from the group consisting of a topsheet, a backsheet, a dusting layer, a fastener, and a barrier leg cuff. The element has a bio-based content of at least about 50% based on the total weight of the element and comprises a synthetic polymer derived from a renewable resource via a first intermediate compound selected from the group consisting of polyhydroxybutyrate, crotonic acid, i-propanol, and butanol,

The invention also includes an element for an absorbent article selected from the group consisting of a topsheet, a backsheet, a dusting layer, a fastener, and a barrier leg cuff, the element (a) having a bio-based content of at least about 70% based on the total weight of the element and (b) comprising a synthetic polymer derived from a renewable resource via a first intermediate compound selected from the group consisting of ethylene, n-propanol and propylene.

One or more of the intermediates are derived from a suger (e.g., xylose and/or glucose) via biofermentation.

The invention also relates to a method for making an absorbent article comprising the steps of providing a renewable resource, deriving an intermediate monomeric compound from the renewable resource, polymerizing the monomeric compound to form a synthetic polymer, and disposing or incorporating the polymer into one or more elements of the absorbent article. The elements are selected from a group consisting of the absorbent core, the topsheet, the backsheet, fastener, dusting layer, and a barrier leg cuff.

In various aspects, the invention provides a method for making an element for an absorbent article. The method comprises (a) converting an intermediate to acrylic acid or propylene, wherein the intermediate is derived from biomass and is selected from the group consisting of crotonic acid, propiolactone, ethylene oxide, i-propanol, butanol, 2-acetoxypropanoic acid, methyl 2-acetoxypropanoate, methyl lactate, ethyl lactate, polyhydroxybutyrate, a polyhydroxyalkanoate comprising 3-hydroxypropionate monomers, and a blend of polyhydroxybutyrate and polyhydroxyalkanoate comprising 3-hydroxypropionate monomers; (b) polymerizing the acrylic acid or propylene to form a synthetic polymer; and (c) disposing or incorporating the synthetic polymer into the element. Several routes exist for generating acrylic acid or propylene from the intermediate. In various embodiments, the method comprises converting the propylene to acrylic acid. The propylene is optionally obtained from i-propanol to propylene via dehydration; from butene via metathesis (the butene optionally generated from butanol via dehydration); or from crotonic acid via decarboxylation (the crotonic acid optionally generated from polyhydroxybutyrate via thermolysis). When ethylene oxide is an intermediate, the method optionally comprises converting ethylene oxide to propiolactone via carbonylation, and converting the propiolactone to acrylic acid. When crotonic acid is an intermediate, the method optionally comprises converting crotonic acid to acrylic acid and/or propylene via metathesis. When the intermediate is polyhydroxyalkanoate comprising 3-hydroxypropionate monomers, polyhydroxybutyrate, or a blend of polyhydroxybutyrate and polyhydroxyalkanoate comprising 3-hydroxypropionate monomer, the method optionally comprises (i) converting the polyhydroxyalkanoate comprising 3-hydroxypropionate monomers, polyhydroxybutyrate, or blend of polyhydroxybutyrate and polyhydroxyalkanoate comprising 3-hydroxypropionate monomer to crotonic acid via thermolysis and converting the crotonic acid to acrylic acid via metathesis, or (ii) converting the polyhydroxyalkanoate comprising 3-hydroxypropionate monomers, polyhydroxybutyrate, or blend of polyhydroxybutyrate and polyhydroxyalkanoate comprising 3-hydroxypropionate monomer to acrylic acid via thermolysis. When propionic acid is an intermediate, the method optionally comprises converting propionic acid to acrylic acid via dehydrogenation. Alternatively, the method comprises (a) converting ethylene to acrylic acid via reaction with carbon dioxide; (h) polymerizing the acrylic acid to form a synthetic polymer; and (c) disposing or incorporating the synthetic polymer into the element.

### BRIEF DESCRIPTION OF THE DRAWINGS

FIG. 1A is a plan view of an exemplary absorbent article in the form of a diaper in a flat uncontracted state.

FIG. 1B is a cross-sectional view of the diaper of FIG. 1A taken along the lateral centerline.

FIGS. 2A-B are perspective views of a package comprising an absorbent article.

FIGS. 3A-F are illustrations of several suitable embodiments of icons communicating reduced petrochemical dependence and/or environmental friendliness.

FIG. 4 is a partial cross-sectional side view of a suitable permeability measurement system for conducting the Saline Flow Conductivity Test.

FIG. 5 is a cross-sectional side view of a piston/cylinder assembly for use in conducting the Saline Flow Conductivity Test.

FIG. 6 is a top view of a piston head suitable for use in the piston/cylinder assembly shown in FIG. 5.

FIG. 7 is a cross-sectional side view of the piston/cylinder assembly of FIG. 5 placed on a fritted disc for the swelling phase.

### DETAILED DESCRIPTION OF THE INVENTION

The invention relates to an absorbent article comprising a synthetic polymer derived from a renewable resource where the polymer has specific performance characteristics. When the synthetic polymer derived from a renewable resource is in the form of a superabsorbent polymer, it exhibits an Absorption Against Pressure (AAP) value of at least about 15.0 g saline per gram polymer and/or a saline flow conductivity (SFC) of at least about 30×10⁻⁷ cm³-sec/g. When the polymer is a polyolefin nonwoven suitable for use as a topsheet, it may exhibit a Liquid Strike Through value of less than about 4 seconds. When the polymer is a polyolefin nonwoven suitable for use as a barrier leg cuff, it may exhibit a hydrohead of at least about 5 mbar. When the polymer is a breathable polyolefin film suitable for use as a backsheet, it may exhibit a Moisture Vapor Transmission Rate of at least about 2000 g/m²/24 hr. When the polymer is a polyolefin film suitable for use as a backsheet, it may have an MD tensile strength of at least about 0.5 N/cm.

In another aspect, the absorbent article comprises a synthetic polymer derived from a renewable resource wherein the polymer has a ¹⁴C/C ratio of about 1.0×10¹⁴ or greater.

One or more elements of the absorbent article (e.g., the absorbent core, core wrap, topsheet, dusting layer, backsheet, barrier leg cuff, and/or fastening system) comprise sufficient levels of synthetic polymer derived from renewable bio-based resources to have a bio-based content of at least about 50%. In various aspects, the elements(s) of the absorbent article comprise at least about 60%, at least about 65%, at least about 70%, at least about 75%, at least about 80%, at least about 85%, at least about 90%, or at least about 95% bio-based content.

The absorbent article is advantageous, at least in part, because it has the same look and feel as similar articles made from virgin petroleum-based sources and similar performance characteristics as the articles made from virgin petroleum-based sources, yet the article has improved sustainability over articles derived from virgin petroleum-based sources. By “sustainable” is meant an improvement of greater than 10% in some aspect of its Life Cycle Assessment or Life Cycle Inventory, when compared to the relevant virgin petroleum-based material that would otherwise have been used to manufacture the article. As used herein, “Life Cycle Assessment” (LCA) or “Life Cycle Inventory” (LCI) refers to the investigation and evaluation of the environmental impacts of a given product or service caused or necessitated by its existence. The LCA or LCI can involve a “cradle-to-grave” analysis, which refers to the full Life Cycle Assessment or Life Cycle Inventory from manufacture (“cradle”) to use phase and disposal phase (“grave”). All inputs and outputs are considered for all the phases of the life cycle. Alternatively. LCA or LCI can involve a “cradle-to-gate” analysis, which refers to an assessment of a partial product life cycle from manufacture (“cradle”) to the factory gate (i.e., before it is transported to the customer). Alternatively, this second type of analysis is also termed “cradle-to-cradle.”

The invention further relates to a package comprising at least one absorbent a comprising a synthetic polymer derived from a renewable resource and an overwrap securing the absorbent article(s). The absorbent article comprises a synthetic polymer derived from a renewable resource. The package may further comprise a communication of a related environmental message.

The invention further relates to a method for making absorbent articles comprising a synthetic polymer derived from a renewable resource. The method comprises the following steps: providing a renewable resource; deriving at least one intermediate compound from the renewable resource, wherein the intermediate compound comprises a monomeric compound; polymerizing the monomeric compound to form at least one polymer, wherein the at least one polymer exhibits the requisite performance for use in an absorbent article; and incorporating the at least one polymer into an absorbent article.

Also included is method for making an element for an absorbent article. The method comprises converting an intermediate to acrylic acid or propylene, wherein the intermediate is derived from biomass and is selected from the group consisting of crotonic acid, propiolactone, ethylene oxide, i-propanol, butanol, methyl lactate, ethyl lactate, 2-acetoxypropanoic acid, methyl 2-acetoxypropanoate, polyhydroxybutyrate, a polyhydroxyalkanoate comprising 3-hydroxypropionate monomers, and a blend of polyhydroxybutyrate and polyhydroxyalkanoate comprising 3-hydroxypropionate monomers; polymerizing the acrylic acid or propylene to form a synthetic polymer; and disposing or incorporating the synthetic polymer into the element.

Additional steps, as described herein, may be incorporated into the method. Optionally the at least one polymer may be modified after the polymerization step.

### I. DEFINITIONS

As used herein, the following terms shall have the meaning specified thereafter:

“Disposable” refers to items that are intended to be discarded after a limited number of uses, frequently a single use (i.e., the original absorbent article as a whole is not intended to be laundered or reused as an absorbent article, although certain materials or portions of the absorbent article may be recycled, reused, or composted). For example, certain disposable absorbent articles may be temporarily restored to substantially full functionality through the use of removable/replaceable components but the article is nevertheless considered to be disposable because the entire article is intended to be discarded after a limited number of uses,

“Absorbent article” refers to devices which absorb and contain body exudates and, more specifically, refers to devices which are placed against or in proximity to the body of the wearer to absorb and contain the various exudates discharged from the body. Exemplary absorbent articles include diapers, training pants, pull-on pant-type diapers (i.e., a diaper having a pre-formed waist opening and leg openings such as illustrated in U.S. Pat. No. 6,120,487), refastenable diapers or pant-type diapers, incontinence briefs and undergarments, diaper holders and liners, feminine hygiene garments such as panty liners (e.g., such as disclosed in U.S. Pat. Nos. 4,425,130; 4,687,478; 5,267,992; and 5,733,274), absorbent inserts, and the like. Absorbent articles may be disposable or may contain portions that can be reused or restored.

“Proximal” and “Distal” refer, respectively, to the location of an element relatively near to or far from the longitudinal or lateral centerline of a structure (e.g., the proximal edge of a longitudinally extending element is located nearer to the longitudinal centerline than the distal edge of the same element is located relative to the same longitudinal centerline).

“Body-facing” and “garment-facing” refer respectively to the relative location of an element or a surface of an element or group of elements. “Body-facing” implies the element or surface is nearer to the wearer during wear than some other element or surface. “Garment-facing” implies the element or surface is more remote from the wearer during wear than some other element or surface (i.e., element or surface is proximate to the wearer's garments that may be worn over the absorbent article).

“Superabsorbent” refers to a material capable of absorbing at least ten times its dry weight of a 0.9% saline solution at 25° C. Superabsorbent polymers absorb fluid via an osmotic mechanism to form a gel, often referred to as, and used interchangeably with the term “hydrogel.”

“Longitudinal” refers to a direction running substantially perpendicular from a waist edge to an opposing waist edge of the article and generally parallel to the maximum linear dimension of the article. Directions within 45 degrees of the longitudinal direction are considered to be “longitudinal.”

“Lateral” refers to a direction running from a longitudinal edge to an opposing longitudinal edge of the article and generally at a right angle to the longitudinal direction. Directions within 45 degrees of the lateral direction are considered to be “lateral.”

“Disposed” refers to an element being located in a particular place or position.

“Joined” refers to configurations whereby an element is directly secured to another element by affixing the element directly to the other element and to configurations whereby an element is indirectly secured to another element by affixing the element to intermediate members) which in turn are affixed to the other element.

“Film” refers to a sheet-like material wherein the length and width of the material far exceed the thickness of the material. Typically, films have a thickness of about 0.5 mm or less.

“Impermeable” generally refers to articles and/or elements that are not penetrative by fluid through the entire Z-directional thickness of the article under pressure of 0.14 lb/in² or less. Preferably, the impermeable article or element is not penetrative by fluid under pressures of 0.5 lb/in² or less. More preferably, the impermeable article or element is not penetrative by fluid under pressures of 1.0 lb/in² or less. The test method for determining impermeability conforms to Edana 120.1-18 or INDA IST 80.6.

“Extendibility” and “extensible” mean that the width or length of the component in a relaxed state can be extended or increased by at least about 1096 without breaking or rupturing when subjected to a tensile force.

“Elastic,” “elastomer,” and “elastomeric” refer to a material which generally is able to extend to a strain of at least 50% without breaking or rupturing. and is able to recover substantially to its original dimensions after the deforming force has been removed.

“Elastomeric material” is a material exhibiting elastic properties. Elastomeric materials may include elastomeric films, scrims, nonwovens, and other sheet-like structures.

“Outboard” and “inboard” refer respectively to the location of an element disposed relatively far from or near to the longitudinal centerline of the diaper with respect to a second element. For example, if element A is outboard of element B, then element A is farther from the longitudinal centerline than is element B.

“Pant” refers to an absorbent article having a pre-formed waist and leg openings. A pant may be donned by inserting a wearer's legs into the leg openings and sliding the pant into position about the wearer's lower torso. Pants are also commonly referred to as “closed diapers,” “prefastened diapers,” “pull-on diapers,” “training pants” and “diaper-pants.”

“Petrochemical” refers to an organic compound derived from petroleum, natural gas, or coal.

“Petroleum” refers to crude oil and its components of paraffinic, cycloparaffinic, and aromatic hydrocarbons. Crude oil may be obtained from tar sands, bitumen fields, and oil shale.

“Renewable resource” refers to a natural resource that can be replenished within a 100 year time frame. The resource may be replenished naturally, or via agricultural techniques. Renewable resources include plants, animals, fish, bacteria, fungi, and forestry products. They may be naturally occurring, hybrids, or genetically engineered organisms. Natural resources such as crude oil, coal, and peat which take longer than 100 years to replenish or form are not considered to he renewable resources

“Bio-based content” refers to the amount of bio-carbon in a material as a percent of the weight (mass) of the total organic carbon in the product. For example, polyethylene contains two carbon atoms in its structural unit. If ethylene is derived from a renewable resource, then a homopolymer of polyethylene theoretically has a bio-based content of 100% because all of the carbon atoms are derived from a renewable resource. A copolymer of polyethylene could also theoretically have a bio-based content of 100% if both the ethylene and the co-monomer arc each derived from a renewable resource. In embodiments where the co-monomer is not derived from a renewable resource, the polymer will typically include only about 1 wt. % to about 2 wt. % of the non-renewable co-monomer, resulting in polymer having a theoretical bio-based content that is slightly less than 100%. As another example, polyethylene terephthalate contains ten carbon atoms in its structural unit (i.e., two from the ethylene glycol monomer and eight from the terephthalic acid monomer). If the ethylene glycol portion is derived from a renewable resource, but the terephthalic acid is derived from a petroleum-based resource, the theoretical bio-based content of the polyethylene terephthalate is 20%.

“Agricultural product” refers to a renewable resource resulting from the cultivation of land (e.g., a crop) or the husbandry of animals (including fish).

“Monomeric compound” refers to an intermediate compound that may be polymerized to yield a polymer.

“Polymer” refers to a macromolecule comprising repeat units where the macromolecule has a molecular weight of at least 1000 Daltons. The polymer may be a homopolymer, copolymer, terpolymer etc. The polymer may be produced via free-radical, condensation, anionic, cationic. Ziegler-Natta, metallocene, or ring-opening mechanisms. The polymer may be linear, branched and/or crosslinked.

“Synthetic polymer” refers to a polymer which is produced from at least one monomer by a chemical process. A synthetic polymer is not produced directly by a living organism.

“Sugar” refers to five carbon monosaccharides (C5 or pentose; e.g., xylose, arabinose, or ribose), six carbon monosaccharides (C6 or hexose; e.g., glucose, fructose, or mannose), disaccharides (e.g., sucrose), or blends of any of the foregoing.

“Polyethylene” and “polypropylene” refer to polymers prepared from ethylene and propylene, respectively. The polymer may be a homopolymer, or may contain up to about 10 mol % of repeat units from a co-monomer,

“Communication” refers to a medium or means by which information, teachings, or messages are transmitted.

“Related environmental message” refers to a message that conveys the benefits or advantages of the absorbent article comprising a polymer derived from a renewable resource. Such benefits include being more environmentally friendly, having reduced petroleum dependence, being derived from renewable resources, and the like.

All percentages herein are by weight unless specified otherwise.

### II. POLYMERS DERIVED FROM RENEWABLE RESOURCES

A number of renewable resources contain polymers that are suitable for use in an absorbent article (i.e., the polymer is obtained from the renewable resource without intermediates). Suitable extraction and/or purification steps may be necessary, but no intermediate compound is required. Such polymers derived directly from renewable resources include cellulose (e.g., pulp fibers), starch, chitin, polypeptides, poly(lactic acid), polyhydroxyalkanoates, and the like. These polymers may be subsequently chemically modified to improve end use characteristics (e.g., conversion of cellulose to yield carboxycellulose or conversion of chitin to yield chitosan). However, in such cases, the resulting polymer is a structural analog of the starting polymer. Polymers derived directly from renewable resources (i.e., with no intermediate compounds) and their derivatives are known and these materials are not within the scope of the invention.

The synthetic polymers of the invention are derived from a renewable resource via an indirect route involving one or more intermediate compounds. Suitable intermediate compounds derived from renewable resources include sugars. Sugars may be readily produced from renewable resources, such as sugar cane and sugar beets. Sugars may also be derived (e.g., via enzymatic cleavage or other methods) from other agricultural products, such as starch or cellulose. For example, glucose may be prepared on a commercial scale by enzymatic hydrolysis of corn starch. While corn is a renewable resource in North America, other common agricultural crops may be used as the base starch for conversion into glucose. Wheat, buckwheat, arracaha, potato, barley, kudzu, cassava, sorghum, sweet potato, yam, arrowroot, sago, and other like starchy fruit, seeds, or tubers are may also be used in the preparation of glucose.

Other suitable intermediate compounds derived from renewable resources include monofunctional alcohols, such as methanol or ethanol, and polyfunctional alcohols, such as glycerol. Ethanol may be derived from many of the same renewable resources as glucose. For example, cornstarch may be enzymatically hydrolyzed to yield glucose and/or other sugars. The resultant sugars can be converted into ethanol by fermentation. As with glucose production, corn is an ideal renewable resource in North America; however, other crops may be substituted. Methanol may be produced from fermentation of biomass. Glycerol is commonly derived via hydrolysis of triglycerides present in natural fats or oils, which may be obtained from renewable resources, such as animals or plants.

Other intermediate compounds derived from renewable resources include organic acids (e.g., citric acid, lactic acid, alginic acid, amino acids etc.), aldehydes (e.g., acetaldehyde), and esters (e.g., cetyl palmitate, methyl stearate, methyl oleate, etc.).

Additional intermediate compounds such as methane and carbon monoxide may also be derived from renewable resources by fermentation and/or oxidation processes. Other exemplary intermediate compounds described herein include, e.g., crotonic acid, propiolactone, ethylene, ethylene oxide, n-propanol, i-propanol butanol, butyric acid, propionic acid, propylene, 2-acetoxypropanoic acid, methyl 2-acetoxypropanoate, methyl lactate, ethyl lactate, polyhydroxybutyrate, and a polyhydroxyalkanoate comprising 3-hydroxypropionate monomers. Particularly desirable intermediates include (meth)acrylic acids and their esters and salts; and olefins.

Intermediate compounds derived from renewable resources may be converted into polymers (e.g., glycerol to polyglycerol) or they may be converted into other intermediate compounds in a reaction pathway which ultimately leads to a polymer useful in an absorbent article. An intermediate compound may be capable of producing more than one secondary intermediate compound. Similarly, a specific intermediate compound may be derived from a number of different precursors, depending upon the reaction pathways utilized. As used herein with respect to intermediates, “first” and “second” serve to distinguish different intermediates in a reaction pathway, and do not necessarily denote the sequences in which intermediates are formed.

In various embodiments, an intermediate compound in the production of the synthetic polymer is acrylic acid, ethylene, or propylene. Acrylic acid, ethylene, or propylene may be derived from renewable resources via a number of suitable routes. Examples of such routes are provided below. Various routes are described below separately merely for convenience; it will be appreciated that one or more features of one or more routes can be combined or substituted to generate a desired intermediate, increase yield, or produce the synthetic polymer.

### A. Acrylic Acid

Materials and method for producing acrylic acid, including methods utilizing renewable resources as starting material, are described below.

1. Glycerol

Glycerol derived from a renewable resource (e,,, via hydrolysis of soybean oil and other triglyceride oils; via fermentation (see, e.g., Wang et al., Biotechnology Advances, 19, 201-223 (2001)); or via hydrogenation of C5 sugars, hydrogenolysis of the resultant polyols, and removal of glycerin from the product mix that includes ethylene glycol and propylene glycol) is converted into acrylic acid using any of a number of conversion methodologies. In one aspect, glycerol is converted to acrylic acid using a two-step process. In a first step, the glycerol is converted to an acrolein intermediate, which is converted to acrylic acid.

In various embodiments, glycerol is converted to acrolein via dehydration. A particularly suitable conversion process involves subjecting glycerol in a gaseous state to an acidic solid catalyst such as H₃PO₄ on an aluminum oxide carrier (which is often referred to as solid phosphoric acid) to yield acrolein. Specifics relating to dehydration of glycerol to yield acrolein are disclosed, for instance, in U.S. Pat. Nos. 2,042,224 and 5,387,720.

Other process conditions and catalysts for dehydrating glycerol to produce acrolein have been described in the art. For example, European Patent No. 2006273 discloses a process for preparing acrolein from glycerol which involves dehydrating glycerin gas in the presence of a catalyst containing a metal phosphate (e.g., aluminum phosphate, zirconium phosphate, manganese phosphate, or alkali metal phosphates). International Patent Publication No. 2010/046227 describes a catalyst system comprising vanadium phosphate, boron phosphate or aluminum phosphate. The dehydration reaction is, in various instances, carried out in the gas phase in the presence of oxygen starting from aqueous solutions of glycerol. Use of a catalyst comprising a boron salt of phosphoric acid and/or a zinc salt of phosphoric acid is described in, e.g., Japanese Patent Publication No. 2009263284. Use of a crystalline metallosilicate catalyst to dehydrate glycerol to acrolein is disclosed in, e.g., International Patent Publication No. WO 2007132926. Japanese Patent Publication No. 2008137950 describes solid acid catalysts for glycerol dehydration. The solid acid catalyst is a crystalline metallosilicate, a metallic oxide or argillite. The metal of the catalyst is chosen from Pt, Pd, Ru, Rh, Ir, and Cu. Still more suitable catalysts and process conditions are disclosed in Japanese Patent Publication No. 2009274982, which describes the dehydration of gaseous glycerol to acrolein catalyzed by a rare earth metal (e.g., Y, La, Ce, Pr, or Nd) salt (e.g., nitrate, carbonate, chloride, or organic acid) crystal of phosphoric acid. Tantalic acid-based catalysts are described in e.g., Japanese Patent Publication No. 2010155183. Still more dehydration catalysts (e.g., Al, B, Ti, Cr, Fe, Ni, Cu, Zn, Ga, In, P, Sc, V, Ge, crystalline metallosilicate, sulfuric acid metal salt, an oxygen-containing aluminum compound, a zirconium compound, a titanium compound, a silicon compound, a sulfur compound, a phosphorus compound, a tungsten compound, a heteropoly acid of silicon, a heteropoly acid of molybdenum, a heteropoly acid of tungsten, or a heteropoly acid of phosphorus) and dehydration conditions (gaseous glycerol) are described in Japanese Patent Publication No. 2007268364 and German Patent Publication No. 102008038273.

Additional catalysts are described in, e.g., U.S. Patent Application Publication No. 2011/0082319, which describes solid acid catalysts having a Hammett acidity of less than +2. Exemplary catalysts include zeolites, Nafion® composites, chlorinated aluminas, phosphotungstic and/or silicotungstic acids and acid salts, metal oxides, such as tantalum oxide, niobium oxide, alumina, titanium oxide, zirconia, tin oxide, silica or silicoaluminate, impregnated with acid functional groups, such as borate, sulfate, tungstate, phosphate, silicate or molybdate. The dehydration reaction is optionally carried out in the gas phase at a temperature of 150° C.-500° C. and at a pressure of between 1 and 5 bar in the presence of SO₃, SO₂, or NO₂.

Japanese Patent Publication No. 2008273885 describes an alternative catalyst for dehydration of glycerol to acrolein. The catalyst comprises phosphorus (P) and at least one alkali metal (M) selected from sodium, potassium, and cesium, wherein the M/P molar ratio is no higher than 2.0. The catalyst is prepared by impregnating a support with an aqueous solution containing phosphoric acid and at least one alkali metal ion selected from Na⁺, K⁺, and Cs⁺, and drying and solidifying the impregnated support.

In various embodiments, the catalyst is pretreated prior to exposure to glycerin to increase acrolein yield or enhance stability. Japanese Patent Publication No. 2008137952 describes an exemplary pre-treatment method, wherein a solid phase catalyst is contacted with an organic compound other than glycerin before exposure to gaseous phase glycerin. The conditions under which glycerin is exposed to catalyst for dehydration will depend on the catalyst, the raw material, the presence of byproducts, and the like. In one aspect, glycerol is reacted with a solid acid catalyst in the presence of a volatile organic acid (e.g., a carboxylic acid with three or less carbons), resulting in the production of acrolein via gaseous phase dehydration, as described in published Japanese Application No. 2010095484. U.S. Patent Publication No. 20100105957 discloses a method of producing acrolein from glycerol gas by contacting glycerin gas with a solid acid catalyst (e.g., crystalline aluminum phosphate) in a reactor. Lowering the partial pressure of glycerin gas in the raw material gas in the gas-phase dehydration of glycerin improves acrolein yield and extends the half life of the catalyst. In this regard, the partial pressure of the glycerin gas in the raw material gas is from 0.01 to 30 kPa, the space velocity of the glycerin is preferably from 70 to 2,400 hr⁻¹, the feed amount of glycerin gas per 1 L of the catalyst is preferably from 300 to 15,000 g/hr, and the reaction temperature is preferably about 350° C. to about 460° C.

The glycerol starting material may be treated or processed prior to exposure to a catalyst and/or the dehydration reaction can be repeated to ensure maximal conversion to acrolein. For example, in one aspect, a glycerin composition (e.g., unrefined glycerin obtained from naturally-occurring resources such as fatty oil was(e) is separated into a glycerin-containing gas and a non-gas component having a boiling point higher than that of glycerin, as described further in Japanese Patent Publication No. 2008174544. The lycerin-containing gas is brought into contact with a solid acid catalyst to produce acrolein. Japanese Patent Publication No. 2008266165 describes a method for preparing acrolein from glycerin wherein gaseous glycerin is brought into contact with a dehydration catalyst (e.g., Al, B, Ti, Cr, Fe, Ni, Cu, Zn, Ga, In, P, Sc, V, Ge, crystalline metallosilicate, sulfuric acid metal salt, etc.), acrolein is collected from acrolein-containing gas, and the uncollected gas is brought into contact with the catalyst.

The raw material fed into the dehydration reaction is optionally optimized to improve half-life of the catalyst (e.g., a molybdenum- or vanadium-containing catalyst). In one aspect, glycerin-containing gas comprising a molar ratio of oxygen to glycerin in a range of not lower than 0.8 and not higher than 20 is utilized, as described in U.S. Patent Publication No. 20100010260. Exemplary conditions for gas-phase reactions include, but are not limited to, a reaction temperature of about 200° C. to about 400° C., a reaction pressure of from about 0.001 MPa to about 1 MPa, in a fixed bed reactor, moving bed reactor, or fluidized bed reactor format.

In the second step of the exemplary two-step process for producing acrylic acid from glycerol, the acrolein intermediate is converted to acrylic acid via, e.g., oxidation. A particularly suitable process involves a gas phase interaction of acrolein and oxygen in the presence of a metal oxide catalyst. A molybdenum and vanadium oxide catalyst may be used. Specifics relating to oxidation of acrolein to yield acrylic acid are disclosed, for instance, in U.S. Pat. No. 4,092,354. Japanese Patent Publication No. 2007268364 and German Patent Publication No. 102008038273 describe additional oxidation catalysts suitable for use in the context of the invention to produce acrylic acid (e.g., iron oxide, molybdenum oxide, titanium oxide, vanadium oxide, tungstic oxide, antimony oxide, tin oxide, copper oxide, molybdenum, tungsten, vanadium, antimony, copper, or a combination of any of the foregoing),

The oxidation reaction is optionally conducted in a fixed bed reactor which is loaded with catalysts, such as the format described in U.S. Patent Publication No. 2010249454. An alternative two-step method is performed in a tandem-type reactor and comprises (1) vaporizing a raw material comprising an aqueous glycerol solution having a water content of not more than 50% to generate a first gas, (2) dehydrating gaseous glycerol in the first gas, and (3) oxidizinu a gaseous reaction product formed by the dehydration reaction to obtain acrylic acid. Use of a tandem-type reactor is further described in U.S. Patent Publication No. 2010/0063233.

It will be appreciated that the process for generating acrylic acid from glycerol is not dependent on the source of the glycerol. Preferably, although not required, glycerol is derived from a renewable resource. For example, in various aspects, triglyceride (such as triglyceride obtained from vegetable oils) is combined with an alcohol of the formula R′—OH (e.g., methanol or ethanol) in the presence of a catalyst to generate a fatty acid alkyl ester and glycerol. The glycerol is then dehydrated to acrolein by a catalyst, the acrolein is isolated, and, optionally, converted to acrylic acid. The method of generating glycerol from triglycerides is further described in U.S. Patent Publication No. 2008/0119663.

Glycerol optionally is converted to acrylic acid in a single step (i.e., without an acrolein intermediate). In an exemplary process, acrylic acid is produced in a single step by an oxydehydration reaction of glycerol in the presence of molecular oxygen and a catalyst. A single catalyst capable of catalyzing both a dehydration reaction and an oxidation reaction is used in various aspects. Alternatively, a catalyst mixture is used comprising a first catalyst capable of effecting a dehydration reaction and second catalyst capable of effecting an oxidation reaction. The presence of oxygen minimizes the formation of coke on the catalyst and the formation of unwanted byproducts. The process is further described in U.S. Patent Publication No 20080183013.

An alternative exemplary method of directly converting glycerol to acrylic acid comprises subjecting aqueous glycerol to reactive vaporization in a fluidized bed containing a reactive solid at a temperature of, e.g., about 180° C. to about 400° C. The reactive solid comprises a catalyst for an oxydehydration reaction of glycerol to acrylic acid (although the reactive solid may alternatively or additionally comprise a dehydration catalyst that promotes formation of acrolein from glycerol). Thus, the glycerol is simultaneously vaporized and subjected to oxydehydration in a format that allows removal of impurities. The process is described further in U.S. Patent Publication No, 2011028760.

European Pat. No. 1710227 describes another representative method for gene ating acrylic acid wherein an aqueous glycerol solution a glycerol solution comprising a water content of 50% or less, and preferably no more than 10% water content) is vaporized and passed through a flow reactor in the presence of a catalyst (eg., natural and synthetic clay compounds such as kaolinite, bentonite montmorillonite and zeolite; phosphoric acid or sulfuric acid supported on a support; inorganic oxides or inorganic composite oxides, such as, e.g., Al₂O₃, TiO₂, ZrO₂, SnO₂, V₂O₅, SiO₂—Al₂O₃, SiO₂—TiO₂ and TiO₂—WO₃; solid acidic substance, such as, e.g., sulfates, carbonates, nitrates and phosphates of metals such as MgSO₄, Al₂(SO₄)₃, K₂SO₄, AlPO₄, and Zr₃(PO₄)₂) to dehydrate the glycerol to produce acrolein. The shape of the catalyst can be modified as desired; spheres, pillars, and rings are suitable, as are a myriad of other shapes. Next, the gaseous reaction product from the dehydration reaction is subjected to a gas phase oxidation reaction in the presence of a catalyst (e.g., kaolinite, bentonite, montmorillonite, zeolite, iron oxide, molybdenum oxide, titanium oxide, vanadium oxide, tungsten oxide, antimony oxide, tin oxide, or copper oxide, optionally supported on supports). The reactions can be performed using a tandem-type reactor wherein catalysts for the dehydration and oxidation reactions are kept separately; a single type reactor comprising one reaction tube with dehydration catalyst(s) positioned near the gas intake outlet and oxidation catalyst(s) positioned near the reaction product outlet; or a single-type reactor comprising one reaction tube wherein the multiple catalysts are intermixed.

2. Lactic Acid

Acrylic acid is derived from lactic acid (which can itself be derived from renewable resources) by several processes. For example, dehydration of lactic acid produces acrylic acid. Optionally, the dehydration reaction is facilitated by an acidic dehydration catalyst, such as an inert metal oxide carrier which has been impregnated with a phosphate salt, as further described in U.S. Pat. No. 4,729,978. Alternatively, lactic acid is converted to acrylic acid by reaction with a catalyst comprising solid aluminum phosphate, as described in further detail in U.S. Pat. No. 4,786,756. Another suitable dehydration process is described in U.S. Pat. No. 2,859,240 and involves using a sulfate or phosphate catalyst of group 1 or 2 metals. Representative sulfates and phosphates include, but are not limited to, LiH₂PO₄, CsH₂PO₄, KH₂PO₄, CaHPO₄, (NH₄)₂HPO₄, MgSO₄, NaH₂PO₄, and CaSO₄.

In another exemplary dehydration reaction, a —NaY (zeolite of faujasite structure) molecular sieve catalyst is employed. Acrylic acid is formed by adding the catalyst into a constant-temperature section of a fixed bed reactor, raising the temperature of the catalyzing bed to about 300° C. to about 450° C. under N, gas, adding lactic acid into the reactor, gasifying the lactic acid in the presence of the catalyst, and cooling and separating the acrylic acid. This process is further described in Chinese Patent Publication No, 101279910. Optionally, a fixed bed reactor is employed with a catalyst comprising 0.1-93% silicon dioxide and aluminum dioxide (i.e., HZSM-5), 6-99.8% —NaY (zeolite of fainasite s(ructure), and 0.001-1.0% potassium, and lactic acid solution is applied to the fixed reactor with inert gases at a reaction temperature of about 280° C. to about 450° C. (see Chinese Patent Publication No. 101049571). The acrylic acid is collected from the liquid phase of the product.

Direct dehydration of lactic acid to acrylic acid using a vertical fixed bed reactor is contemplated, and a representative example of the process is described in Chinese Patent Publication No. CN101306990. A vertical fixed bed reactor is prepared wherein an upper reactor comprising catalyst A (e.g., X-type molecular sieve) and catalyst B (e.g., -NaY molecular sieve) is connected to a raw material carburetor. A lower reactor comprising catalyst C (e.g., a mixture of praseodymium nitrate, neodymium nitrate, lanthanum nitrate, yttrium nitrate, and bluestone) and catalyst D (e.g., a mixture of calcium sulfate and copper sulfate) is connected with a gas-liquid separator. The reaction temperature of the upper section is about 275° C. to about 350° C. and the lower section is about 350° C. to about 450° C. Raw material (e.g., lactic acid) is pumped into the carburetor with para-hydroxybenzoic ether (to inhibit polymerization) and converted to acrylic acid, which is isolated using the gas-liquid separator.

It will be appreciated that the catalyst can be shaped for use in a selected reactor and/or to maximize yield and/or to reduce degradation or unwanted impurity build-up. An extruded catalyst, for example, can be structurally strong, exhibit high activity and good selectivity, be easy to operate, and inexpensive. A suitable extruded catalyst comprises, for example. Y molecular sieve raw powder, an adhesive, an extrusion aid, and water. The weight ratio of the Y molecular sieve raw powder to the adhesive is 2-50:1, the weight ratio of the Y molecular sieve raw powder to the extrusion aid is 10-100:1, the weight ratio of the water to the Y molecular sieve raw powder is 0.3-1:1, and various raw materials are mixed evenly. The components are kneaded, aged, extruded, and dried to obtain the extruded catalyst. Extruded catalysts are further described in e.g., Chinese Patent Publication No. 101462069.

In various embodiments, lactic acid is converted to acrylic acid via a 2-acetoxy propionic acid (APA) intermediate. In this regard, lactic acid is subjected to, e.g., reactive distillation to form APA, which is converted to acrylic acid by, e,g., pyrolysis. The invention includes conversion of lactic acid (or a lactic acid ester) to acrylic acid in a two step process wherein the lactic acid or ester is introduced into a first vessel with an excess of acetic acid. The lactic acid or lactic acid ester is reacted with a first portion of the acetic acid in the presence of a first catalyst (e.g., a strong acid catalyst, such as sulfuric acid, polysulfonic acid, polyphosphoric acid, or a solid acid catalyst) to produce a 2-acetoxy propionic acid or ester. If desired, any non-reacted portion of the acetic acid is recycled and re-used for further reaction without converting to an anhydride. The 2-acetoxy propionic acid or ester is transferred to a second vessel where, in the presence of a second catalyst (e.g., a weak acid catalyst including, but not limited to, sulfate salts and phosphate salts, such as calcium sulfate), acetic acid is liberated from the 2-acetoxy propionic acid or ester. Acrylic acid or a first acrylate ester is produced. The reactions are optionally performed a single reaction vessel or, alternatively, are performed in separate vessels. In this regard, the first reaction step comprises esterifying lactic acid to produce the alpha-acyloxy derivative (i.e., APA) in a first reaction vessel, and the APA is transferred to a second reaction vessel for conversion to acrylic acid. Water formed during the process is optionally removed by, for example, providing one or more substances that form an azeotrope with water or chemically react with water. Materials and methods for converting lactic acid to acrylic acid via APA are further described in U.S. Pat. No. 6,992,209.

German Patent Application No. 2046411 describes a process of making acrylic acid by passing 2-acetoxy-propionic acid (APA) over catalysts that contain group 1 and/or 2 phosphates (PO₄) at a temperature of about 200° C. to about 500° C.

Alternatively or in addition, lactic acid is converted to acrylic acid through a multiple step reaction scheme. In one variation, lactic acid is converted to ethyl-2-hydroxypropionate, which is dehydrated to form ethyl acrylate, which is hydrolyzed to form acrylic acid. See, e.g., Burns et al., J. Chem. Soc., 400-406 (1935). Another variation employs a methyl 2-acetyloxypropanoate (MAPA) intermediate. The chemical conversions of the process can be achieved by any suitable means. For example, German Patent Publication No. 4340369 describes a process for preparing an acylated α-hydroxyalkanoate ester comprising reacting an α-hydroxy ester with an alkanoic acid in the gas phase with a solid acid as a heterogeneous catalyst. The reaction product is pyrolized to form an acrylic ester. One suitable (but not limiting) method of forming an acrylate ester from a derivative of α-acetoxypropionic acid is described in Burns et al., J. Chem. Soc., 400-406 (1935), which details generating methyl acrylate via pyrolysis of methyl α-acetoxypropionate at temperatures of about 500° C. Methods of producing acrylic acid from methyl acrylate also are known in the art and include, for instance, acidolysis in the presence of formic or acetic acid, sulfuric acid, and hydroquinone, as described in Ratchford et al., J. Am. Chem. Soc., 66. 1864-1866 (1944). Exemplary conditions for the reaction are as follows: four moles of formic acid, 12 moles of methyl acrylate, 30 g of hydroquinone and 2 cc. of sulfuric acid is mixed and refluxed in a still having a 3 foot column and variable take-off head. The still is operated with total reflux until the temperature of the head falls to 32° C. Methyl formate is withdrawn for 8-12 hours (until its production ceases). Excess methyl acrylate is distilled at a pressure of 140 mm and acrylic acid is distilled at 56° C. The process conditions described are merely illustrative; one of ordinary skill has the requisite knowledge to adapt the process conditions for a particular apparatus or to achieve a desired yield.

Optionally, lactic acid is esterified to form, e.g., methyl lactate or ethyl lactate, which is converted to acrylic acid or methyl acrylic acid. In various embodiments, acrylate is generated from methyl lactate by exposing the lactate ester to a catalyst, such as crystalline hydrated and partially calcined calcium sulfate as described in, for example, U.S. Pat. No. 5,071,754, at high temperatures. When utilizing a solid bed of crystalline hydrated and partially calcined calcium sulfate, the lactic acid ester is preferably exposed to the catalyst at temperature of from about 350° C. to about 410° C. at substantially atmospheric pressure. Calcium phosphate and calcium pyrophosphate composite catalysts also are suitable for converting methyl lactate to acrylic acid under vapor phase reaction conditions. Calcium phosphate and calcium pyrophosphate composite catalysts and conditions for using the catalysts to convert methyl lactate to acrylic acid are further described in, e.g., Hong et al., Applied Catalysts A: General, 396, 194-200 (2011).

The methods of producing acrylic acid from lactic acid are not dependent on the source of the lactic acid starting material, although the lactic acid starting material is desirably derived from renewable resources. In various aspects, lactic acid is derived from monosaccharides (e.g., glucose), which optionally is derived from biomass or other plant-based carbohydrate material, such as black-strap molasses, sugar cane juice, sugar beet juice, potato processing wastes, whey, hydrolyzed wood, grass, switch grass, cord grass, rye grass, reed canary grass, mixed prairie grass, miscanthus, sugar-processing residues, sugar cane bagasse, sugarcane straw, agricultural wastes, rice straw, rice hulls, barley straw, corn cobs, cereal straw, wheat straw, canola straw, oat straw, oat hulls, corn fiber, stover, soybean stover, corn stover, forestry wastes, recycled wood pulp fiber, paper sludge, sawdust, hardwood, softwood, and combinations thereof. Depending on the starting material, the biomass or other carbohydrate material may be pre-treated with steam and/or acid to obtain a degraded carbohydrate material, which is further degraded enzymatically using, e,g, amylase, glucosidase, and/or cellulase. The resulting monosaccharide is e.g., biofermented to yield lactic acid, which is converted to acrylic acid using any suitable method, such as the methods described herein. Any suitable microorganism capable of fermenting glucose to yield lactic acid is appropriate for use in the context of the invention, including acidophilus bacteria and members from the genus Lactobacillus such as Lactobacillus lactis, Lactobacillus delbruckii and Lactobacillus buloricus. Biofermentation of sugars to yield lactic acid is further described in, e.g., U.S. Pat. Nos. 5,464,760 and 5,252,473,

### 3. 3-Hydroxypropionic Acid

3-Hydroxypropionic acid (3-HP) is another suitable intermediate for generating acrylic acid. Generally, acrylic acid is produced from 3-HP via dehydration, which can be achieved under a number of reaction conditions. One exemplary method of generating acrylic acid comprises providing 3-HP (or other β-hydroxy carbonyl compound) under generally continuous flow to a reactor packed with a dehydration catalyst in a fixed-bed configuration. The 3-HP is heated in the presence of the catalyst to e.g., about 100° C. to about 250° C., for a time sufficient to dehydrate the material in contact with the catalyst, yielding acrylic acid at high conversion and molar yield. The flow rate and temperature are selected to enable the material to substantially react with the catalyst inside the reactor in the absence of an inert gas flow. Suitable catalysts include, but are not limited to, solid oxides, solid acids, acidic catalysts, weakly acidic catalysts, strongly acidic catalysts, basic catalysts, ion-exchange resins, acidic gases, basic gases, or combinations thereof. The solid oxide catalyst is optionally TiO₂, ZrO₂, Al₂O₃, SiO₂, ZnO₂, SnO₂, WO₃, MnO₂, Fe₂O₃, SiO₂/Al₂O₃, ZrO₂/WO₃, ZrO₂/Fe₂O₃, ZrO₂/MnO₂, or combinations thereof. Acidic or weakly acidic catalysts are optionally selected from titanic acids, metal oxide hydrates, metal sulfates, metal oxide sulfates, metal phosphates, metal oxide phosphates, mineral acids, carboxylic acids, salts of any of the foregoing, acidic resins, acidic zeolites, clays, carbon dioxide, or combinations thereof (such as Ti-0720™, SiO₂/H₃PO₄, fluorinated Al₂O₃, Nb₂O₃/PO₄⁻³, Nb₂O₃/SO₄⁻², Nb₂O₅H₂O, phosphotungstic acids, phosphomolybdic acids, silicomolybdic acids, silicotungstic acids, carbon dioxide, salts thereof, PVPH⁺Cl^{-™}, or combinations thereof). Exemplary basic catalysts include, but are not limited to, ammonia, polyvinylmidine, metal hydroxides. Zr(OH)₄, or amines of the form NR₁R₂R₃, wherein R₁. R₂, and R₃ are selected from the group consisting of H, alkyl and aryl groups containing from 1 to 20 carbon atoms, or combinations thereof. The process is described in further detail in U.S. Patent Application Publication No. 20070219390.

In a related process, a salt of 3-HP is used to generate an acrylate salt via dehydration, optionally in a non-aqueous state, in the presence of a catalyst. In one embodiment, the starting material is melt of a 3-HP salt and the reaction is performed in the presence of a polymerization inhibitor, such as a phenolic compound (e.g., dimethoxyphenol (DMP) or alkylated phenolic compound such as di-tert-butyl phenol), quinone (e.g., t-butyl hydroquinone or the monomethyl ether of hydroquinone (MEHQ)), and/or metallic copper or copper salt copper sulfate, copper chloride, or copper acetate). The 3-HP salt is optionally an alkali salt, alkaline earth salt, transition metal salt, precious metal salt, or ammonium salt. The catalyst is optionally a solid oxide catalyst, solid acid catalyst, acidic catalyst, weakly acidic catalyst, strongly acidic catalyst, basic catalyst, ion-exchange resin, acidic gas, or basic gas. This process is described in detail in U.S. Pat. No. 7,687,661. Similar materials and methods are employed to convert an ammonium salt 3-HP (or any β-hydroxy carbonyl compound) to acrylic acid using a dehydration catalyst, as further described in U.S. Patent Application Publication No. 20070219397. An additional process for generating acrylic acid from 3-HP via dehydration is provided in International Patent Publication No. WO 2010090322, which describes methods of deriving hydrophilic polyacrylic acid salt resins. The process comprises, in one aspect, 3-HP dehydration using phosphoric acid.

In another exemplary process, 3-HP is converted to acrylamide by heating an aqueous solution of 3-HP in the presence of a 3-HP amide and an amidation and/or dehydration catalyst. The dehydration step for formation of the unsaturated acid can be carried out in the vapor phase or the liquid phase at a temperature from about 100° C. to about 400° C. Additionally the reaction can be carried out in supercritical reaction media. Vapor phase reactions normally require higher temperatures than liquid phase reactions. For example, a solution of 20% 3-hydroxypropionic acid (3-HP) in water containing 100 ppm of p-methoxyphenol is fed at a rate of 15 grams per hour into the top of a vertical silica reactor tube containing a 500 mm bed of ceramic packing. The bottom 25 mm of the bed consists of a high surface area gamma-alumina packing, and there is a simultaneous flow of 60 ml/min of nitrogen. The tube is heated by a concentric tube furnace to 250° C. The gaseous effluent from the bottom of the tube is condensed and collected. This process is described in further detail in U.S. Pat. No. 7,538,247.

Acrylic acid can be recovered from unreacted 3-HP using any suitable method, such as the method described in International Patent Publication No. WO 2005021470 using an organic extractant. The aqueous 3-HP solution remaining after acrylic acid extraction by the organic extractant is subjected to further processing to increase the yield of acrylic acid, if desired. Organic extractants include. e.g., an alcohol, an ether, an ester, a ketone, an amide, a phosphorus ester, a halogenated compound, an aromatic compound, a phosphine oxide, a phosphine sulfide, an alkyl sulfide, and mixtures thereof. In various aspects, the organic extrant is not ethyl acetate. Acrylic acid is purified from the solution containing the extractant by back extraction with water, or distilling the solution in the presence of water, thereby resulting in an aqueous acrylic acid solution.

3-HP can be obtained from any source, including renewable sources such as carbohydrate material derived from, e.g., plant material. An environmentally-friendly method of obtaining 3-HP comprises biofermenting sugar, such as glucose, using genetically modified microorganisms.

Any microorganism is appropriate for use in the context of the invention, although certain microorganisms may be better adapted to particular culture conditions, exhibit better tolerance to end products or intermediates, or have desired endogenous enzymatic activities. Suitable microorganism include, e.g., grain negative microorganisms, grain positive microorganisms, algae, or yeast. Exemplary microorganisms include, but are not limited to, members of the genera Clostridium, Zymomonas, Escherichia, Salmonella, Rhodococcus, Pseudomonas, Bacillus, Lactobacillus, Enterococcus, Alcaligenes, Klebsiella, Paenihacillus, Arthrobacter, Corynebacterium, Brevibacterium, Pichia, Candida, Hansenula and Saccharomyces, such as Oligotropha carboxidovorans, Escherichia coli, Alcaligenes eutrophus, Bacillus licheniformis, Bacillus subtilis, Paenihacillus macerans, Rhodococcus erythropolis, Pseudomonas putida, Lactobacillus plantarum, Enterococcus faecium, Enterococcus gallinarium, Enterococcus faecalis, and Saccharomyces cerevisiae.

In various embodiments, the microorganism is modified to produce exogenous polypeptides or overproduce endogenous polypeptides having a desired activity. Alternatively or in addition, the microorganism is modified to reduce expression of endogenous polynucleotides having undesired activity. As used herein, an “exogenous” polynucleotide or polypeptide refers to any polynucleotide or polypeptide that does not originate from the cell as found in nature. A synthetic polynucleotide is “exogenous” to a cell once introduced into the cell. A polynucleotide or polypeptide that is naturally-occurring to a first host cell is exogenous to a second host cell if the polynucleotide or polypeptide is not naturally found in the second host cell.

One or more enzymatic pathways driving the conversion of a carbon source to 3-HP are optionally constructed and, if desired, competing pathways for the carbon source are minimized. For example, a recombinant microorganism expressing an exogenous glycerol dehydratase, such as the glycerol dehydratase encoded by the dhaB gene from Klebsiella pneumoniae, and an exogenous aldehyde dehydrogenase, such as ALDH2 from Homo sapiens, ALD4 from S. cerevisiae, ALDB or ALDA from E. coli, produces 3-HP from glycerol (see U.S. Pat. No. 6,852,517),

Alternatively, 3-HP is generated through one or more metabolic pathways involving pyruvate or phosphoenolpyruvate (PEP). β-alanine, for example, is a precursor for 3-HP in multiple pathways and is generated from pyruvate by pyruvate carboxylase, which mediates the conversion of pyruvate to oxaloacetate, which is converted to asparate by aspartate aminotransferase, and the aspartate is converted to β-alanine by aspartate decarboxylase. A more direct route to β-alanine involves the generation of α-alanine from pyruvate by alanine dehydrogenase, and subsequent conversion to β-alanine by, e.g., alanine 2,3-aminomutase. In this regard, a microorganism (e.g., E. coli) is engineered to express alanine 2,3-aminomutase (derived from, e.g., B. subtilis) to produce increased amounts of β-alanine for further processing. See, e.g., U.S. Pat. No. 7,309,597. In various embodiments, the microorganism comprises an exogenous or overexpressed polynucleotide encoding one or more polypeptides having any of the following enzyme activities to increase production u[3-HP: CoA transferase (mediating, e.g., the conversion of β-alanine to β-alanine-CoA and/or the conversion of 3-HP-CoA to 3-HP). β-alanyl-CoA ammonia lyase (mediating, e.g., the conversion of β-alanine-CoA to acrylyl-CoA), and 3-hydroxypropionyl-CoA dehydratase (med)ating, e.g., the conversion of acrylyl-CoA to 3-HP-CoA). Additionally or alternatively, the microorganism comprises one or more exogenous polynucleotides encoding one or more polypeptides having any of the following enzyme activities: glutamate dehydrogenase (mediating, e.g., the conversion of glutamate to 2-oxoglutarate), 3-hydroxypropionyl-CoA hydrolase (mediating, e.g., the conversion of glutamate to 2-oxoglutarate), and/or 3-hydroxyisobutyryl-CoA hydrolase (mediating, e.g., the conversion of 3-HP-CoA to 3-HP), alanine dehydrogenase (mediating, e.g., the conversion of α-alanine to β-alanine), and pyruvate-glutamate transaminase. Alternatively or in addition, the microorganism also includes an exogenous or overexpressed polynucleotide encoding 4-aminobutyrate and/or beta-alanine-2-oxoglutarate aminotransferase and/or 3-HP dehydrogenase (mediating, e.g., the conversion of malonic semialdehyde to 3-HP) and/or 3-hydroxyisobutyrate dehydrogenase (mediating, e.g., the conversion of malonic semialdehyde to 3-HP). Materials and methods for producing a recombinant microorganism producing one or more of the polypeptides described above, including polynucleotide sequences and amino acid sequences, are further described in U.S. Pat. Nos. 7,309,597 and 7,655,451.

An alternative pathway comprises microbial conversion of lactate to lactyl-CoA to acrylyl-CoA to 3-HP-CoA to 3-HP. In one example, E. coli are transformed with polynucleotides encoding a CoA transferase and a lactyl-CoA dehydratase from, e.g., Megasphaera elsdenii, and a 3-HP-CoA dehydratase from, Chloroflexus auruntiacus. Materials and methods for producing the recombinant microorganism, including additional modifications conferring an ability to produce 3-HP in a recombinant cell and amino acid and polynucleotide sequences encoding the desired enzymatic activities, are described in further detail in International Patent Publication No. WO 2002042418,

Numerous examples of recombinant organisms capable of producing 3-HP also are described in International Patent Publication No. WO 2010011874, The metabolic complex identified therein as the 3-HP Toleragenic Complex (“3HPTGC”) is modified to increase 3-HP production and/or increase tolerance to 3-HP or intermediates thereof. In this regard, in various aspects, a recombinant microorganism for producing 3-HP from a carbon source comprises a nucleic acid sequence that encodes a polypeptide with at least 90% amino acid sequence identity to any of the enzymes of any 3-HP tolerance-related or biosynthetic pathways described in International Patent Publication WO 2010011874, wherein the polypeptide has enzymatic activity and specificity effective to perform the enzymatic reaction of the respective 3-HP tolerance-related or biosynthetic pathway enzyme, and the recombinant microorganism exhibits greater 3-HP tolerance and/or 3-HP bio-production than an appropriate control microorganism lacking such nucleic acid sequence. An example of a suitable recombinant microorganism is an E. coli strain comprising a malonyl-coA reductase coding sequence from Chloroflexus aurantiacus, which converts malonyl-coA to 3-HP.

4. Direct Fermentation of Sugars to Acrylic Acid

Recombinant microorganisms also are useful for directly producing acrylic acid from a carbon source in a microorganism, i.e., without isolating a precursor such as 3-HP or lactic acid. Optionally, the microbe tolerates increased temperature or acidic environments. Mesophilic or thermophilic microorganisms, such as Thermoanaerobacterium saccharolyticum or Clostridium thermocellum, are suitable for use in the context of the invention. In various aspects, the microorganism is capable of degrading biomass, feedstock, and/or cellulose. C. thermocellum, for example, possesses the inherent ability to depolymerize and debranch cellulose and hemicelluloses. Microorganisms lacking native cellulolytic activity are optionally modified to produce one or more enzymes selected from a cellulase, a endogluconase, a exogluconase, a glucoside hydrolase, a xylanase, a xylosidase, a xyloglucanase, a mannanase, a mannosidase, a galactanase, a galactosidase, a arabinase, a arabinofuranosidase, a lignin peroxidase, a cellobiose dehydrogenase, an aryl alcohol oxidase proteinase, a nuclease, and/or a carbohydrate active enzyme (such as amylase, chitosanase, fructosidase or glycosyltransferease) to create an exogenous cellulose degradation system. Recombinant microorganisms capable of degrading biomass are further described in, e.g., International Patent Publication No. WO 2010075529. The biomass is optionally hydrolyzed before exposure to the microorganisms to reduce the structural complexity of the starting material. If desired, the carbon source is treated with heat, steam, and/or acid to degrade the material and, if the microbe cannot process cellulose, generate sugars fermentable by the microorganism.

Numerous metabolic pathways can be optimized or modified to directly produce acrylic acid from a carbon source in a cell. Many metabolic pathways are described above with respect to generating precursors of acrylic acid. If desired, the metabolic pathways described above are further modified to continue the conversion of the precursor to acrylic acid.

Several cellular process resulting in fermentation of sugar to acrylic acid involves a pyruvate intermediate. In an exemplary metabolic pathway, pyruvate is converted to lactate by, e.g., lactate dehydrogenase (E.C. 1.1.1.28), which is converted to lactoyl-CoA by, e.g., CoA transferase (E.C. 2.8.3.x), which is converted to acryloyl-CoA by, e.g., lactoyl-CoA dehydratase (E.C. 4.2.1.x), which is finally converted to acrylate by, e.g., CoA transferase (E.C. 2.8.3.x). In an alternative pathway, pyruvate is converted to L-α-alanine by. e.g., alanine aminotransferase (E.C. 2.6.1.2), which is converted to L-aspartate by, e.g., aspartate 4-decarboxylase (E.C. 4.1.1.12), which is converted to β-alanine by, e.g., aspartate 1-decarboxylase (E.C. 4.1.1.11), which is converted to β-alanyl-CoA by, e.g., β-alanyl-CoA:ammonia lyase (E.C. 4.3.1.6), which is converted to acryloyl-CoA by, e.g., β-alanyl-CoA:ammonia lyase, which is finally converted to acrylate by, e.g., CoA transferase (E.C. 2.8.3.x). In yet another alternative pathway, pyruvate is converted to lactate by, e.g., lactate dehydrogenase (E.C. 1.1.1.28), and lactate is converted directly to acrylate, by, e.g., lactate dehydratase (E.C. 4.2.1.x). These exemplary metabolic pathways are further described in, e.g., International Patent Publication No. WO 2010105194.

The invention includes an absorbent article (and methods of making an absorbent article and/or elements thereof) constructed from a polymer produced by a recombinant microorganism comprising one or more non-native (or exogenous) polynucleotides encoding for one or more exogenous enzymes that function to convert pyruvate into acrylate via the metabolic pathways described above (e.g., a metabolic pathway involving the conversion pyruvate to lactate, the conversion lactate to acrylate, the conversion lactoyl-CoA to acryloyl-CoA, the conversion acryloyl-CoA to acrylate, the conversion pyruvate to L-α-alanine, the conversion L-α-alanine to L-aspartate, the conversion L-aspartate to β-alanine, the conversion β-alanyl-CoA to acryloyl-CoA, and/or the conversion acryloyl-CoA to acrylate). Optionally, the recombinant microorganism is modified to partially, substantially, or completely silence (i.e., down-regulate) one or more native enzymes involved in metabolic pathways that compete with the conversion of pyruvate to acrylic acid. For example, redirecting carbon flow from acetate and ethanol towards lactate improves acrylate yield. Thus, in various aspects, the recombinant microorganism is modified to reduce or ablate the production or activity of, e.g., acetate kinase (ack), phosphotransacetylase (pta), alcohol dehydrogenase (adh), and/or pyruvate-formate-lyase (pfl). Materials and methods for generating recombinant microbes producing exogenous polypeptides having the activity described above (or overexpression endogenous polypeptides having these activities), as well as use of the microbes to produce acrylic acid, arc further described in International Patent Publication No. WO 2010105194.

An alternative metabolic pathway initiates with glucose metabolism to phosphoenol pyruvate (PEP) via glycolysis. PEP reacts with carbon dioxide (via PEP carboxylase or PEP carboxykinase) to yield fumarate, which is decarboxylated to yield acrylic acid. The invention includes producing acrylic acid using a recombinant microorganism producing an exogenous decarboxylase (or overproducing an endogenous decarboxylase) that drives conversion of fumarate to acrylic acid. To increase carbon flow through the pathway, the recombinant microorganism optionally includes one or more mutations in coding sequences encoding fumarate reductase (FRD) and/or alcohol dehydrogenase (ADHEr) and/or lactate dehydrogenase (LDH). Alternatively, the microbe exhibit reduced production or activity of FRD, ADHEr, or LDH is co-cultured with a second microbe producing a decarboxylase. In various aspects, the microbe further (or alternatively) comprises modifications linking the production of elevated levels of fumaric acid with the exponential growth phase, which, in some instances, minimizes negative selective pressures on the microbe. For example, in E. coil, fumaric acid production is coupled to exponential growth by disrupting, e.g., fumAB or C genes (b1611, b1612 and/or b4122), gnd (b2029), and/or purU (b1232). The fumarate pathway and materials and methods for producing a microbial strains that yield acrylic acid from fumarate are further described in International Patent Publication Nos. WO 2009155382 and WO 2009045637.

Methods of directly fermenting sugar to acrylic acid also are described in, e. International Patent Publication Nos. WO 20090011591 and 2008041840,

### 5. Metathesis

In some variations of the invention, acrylic acid is generated via metathesis of precursor compounds. In one embodiment, acrylic acid is generated via metathesis using crotonic acid as a starting material. International Patent Publication No. WO 2011002284, for instance, describes methods of synthesizing at least two different vinylic monomers, at least one of which is an acrylic compound, via cross metathesis of a monoconjugated alkene-1-carboxylic compound (e.g., crotonic acid) with a C2-C4 alkene (e.g., ethylene). Olefin cross metathesis is carried out in the presence of a catalyst, such as metal carbene complexes of tungsten, molybdenum, rhenium, chromium, osmium and ruthenium. Grubbs 1st and 2nd generation catalysts, and Hoveyda-Grubbs analogs thereof are examples of suitable catalysts. Additional examples of metathesis catalysts and methods of use are described in International Patent Publication No. WO 2011002284 (see, e.g., Table 1 illustrating CAS 172222-30-9, CAS 203714-71-0. CAS 301224-40-8, CAS 477218-66-9, and CAS 801219-49-7), and the Handbook of Metathesis, R. H. Grubbs Ed. Wiley-VCH, Weinheim (2003).

Crotonic acid for use as an intermediate is obtained from any source. Optionally, the crotonic acid is obtained via fermentation. See, e.g., International Patent Publication No. WO 2009/046828. Alternatively or in addition, crotonic acid is generated via thermolysis of a polyhydroxyalkanoate (e.g., a polyhydroxyalkanoate comprising 3-hydroxypropionate monomers, such as poly 3-hydroxypropionate). U.S. Pat. Nos. 7,166,743 and 6,897,338, for instance, describe methods of making alkenoic acids (including crotonic acid) from polyhydroxyalkanoates, such as polyhydroxybutyrate (PHB). In various embodiments, the PHA is a copolymer, such as poly 3-hydroxybutyrate-co-3-hydroxyvalerate or a PHA comprising 3-hydroxypropionate monomers in combination with another PHA building block. The method generally comprises heating a PHA to a temperature of at least about 100° C. (e.g., at least about 150° C., at least about 200° C., at least about 250° C., or at least about 300° C.). Generally, the PHA is heated at atmospheric pressure, although elevated pressure also is appropriate in various embodiments.

Alternatively, fumaric acid, such as fumaric acid produced by a microorganism as described in International Patent Publication Nos. WO 2009155382 and WO 2009045637, is optionally subjected to cross-metathesis transformation in an integrated process for generating acrylic acid. In an exemplary embodiment, fumaric acid is reacted with ethylene in the presence of a cross-metathesis transformation catalyst, such as a ruthenium catalyst (e.g., a ruthenium catalyst bearing an N-heterocyclic carbine ligand). Process conditions for cross-metathesis of fumaric acid are further described in International Patent Publication No. WO 2009045637.

Russian Patent Publication No. 2326733 provides a metathesis catalyst composition comprising a catalyst of olefin metathesis and a phenol derivative at a proportion of 1 mol catalyst to 200-1500 mol phenol derivative. In another catalytic composition, the olefin catalyst is combined with an alcohol derivative at a proportion of 1 mol catalyst to 200-1500 mol alcohol derivative. In another modification, a quinine or its derivative is present with the metathesis catalyst. The catalyst composition is useful for catalyzing metathesis of, e.g., a dialkyl maleate and/or maleate ester with ethylene. Catalyst compositions for obtaining acrylic acid ethers via metathesis reaction of dialkylmaleates also are described in International Patent Publication No. WO 2008024023.

6. Propanal or Propiolactone

Glucose derived from a renewable resource (e.g., via enzymatic hydrolysis of corn starch obtained from the renewable resource of corn) may be converted into acrylic acid by a multistep reaction pathway. In one reaction pathway, glucose is fermented to yield ethanol, which is dehydrated to yield ethylene. At this point, ethylene may be polymerized to form polyethylene. Alternatively, ethylene may be converted into propionaldehyde by hydroformylation of ethylene using carbon monoxide and hydrogen in the presence of a catalyst such as cobalt octacarbonyl or a rhodium complex. Propan-1-ol is formed by catalytic hydrogenation of propionaldehyde in the presence of a catalyst such as sodium borohydride and lithium aluminum hydride. Propan-1-ol alternatively is dehydrated in an acid catalyzed reaction to yield propylene. At this point, propylene may be polymerized to form polypropylene. However, propylene may be converted into acrolein by catalytic vapor phase oxidation. Acrolein may then be catalytically oxidized to form acrylic acid in the presence of a molybdenum-vanadium catalyst.

In another process for producing acrylic acid, propiolactone is reacted with hot phosphoric acid at elevated temperatures, e.g., about 140° C. to about 180° C. (such as about 170° C.), at a pressure of about 20 mm to about 200 mm (e.g., about 100 mm) to form acrylic acid vapors. Next, vapors of acrylic acid are maintained in super-heated condition and mixed with an acrylic acid polymerization inhibitor (e.g., mono methyl ether of hydroquinone). The acrylic acid is then condensed, optionally at a temperature of about 15° C. This process is described in further detail in U.S. Pat. Nos. 3,462,484 and 3,176,042.

U.S. Pat. No. 6,852,865 describes catalysts useful for carbonylation of 3- and 4-membered rings, such as propiolactone. For example, a 3-member ring compound (such as ethylene oxide) is reacted with carbon monoxide in the presence of a catalytically effective amount of a catalyst having the general formula [Lewis acid]²⁺, {[QM(CO)ₓ]^{w-}}_{y} where Q is any ligand and need not be present. M is a transition metal selected from the group consisting of Groups 4, 5, 6, 7, 8, 9 and ID of the periodic table of elements, z is the valence of the Lewis acid and ranges from 1 to 6, w is the charge of the metal carbonyl and ranges from 1 to 4 and y is a number such that w times y equals z, and x is a number such as to provide a stable anionic metal carbonyl for, {[QM(CO)ₓ]^{w-}}_{y}, to form a 4-member ring, such as propiolactone. An exemplary catalyst is catalyst G (0.2 Mn DME) disclosed in U.S. Pat. No. 6,852,865, herein incorporated by reference in its entirety, which also provides the following exemplary conditions for using catalyst G. A 100 mL Parr reactor is dried at 90° C., under vacuum. The reactor is cooled to −35° C. for at least 1.5 hours and equipped with a small test-tube and magnetic stir bar. The test tube is charged with 0.500 mL of ethylene oxide and catalyst G. The reactor is pressurized, placed in a preheated oil bath and the reactor is stirred at 50° C. for 1 hour. Next, the reactor is cooled in a bath of dry ice/acetone until the pressure reaches a minimum and is then slowly vented. One of ordinary skill has the requisite skill and knowledge to apply the materials and methods disclosed in U.S. Pat. No. 6,852,865 to generate acrylic acid. Thus, in one aspect of the invention, an element of an absorbent article is produced by converting ethylene oxide to propiolactone via carbonylation, and the resulting propiolactone is converted to acrylic acid.

An additional process for generating acrylic acid comprises reacting an epoxide (e.,., ethylene oxide), a solvent (e.g., sulfolane), a carbonylation catalyst (e.g., a metal carbonyl compound), and carbon monoxide to produce a reaction product stream comprising a beta-lactone (e.g., propriolactone). The beta-lactone is then separated from the product stream and treated with an acid catalyst to form acrylic acid. Materials and methods for generating propriolactone are further described in International Patent Publication No. WO 20100118128.

7. Other

Acrylic acid also may be obtained from a polyhydroxyalkanoate (PHA), such as a PHA comprising 3-hydroxypropionate monomers (e.g., poly 3-hydroxypropionate or a PHA copolymer comprising 3-hydroxypropionate). For example, as described above. PHA, such as PHB polymer, may be converted to crotonic acid via thermolysis of the polymer, and the crotonic acid intermediate is subsequently transformed to acrylic acid and/or propylene via, e.g., metathesis. Alternatively, acrylic acid is generated directly from a PHA via thermolysis, as described in U.S. Pat. Nos. 6,897,338 and 7,166,743, incorporated by reference in their entirety and particularly with respect to methods of generating acrylic acid from PHA.

Carboxylation of ethylene and carbon dioxide also produces acrylic acid. For example. Bernskoetter et al, Organometallics, 30, 520-527 (2011) describes a transition metal catalyst, tridentate phosphine molybdenum(0) complex, that couples ethylene and carbon dioxide to form acrylate. Nickel also mediates ethylene and carbon dioxide, and formation of methyl acrylate via methylation of nickelalactones has been described in. e.g., Bruckmeier et al., Organometallics, 29, 2199-2202 (2010). In one aspect of the invention, the absorbent article element comprises a synthetic polymer generated by converting ethylene to acrylic acid via reaction with carbon dioxide.

Carbon dioxide used in any of the routes described herein is obtained from any source, and is optionally obtained from a renewable resource. In various aspects, the carbon dioxide is, e.g., a byproduct of sugar fermentation, a product of sugar metabolism, collected from the burning of plant material, isolated from the atmosphere, or recycled from other chemical reactions.

Propionic acid is an intermediate for both acrylic acid and propylene, which may be polymerized to polypropylene or used as an intermediate in reaction schemes for generating acrylic acid. A number of reactions are known in the art to convert propionic acid to acrylic acid, several of which are described herein. An exemplary method entails dehydrogenation of propionic acid using a catalyst, such as an iron phosphate, molybdenum phosphate, or vanadium phosphate catalyst, as described in, e.g., Ai, Kinetics and Catalysis, 44(2), 198-201 (2003). From Kinetika i Kataliz, 44(2), 2)4-217 (2003). In some variations, the catalyst is an iron phosphate catalyst comprising molybdenum.

While the above reaction pathways yield acrylic acid, a skilled artisan will appreciate that acrylic acid may be readily converted into an ester (e.g., methyl acrylate, ethyl acrylate, etc.) or salt.

### B. Ethylene and Propylene

Olefins, such as ethylene and propylene, also are capable of being derived from renewable resources. Alcohols are employed as intermediates for, e.g., propylene, in a variety of reaction schemes. Alcohols may be directly derived from biomass or derived from a renewable resource via one or more other intermediates. For example, methanol, optionally derived from fermentation of biomass, may be converted to ethylene and or propylene, which are both suitable monomeric compounds for preparing the synthetic polymer, as described in U.S. Pat. Nos. 4,296,266 and 4,083,889.

U.S. Pat. No. 4,296,266 describes a process for the manufacture of lower olefins (e.g., 2-4C olefins) from methanol and/or dimethyl ether using a manganese-containing aluminum silicate catalyst washed with EDTA or a tartaric acid solution with a pH of 3 to 7. Examples of possible aluminum silicates are the customary, amorphous acid cracking catalysts, which in general contain about 13% to about 25% by weight of aluminum oxide and about 75% to about 87% by weight of silica. Naturally occurring or synthetic crystalline aluminum silicates are also suitable, such as those which are known, for example, by names such as faujasites, zeolites, chabasites, analcime, gismondite, gmelinite, natrolite, mordenites and erionites, or generally as molecular sieves.

U.S. Pat. No. 4,083,889 describes a process for manufacturing ethylene by catalytic conversion of a methanol feed in the presence of steam or water diluent. The process is optionally independent of petroleum feedstocks, as the methanol feed may be manufactured from synthesis gas, i.e., a mixture of CO and H₂. The presence of the steam diluent in the process induces sustained high catalytic activity with high selectivity for the formation of ethylene while retaining high conversion levels. Suitable catalysts include zeolite catalysts, such as HZSM-5, a crystalline aluminosilicate zeolite, and the conversion is conducted at relatively low temperature, e.g., from about 600° F. to about 750° F. The hydrocarbon conversion product is an olefin-rich hydrocarbon mixture, containing a high concentration of ethylene, i.e. at least 18 wt. %.

Ethanol or propanol (e.g., n-propanol or i-propanol) derived from fermentation of a renewable resource may be converted into ethylene or propylene via dehydration as described in, e.g., U.S. Pat. No. 4,423,270. U.S. Pat. No. 4,423,270 provides a process for the catalytic dehydration of ethanol vapor to ethylene using a supported organophosphorus catalyst. Similarly, propanol or isopropanol derived from a renewable resource can be dehydrated to yield the monomeric compound of propylene as exemplified in U.S. Pat. No. 5,475,183. Propanol is a major constituent of fusel oil, a by-product formed from certain amino acids when potatoes or grains are fermented to produce ethanol. Suitable catalysts include a γ-alumina catalyst which contains 0.3% by weight or less of impurities in total (excluding SiO₂), preferably 0.1% by weight or less. Sulfur content in the impurities may be 0.2% by weight or less, preferably 0.1% by weight or less, more preferably 0.06% by weight or less, calculated in terms of SO₄⁻. Sodium content in the impurities may be 0.05% by weight or less, preferably 0.03% or less, calculated in terms of Na₂O. When the sum total of impurities in the γ-alumina catalyst are restricted within the aforementioned ranges, catalyst conversion into the α-form is minimized and, therefore, catalytic activity does not decrease after use in the dehydration reaction for a prolonged period of time at a temperature of from about 150° C. to about 500° C. under pressure. In addition, a γ-alumina catalyst with reduced sodium content improves yield of the dehydration reaction. Such process produces lower olefins from lower alcohols with high yield and high selectivity for a prolonged period of time without reducing catalytic activity.

Charcoal derived from biomass can be used to create syngas (i.e., CO+H₂) from which hydrocarbons such as ethane and propane can be prepared (Fischer-Tropsch Process). Ethane and propane can he dehydrogenated to yield the monomeric compounds of ethylene and propylene,

Additional renewable processes for producing propylene include contacting ethylene with a metathesis catalyst to form a metathesis product stream comprising propylene. Examples of these processes include those described in published European Patent Application No. 1953129A1 or counterpart U.S. Patent Application Publication No. 20080312485, U.S. Patent Application Publication No. 20100168487, U.S. Pat. No. 4,242,531, and U.S. Pat. No. 6,586,649.

U.S. Patent Application Publication No. 20080312485 describes a reaction scheme for converting ethanol to propylene via metathesis while minimizing catalyst deterioration caused by water present in ethanol derived from biomass. First, ethanol (e.g., ethanol obtained from biomass)'s dehydrated to form ethylene. Ethylene is then separated from the water byproduct of the dehydration reaction and purified by adsorption. Suitable adsorbents depend on the impurities to be removed and include, but are not limited to, alumina, magnesium oxide or a mixture thereof, and zeolite. A metathesis reaction is then carried out with the resulting ethylene and n-butene. N-butene is obtained by any method, including methods described in detail in U.S. Patent Application Publication No. 20080312485. Butene may be obtained, for example, by dehydration of butanol. Prior to use in the metathesis reaction, water and polar substances are removed from n-butene. When carrying out the metathesis reaction, any molar ratio of ethylene and n-butene may be used. In some variations, an excessive amount of ethylene is utilized. The ratio of ethylene to n-butene is preferably about 0.1 to about 50, such as about 0.5 to about 5. A metathesis catalyst optionally contains at least one kind of metal including, but not limited to, tungsten, molybdenum, rhenium, niobium, tantalum, vanadium, ruthenium, rhodium, iridium, osmium, and nickel. The reaction scheme is further described in U.S. Patent Application Publication No. 20080312485. Thus, in one aspect of the invention, the method of producing an element of the absorbent article comprises converting butanol to butene via, e.g., dehydration, and converting the resulting butene to propylene via, e.g., metathesis.

Another method of propylene production by metathesis is described in U.S. Patent Application Publication No. 20100168487. The process described in U.S. Patent Application Publication No. 20100168487 comprises reacting a feed stream comprising isobutene in the presence of a skeletal isomerization catalyst to obtain an isomerized stream comprising C₄ olefins, and reacting the isomerized stream with ethylene in the presence of a metathesis catalyst to form a metathesis product stream comprising propylene, C₄ olefins, and C₅ and higher olefins. The step of reacting the isomerized stream with ethylene, i.e. the metathesis reaction, is performed at an equal or lower pressure than the step of reacting the feed stream, i.e. the skeletal isomerization step. In some aspects, the pressure of the metathesis reaction is conducted at about 15 psig to about 100 psig. In particular aspects, the metathesis reaction pressure is about 20 psig to about 60 psig. One advantage of the process is that it does not require cooling the isomerized stream, pressurizing it, then heating it up again to a temperature suitable for the metathesis reaction and, therefore, the process saves energy.

Yet another method of producing propylene via metathesis is described in U.S. Pat. No. 4,242,531, which describes methods for olefin dimerization using a loop reactor. In one aspect, all or part of the desired product is removed as a vapor by flashing the reactor effluent in a flashing zone, optionally within the loop of the loop reactor. Unconverted ethylene may be recovered for recycling in an absorber utilizing a heavies product stream, which is a product of the process, as the absorbent. A liquid gas absorber contractor within the loop reactor also may be used to concentrate the olefin. The process and apparatus described in U.S. Pat. No. 4,242,531 are suitable for use with any appropriate dimerization catalyst, such as any hydrocarbon-soluble nickel compound, alkyl aluminum halide, or a mixture thereof, e.g., tri-n-butylphosphine nickel dichloride mixed with ethyl aluminum dichloride or bis(tri-n-butyl-phosphine)dichloronickel. The ethylene in the feed gas is dimerized to butenes which are much more easily recovered and separated from the gases than ethylene. Additionally, removal of most of the major product as a vapor allows retention of the catalyst in the reactor for a longer period of time, thereby improving catalyst productivity.

Butene metathesis, described in, e.g., U.S. Pat. No. 6,586,649, also is contemplated as a means for producing propylene. The process includes contacting a starting material containing butene with a catalyst under conditions suitable for forming propylene. The catalyst includes, for example, a catalytic amount of at least one metal oxide selected from oxides of the transition metals. Catalysts include, but are not limited to, oxides of molybdenum, oxides of rhenium, oxides of tungsten, and mixtures thereof. The catalyst is optionally supported on a solid ceramic support of silica, alumina, titania, zirconia or mixtures thereof, wherein the transition metal oxide forms about 1%, to about 30% of the total heterogeneous catalyst mass. The metathesis reaction is optionally performed at a temperature of about 300° C. to about 600° C. and a pressure of about 1-20 atmospheres. Hydrocarbons other than propylene produced by the metathesis reaction are separated from the product and recycled back into contact with the catalyst to increase the yield.

Renewable methods for producing propanol for aspects of the invention are provided in U.S. Patent Application Publication No. 20090246842, published European Patent Application No. 2184354, U.S. Patent Application Publication Nos, 20100203604 and 20100209986, and International Patent Publication No. WO 2010/127303.

U.S. Patent Application Publication No. 20090246842 describes methods and compositions for the production of propanol, such as propanol from bio-based precursors. More specifically, U.S. Patent Application Publication 20090246842 describes engineered microorganisms that produce isopropanol at high yield by biochemically converting a carbon source to acetyl-CoA and converting acetyl-CoA to isopropanol. At least one enzyme of the fermentative pathway is heterologous to the microorganism, and the host cells are cultured under conditions suitable for producing isopropanol. In various aspects, the microbe is engineered to produce an exogenous protein (or overproduce an endogenous protein) catalyzing one or more of the following conversions: Acetyl-CoA to Acetate and CoA (mediated by, e.g., phosphate acetyltransferase and acetate kinase); Acetyl-CoA to Acetoacetyl-CoA and CoA (mediated by, e.g., acety)-CoA-acetyltransferase); Acetoacetyl-CoA and Acetate to Acetoacetate and Acetyl-CoA (mediated by, e.g., acetoacetyl-CoA:acetate/butyrate coenzyme-A transferase); Acetoacetyl-CoA+H₂O Acetoacetate+CoA (mediated by, e.g., acetoacetyl-CoA hydrolase); (v) Acetoacetate to Acetone and CO₂ (media(ed by, e.g., acetoacetate decarboxylase); and (vi) Acetone and NAD(P)H and H⁺ to Isopropanol and NAD(P)+(mediated by, e.g., secondary alcohol dehydrogenase). Materials and methods for producing the recombinant microbe, as well as methods of using the recombinant microbe to produce isopropanol, are further described in U.S. Patent Application Publication No. 20090246842.

U.S. Patent Application Publication No. 20100203604 also describes recombinant microorganisms comprising an engineered metabolic pathway for producing isopropanol via an acetoacetate-CoA intermediate. The recombinant microorganism, e.g., an aerobic bacterium or a facultative anaerobic bacterium, comprises an exogenous (or overexpressed) polynucleotide encoding an enzyme having acetyl-CoA acetyltransferase activity, an exogenous (or overexpressed) polynucleotide encoding an enzyme having acetoacetyl-CoA:acetate CoA-transferase activity, an exogenous (or overexpressed) polynucleotide encoding an enzyme having acetoacetate decarboxylase activity, and an exogenous (or overexpressed) polynucleotide encoding an enzyme having isopropanol dehydrogenase activity. Examples of suitable recombinant microorganisms include, but are not limited to Escherichia coil, Coryneform bacteria. Streptococcus, Staphylococcus, Enterococcus, Bacillus and Streptomyces; fungus cells, such as Aspergillus; and yeast cells, such as baker's yeast and Pichia pastoris. These organisms efficiently produce high levels of isopropanol because they are capable of rapid proliferation under aerobic conditions. To produce isopropanol, the recombinant microorganism is cultured in a medium containing saccharides, and isopropanol is collected from the culture. Materials and methods for producing propanol using recombinant microbes, including polynucleotide sequences encoding the above-mentioned enzymes, are further described in U.S. Patent Application Publication No. 20100203604.

Published European Patent Application No. 2184354, also published as U.S. Patent Application Publication No. 20100311135, describes bacterium producing an acetoacetate decarboxylase (E.C. 4.1.1.4), an isopropyl alcohol dehydrogenase (E.C. 1.1.1.80), a CoA transferase (E.C. 2.8.3.8) and a thiolase (E.C. 2.3.1.9). The bacterium is capable of generating isopropyl alcohol from a plant-derived material. The enzymes are not native to the host, or are produced at a higher level than achieved in nature. The microbe is cultured in the presence of a plant material (e.g., root, stem, stalk, branch, leaf, flower, seed, degradation products of any of the foregoing, or carbon sources derived from any of the foregoing, such as starch, glucose, fructose, sucrose, xylose, arabinose, glycerin and fatty acids) under conditions suitable for producing isopropanol. Isopropanol is collected from the culture medium by various means, including a production apparatus comprising (i) a culturing unit; (ii) a gas-supplying unit connected to the culturing unit and opening at a position in the mixture contained in the culturing unit; (iii) a capture unit containing at least the capture liquid which captures isopropyl alcohol; and (iv) a connecting unit which connects the culturing unit with the capture unit and allows isopropyl alcohol evaporated in the culturing unit to move to the capture unit.

An alternative method of generating alcohol precursors to absorbent polymers is described in U.S. Patent Application Publication No. 20100209986, which discloses use of metabolically-modified microorganisms for producing, e.g., isobutanol, 1-butanol, 1-propanol. 2-methyl-1-butanol, 3-methyl-1-butanol or 2-phenylethanol, from a 2-keto acid (e.g., 2-ketobutyrate), a metabolite generated in an organism's native amino acid pathway. In one aspect, the microorganism comprises an exogenous polynucleotide (or overexpressed polynucleotide) encoding an enzyme that catalyzes the condensation of pyruvate and acetyl-coA. In this regard, the microorganism comprises an exogenous polynucleotide (or overexpressed polynucleotide) encoding 2-keto-acid decarboxylase (e.g., pdc, pdc1, pdc5, pdc6, uro10, thi3, kivd, or kdcA) and/or an acetohydroxy acid synthase and/or an acetohydroxy acid isomeroreductase and/or a dihydroxy-acid dehydratase and/or an alcohol dehydrogenase and/or a citramalate synthase (cimA). Alternatively, the microorganism comprises an exogenous polynucleotide (or overexpressed polynucleotide) encoding a citramalate synthase in combination with an exogenous polynucleotide (or overexpressed polynucleotide) encoding alpha-isopropylmalate synthase and/or beta-isopropylmalate dehydrogenase and/or isopropylmalate isomerase and/or threonine dehydratase. Optionally, the recombinant microorganism comprises one or more deletions or knockouts in a gene encoding an enzyme that catalyzes the conversion of acetyl-coA to ethanol; catalyzes the conversion of pyruvate to lactate: catalyzes the conversion of fumarate to succinate; catalyzes the conversion of acetyl-coA and phosphate to CoA and acetyl phosphate; catalyzes the conversion of acetyl-CoA and formate to CoA and pyruvate; catalyzes the condensation of the acetyl group of acetyl-CoA with 3-methyl-2-oxobutanoate (2-oxoisovalerate); catalyzes isomerization between 2-isopropylmalate and 3-isopropylmalate; catalyzes the conversion of alpha-keto acid to branched chain amino acids; catalyzes synthesis of phenylalanine, tyrosine, aspartate, or leucine; catalyzes the conversion of pyruvate to acetyl-CoA; catalyzes the formation of branched chain amino acids; catalyzes the formation of alpha-ketobutyrate from threonine; catalyzes the first step in methionine biosynthesis; and/or catalyzes the catabolism of threonine. Modifying an organism's native amino acid pathways to produce higher alcohols avoids the difficulty of expressing a large set of exogenous genes in the microbe and minimizes the possible accumulation of toxic intermediates.

An additional metabolic pathway for producing isopropanol comprises 4-hydroxybutyryl-CoA as a precursor. International Patent Publication No. WO 2010127303, also published as U.S. Patent Application Publication No. 20100323418, provides non-naturally occurring microbial organisms comprising one or more exogenous nucleic acid(s) encoding an activity mediating the conversion of 4-hydroxybutyryl-CoA to crotonoyl-CoA, the hydration of crotonoyl-CoA to form 3-hydroxybutyryl-CoA, the oxidation of 3-hydroxybutyryl-CoA to form acetoacetyl-CoA, and/or the conversion of acetoacetyl-CoA to isopropanol. In this regard, the recombinant microorganism comprises one or more exogenous (or overexpressed) nucleic acid(s) encoding any one or more of the following: a 4-hydroxybutyryl-CoA dehydratase, a crotonase, a 3-hydroxybutyryl-CoA dehydrogenase, an acetoacetyl-CoA synthetase, an acetyl-CoA:acetoacetate-CoA transferase, an acetoacetyl-CoA hydrolase, an acetoacetate decarboxylase, and an acetone reductase, U.S. Patent Application Publication No. 20100323418 further describes a non-naturally occurring microbial organism having an engineered n-butanol pathway or isobutanol pathway. The microorganisms are cultured in the presence of a carbon source, such as glucose, for a sufficient time and under suitable conditions for producing alcohol. Materials and methods for producing the recombinant microorganisms, as well as fermentation conditions for producing propanol, are further described in U.S. Patent Application Publication No. 2010/0323418.

Alcohol precursors (e.g. propanol) also may be derived from a carboxylic acid or its ester propionic acid or its ester). One method of deriving carboxylic acid or its ester to alcohol is described in Chinese Patent No. 1974510, which is hereby incorporated by reference. Briefly, a mixture of carboxylic acid (21%-92% wt.), catalyst (1%-21% wt.), and solvent (5%-73% wt.) is reacted with high purity hydrogen at 100-200° C. at a pressure not lower than 1.0 MPa, preferably 3.0-7.0 MP, while stirring at 500-1000 rpm. The alcohol is then collected after cooling. A non-limiting example of a suitable catalyst for the reaction is prepared by adding RuCl₂(TPPS)₃, RuCl₃ or ruthenium acaetylacetonate, to a chloride or nitrate solution of rhodium, platinum, palladium, iron, tin, zinc, nickel, or cobalt (4%-60% wt.), placing the mixture in a water bath (45-55°), dropping ammonia water solution (23%-55% wt.) and zirconium hydroxide (17%-41% wt.) simultaneously into the mixture to obtain precipitate, stirring the mixture over night, and collecting the resulting catalyst.

Fermentation-based methods for converting biomass to intermediates or synthetic polymer of the invention also are described in, e.g., U.S. Pat. No, 4,698,304, U.S. Patent Application Publication No. 20070031919, International Patent Publication No. WO 2010/001078, and European Patent No. 350355.

U.S. Pat. No. 4,698,304 discloses methods for producing mixtures of saturated or unsaturated C₂-C₅ hydrocarbons by aerobically cultivating a microorganism in a water-containing medium, and recovering the hydrocarbon mixtures from the liquid phase or/and gaseous ambience of the medium. A wide variety of genera is suitable for use in the method, including (but not limited to) fungi (such as Saprolegenia, Phytophthola, Mucor, Rhizopus, Absidia, Mortierella, Cunninghamella, Taphrina, Monascus, Nectria, Gibberella, Chaetomiuin, Neurospora, Geotrichum, Tricoderma, Aspergillus, Penicillium, Paecilomyces, Glvocladium, Sporotrichum, Microsporum, Trichophyton, Cladosporium, Syncephalastrum, Phycomyces, or Eupenicillium), yeast (such as Endomyces, Shizosaccharomyces, Saccharomyces, Pichia, Hansenula, Dabaryomyces, Saccharomycopsis, Rhodotorula, Sporobolomyces, Cryptococcus, Candida, or Brettanotnyces), bacteria (such as Bacillus, Brevibacterium, Corynebacterium, Flavobacterium, Klebsiella, Micrococcus, Mycoplana, Paracoccus, Proteus, Pseudomonas, Salmonella, Serratia, or Acetobacter), and Actinomycetes (such as Streptomyces, Actinomyces, or Intatsporangium). Industrial wastes and various biomasses are optionally utilized as nutrient sources in the cultivation. Optionally, vitamins and/or amino acids (L-leucine, L-isoleucine, L-methionine. and/or L-cysteine) are added to the culture to enhance strain growth or improve hydrocarbon yields. Alcohol production is accomplished under mild conditions, including relatively low temperature and low pressure, and the impurity gases are mostly carbon dioxide. As a result, hydrocarbon mixtures produced by the method described U.S. Pat. No. 4,698,304 are easy to collect, concentrate, and recover.

U.S. Patent Application Publication No. 20070031919 provides additional methods of producing polymer precursors from biomass via fermentation. Biomass is pretreated, at relatively high concentration, with a low concentration of ammonia relative to the dry weight of biomass. Following pretreatment, the biomass is treated with a saccharification enzyme consortium to produce fermentable sugars, e.g. oligosaccharides and monosaccharides, that can be used as a carbon source by a microorganism in a fermentation process. The sugars are then contacted with a microbe that ferments the sugars and to produce a target chemical. In one embodiment, the method comprises (a) contacting biomass with an aqueous solution comprising ammonia at a concentration of less than about 12 weight percent relative to dry weight of biomass but at least sufficient to maintain alkaline pH of the biomass-aqueous ammonia mixture. The dry weight of biomass is optionally at a high solids concentration of at least about 15 weight percent relative to the weight of the biomass-aqueous ammonia mixture. The method further comprises (b) contacting the product of step (a) with a saccharification enzyme consortium under suitable conditions to produce fermentable sugars. The saccharification enzyme consortium optionally comprises at least one enzyme selected from the following: cellulose-hydrolyzing glycosidases, hemicellulose-hydrolyzing glycosidases. starch-hydrolyzing glycosidases, peptidases, lipases, ligninases, feruloyl esterases, cellulases, endoglucanases, exoglucanases, cellobiohydrolases, β-glucosidases, xylanases, endoxylanases, exoxylariases, β-xylosidases, arabinoxylanases, mannases, galactases, pectinases, glucuronidases, amylases, α-amylases, β-amylases, glucoamylases, α-glucosidases, and isoamylases. The method further comprises (c) contacting the product of step (b) with at least one microbe able to ferment the sugars to produce the target chemical under suitable fermentation conditions. Any microbe that uses fermentable sugars may be used to make the target chemical(s), and examples of microbes include (but are not limited to) wild type, mutant, or recombinant Escherichia, Zymoinonas, Candida, Sacearomyces, Pichia, Streptomyces, Bacillus, Lactobacillus and Clostridium. In various embodiments, the biomass is switchgrass, waste paper, sludge from paper manufacture, corn grain, corn cobs, corn husks, corn stover, grasses, wheat, wheat straw, hay, barley, barley straw, rice straw, sugar cane bagasse. sorghum, soy, components obtained from processing of grains, trees, branches, roots, leaves, wood chips, sawdust, shrubs and bushes, vegetables, fruits, flowers and/or animal manure.

International Patent Publication No. WO 2010/001078 describes processes for biologically producing alkenes by enzymatic decarboxylation of 3-hydroxyalkanoic acids. More particularly, terminal alkenes are produced by enzymatic decarboxylation of 3-hydroxyalkanoate molecules using, e.g., an MDP decarboxylase (E.C. 4.1.1.33). In one aspect, the process comprises a 3-phospho-hydroxyalkanoate intermediate, and the enzyme comprises both decarboxylase and phosphorylase activity. The method can be implemented in cell-free systems or by using a microorganism that produces the decarboxylase (endogenously or exogenously). Materials and methods for producing terminal alkenes are further described in International Patent Publication No. WO 2010/001078.

European Patent No. 350355 describes a method of generating butyric acid via fermentation using at least one strain of Clostridium, such as Clostridium tyrobutyricum IFP 923. In various embodiments of the method, the bacterial strain is provided a sugar substrate (e.g., a monosaccharide such as glucose) at an initial concentration of from about 1-80 g of sugar per liter of initial reaction medium. The sugar substrate concentration in the medium is allowed to decrease by at least 20% of its initial value, reaching a concentration of 30 g or less of sugar substrate per liter of initial reaction medium. The bacterial strain is then provided additional sugar substrate at a rate of between 0.01-25 g per liter of initial reaction medium per hour. The residual concentration of the sugar substrate during the feeding period is not more than 80 g per liter of initial reaction medium. Butyric acid is then recovered, optionally by distillation. Additional catalytic methods of producing propylene or precursors thereof are described in, e.g., U.S. Patent Application Publication Nos. 20100069589, 20090326293, 20060020155, and 20100069691; U.S. Pat. No. 7,102,048; European Patent Application No. 2108635; Japanese Patent Application No. 5213778; and International Patent Publication Nos. WO 2010/096812, WO 2008/103480, and WO 2009/073938.

Ethylene and propylene also is obtained via fermentation of propanoic and butyric acids, respectively, using the materials and methods described in International Patent Publication No. WO 2011066634. International Patent Publication No. WO 2011066634 describes a process whereby ethylene or propylene is produced by anodic decarboxylation of carboxylic acids, e.g., propionic acid or butyric acid. The propionic acid or butyric acid is produced via biofermentation using, e.g., Propionthacterium for propanoic acid or Clostridium, Butyrivibrio or Butyribacterium for butyric acid, the bacterium optionally being genetically modified. The anodic decarboxylation reaction may be performed within the same vessel as the fermentation or separately after fermentation is completed, optionally within a separate electrochemical cell. One or more pairs of anode and cathode electrodes may be used, and the electrodes may be in any shape (e.g., flat, tubular, or corrugated). The anodes are, in various embodiments, made of carbon, graphite, or metal (e.g., gold, platinum, or nickel), while the cathodes are made of platinum, carbon, nickel, iron or alloys. The pH of the electrolysis is generally held between 4.0 and 7.5, preferably between 5.0 and 7.0. In various embodiments, the product is gaseous and is purified via, e.g., distillation.

In one embodiment, low molecular weight (C₂₋₄) olefins are produced by first converting a renewable resource to syngas. Biomass including, but not limited to, corn stover, switchgrass, sugar cane bagasse, sawdust, and a variety of starting materials including, but not limited to, methanol, ethanol, and glycerol can be converted to syngas. Biomass is converted to syngas using a variety of methods, including thermal gasification, thermal pyrolysis and steam reforming, and/or hydrogasification, each of which can produce syngas yields of 70-75% or more. Catalytic steam gasification can give high yields of syngas at relatively low temperatures. The syngas formed is converted via Fischer-Tropsch synthesis using a catalyst with low chain growth probabilities (such as an iron catalyst) to a composition comprising C₂₋₄ olefins, which are then isolated to form a C₂₋₄ olefin-rich stream. Propylene, in various aspects, is isolated from this stream, and ethylene and butylene is subjected to olefin metathesis to produce additional propylene. The propylene, or other olefins, is optionally subjected to a variety of polymerization conditions to yield polypropylene for use in an absorbent article. The process is further described in, e.g., U.S. Patent Application Publication No. US 20100069589.

Olefins alternatively are obtained from triglycerides, optionally obtained from vegetable and/or animal biomass, via hydroconversion and catalytic cracking of a triglycerides feed comprising concentrations of fatty acids above 85%, which maximizes the yields of light olefins while reducing the yield of gasoline. See U.S. Patent Application Publication No. 20090326293. The process comprises, in various aspects, hydroconverting a feed containing triglycerides, in contact with a hydrogen-rich gas stream, on a catalyst of metal oxides to produce three fractions: (1) a fraction of fuel gas and water vapor; (2) a gaseous fraction constituted principally of propane: and (3) a liquid fraction of saturated hydrocarbons (C₉-C₁₈) and dissolved gases. The method further comprises separating the liquid fraction of saturated hydrocarbons; and fluid catalytic cracking the separated liquid fraction in petrochemical conditions with a catalyst bed constituted predominantly of zeolites, in proportions between about 30 and 70 wt. %. The process provides greater selectivity for light olefins, e.g., ethylene and propylene, as well as enhanced conversion when compared with cracking of hydrogenated diesel oil or fluid catalytic cracking of organic oil containing triglycerides, without the hydroconversion stage;

Propylene and other olefins also are obtainable from carboxylic acid, which optionally is generated from sugars and/or other biomass. In one aspect, biomass is fermented to produce carboxylic acid. Other organic intermediates derivable from biomass via fermentation include, but are not limited to, ethanol, butyric acid, 3-hydroxybutyrate, lactic acid, citric acid, succinic acid, malic acid, acetic acid, propionic acid, oxaloacetic acid, and hydroxyalkanoates. The carboxylic acid is then decarboxylated to produce CO² and one or more hydrocarbon compounds, for example, an alkane or an alkene (such as propane or ethylene). Such reactions occur, in various instance, under hydrothermal conditions, and, optionally, without electrolysis of the reactants. For example, if the carboxylic acid (or other organic intermediate) includes a hydroxide moiety, the carboxylic acid is dehydrated, i.e., reacted such that the hydroxide moiety is removed from the molecule as H₂O. A hydrocarbon compound may then be further reacted to produce other compounds, for example, hydrocarbons having at least 4 carbon atoms, and polymers, such as polypropylene.

Decarboxylation of carboxylic acid precursors to yield propylene or precursors thereof is further described in International Patent Publication No. WO 2008/103480, also published as U.S. Patent Application Publication No. 2010228067, which provides reaction conditions for converting polyhydroxybutyrate or butyric acid to propylene at Examples 1 and 2. As described in International Patent Publication No. WO 2008103480, decarboxylation may be performed at, e.g., a temperature of at least about 475 K and/or a pressure of at least about 1.55 MPa. Optionally, a catalyst is used. Suitable catalysts include, but are not limited to, bases (e.g., mineral bases such as KOH or NaOH, or other bases such as dissolved ammonia), oxidizing agents (e.g., hydrogen peroxide), reducing agents (e.g., hydrogen), metal catalysts (e.g., iron, nickel, platinum, palladium, copper), zeolites, acid catalysts (e.g., hydrochloric acid, sulfuric acid, or dissolved carbon dioxide), and metal ion catalysts (e.g., copper ions). Olefins also are obtainable from C₂₋₆ carboxylic acid (e.g., crotonic acid) or a C₂₋₆ carboxylate using, e.g., a decarboxylation-based method. An exemplary decarboxylation-based method is described in International Patent Publication No. WO 2010096812. Carboxylic acids are obtained from biomass by a variety of approaches, such as thermochemical, catalytic, and biochemical treatment. For instance, short-chain aliphatic carboxylic acids or salts thereof are generated from sugars by hydrolysis followed by fermentation. Crotonic acid may be derived from polyhydroxybutyrate using any of the methods described herein. The decarboxylation of the carboxylic acid or carboxylate is carried out using various supported metal catalysts. In various aspects, the method includes contacting a solution containing a C₂₋₆ carboxylic acid or a C₂₋₆ carboxylate with a solid catalyst to form a C₁₋₅ hydrocarbon. If desired, the solution is carried by a gas (e.g., hydrogen, an inert gas, or mixture thereof) to contact the solid catalyst. The solid catalyst includes a metal (e.g., Fe. Co, Ni, Mn, Ru, Rh, Pd, Re, Os, Ir, Pt, Sn, Cu, Ag, and Au) or a combination thereof, and a substrate (e.g., a metal oxide, metal sulfide, metal nitride, metal carbide, a zeolite, a molecular sieve, a perovskite, a clay, and a carbonaceous material) or a combination thereof. A representative catalyst is a 3 wt % Au/Co₃O₄ catalyst. The reaction conditions can vary depending on the solution composition and catalyst used; exemplary reaction conditions include a temperature of about 25° C. to about 500° C. (e.g., about 200° C. to about 350° C. or about 250° C. to about 400° C.) and a pressure of about 1 to about 30 atm (e.g., about 1 to about 10 atm or about 5 to about 15 atm). Exemplary decarboxylation conditions also are described in Bond et al., Langmuir, 26(21), 16291-16298 (2010), and include, e.g., a temperature above 600K at a pressure of 1 bar using an SiO₂/Al₂O₃ catalyst.

Thus, in one aspect of the invention, the element of the absorbent article is generated by converting polyhydroxybutyrate to crotonic acid via thermolysis, and converting the resulting crotonic acid to propylene via decarboxylation. Ethanol is a suitable starting material for producing olefins, including propylene, using, e.g., the methods described in European Patent Application No. 2108635. Ethanol is introduced into a reactor as a stream under a partial pressure of at least about 0.2 MPa. The stream is contacted with a catalyst at conditions effective to convert at least a portion of the ethanol to ethylene, propylene, and olefins having 4 carbon atoms or more (C4+ olefins). The effluent is recovered and fractionated to remove water and unconverted ethanol, thereby producing a stream comprising ethylene and C4+ olefins At least a part of the stream, optionally mixed with a stream comprising C4+ olefins, is introduced into an Olefin Cracking Processing (OCP) reactor. When present, the mixture comprises at least 10% wt. of C4+ olefins. The ethylene stream (optionally comprising the C4+ olefins stream) is reacted with a catalyst selective for light olefins to produce a second effluent with an olefin content of lower molecular weight than that of the feedstock. The second effluent is fractionated to produce at least an ethylene stream, a propylene stream, and a fraction consisting essentially of hydrocarbons having 4 carbon atoms or more. Ethylene is optionally recovered and cracked on a catalyst to give more propylene.

In an alternative process, ethanol is converted to ethylene, olefins having 4 carbon atoms or more (C4+ olefins), and minor amounts of propylene by, e.g., a) introducing in a reactor a stream comprising ethanol under a partial pressure of at least 0.2 MPa and optionally further comprising water and/or inert component; b) contacting the stream with a catalyst under conditions effective to convert at least a portion of the ethanol to ethylene and a C4+ olefin fraction; and c) recovering an effluent comprising ethylene, olefins having 4 carbon atoms or more (C4+ olefins), propylene, and water (optionally also comprising unconverted ethanol and/or inert component). The catalyst is, in various aspects, a crystalline silicate having a ratio Si/Al of at least about 100, a dealuminated crystalline silicate, or a phosphorus modified zeolite. The reaction is carried out at a temperature ranging from about 280° C. to about 500° C. Materials and methods associated with the process are further described in European Patent Application No. 2108635.

Propylene also is derivable from methanol using any of a number of reaction schemes, including chemical processes known in the art. For example, U.S. Pat. No. 7,102,048 describes processes of making a methanol feed and subsequent processing of the methanol feed to produce olefins and/or an olefin stream. In merely a representative process, methanol is first generated from a carbon source (e.g., biomass). One method of generating methanol comprises converting the carbon source to syngas, and converting the syngas to the methanol composition. Conventional processes for converting carbon components to syngas include steam reforming, partial oxidation, and autothermal reforming. For example, contacting a synthesis gas stream with a carbon oxide conversion catalyst forms a crude methanol stream containing methanol, ethanol and acetaldehyde. The methanol composition is separated from the crude methanol stream and contacted with an olefin forming catalyst to form an olefin stream. An example of an olefin forming catalyst is a molecular sieve catalyst, such as, but not limited to, a silicoaluminophosphate molecular sieve.

Another method of forming light olefins, e.g., ethylene and propylene, from methanol and/or from syngas employs a dimethyl ether intermediate. Methanol and/or methanol-containing syngas is exposed to a first catalyst (e.g., an acidic γ-alumina, a modified zeolite, mordenite, a zeolite, ZSM-5, sulfonic acid ion exchange resin, or a perfluorinated sulfonic acid ionomer) to produce dimethyl ether and water. The dimethyl ether is contacted with a second catalyst, such as a molecular sieve catalyst composition (e.g, molecular sieve catalyst composed of SAPO-5, SAPO-8, SAPO-11. SAPO-16, SAPO-17, SAPO-18, SAPO-20, SAPO-31, SAPO-34. SAPO-35, SAPO-36, SAPO-37. SAPO-40, SAPO-41, SAPO-42, SAPO-44, SAPO-47, SAPO-56, ZSM-5, metal containing forms thereof, intergrown forms thereof, AEI/CRA intergrowths, or mixtures thereof) to generate light olefins and water. The first reaction zone (methanol to dimethyl ether) optionally is in a fixed bed reactor, and the second reaction zone (dimethyl ether to light olefin) optionally is in a fluidized reactor. For additional information regarding the materials and methods for converting methanol to propylene via a dimethyl ether intermediate, see, e.g., U.S. Patent Application Publication No. 20060020155.

U.S. Patent Application Publication 20100069691 describes an integrated process for the production of one or more olefins from methanol suitable for use in the context of the invention. In one aspect, the method comprises a fermentation and/or gasification reaction of lignocellulosic materials and/or other organic components contained in residue of a renewable natural agricultural raw material, resulting in the production of a mixture of carbon monoxide and hydrogen (syngas), which is converted to methanol. Propylene is formed from the methanol, directly or indirectly from the intermediate dimethyl ether, using, e.g., the processes described herein and described in U.S. Pat. Nos. 4,929,780 and 6,534,692 and European Patent Application No. 448000. Transformation of methanol and/or dimethyl ether into propylene may take place in one or more reactors arranged in series allowing recycling of unreacted intermediates. Suitable catalysts for propylene formation include, but are not limited to, zeolites of the aluminosilicate, borosilicate and ferrosilicate types; and highly crystalline metallic aluminophosphates comprising, e.g., silicon, magnesium, zinc, iron, cobalt, nickel, manganese, chromium, and mixtures thereof. Any out of reaction conditions promoting formation of polypropylene are used, exemplary conditions being a temperature of from about 250° C. and about 800° C. (e.g., about 300° C. and 550° C.) and a pressure of from about 10 to about 100 kPa, depending on the type of catalyst employed.

Hydrogen activating metal catalyst and high temperatures have been demonstrated to produce hydrocarbon from cellulosic biomass, and are suitable for producing absorbent polymer precursors, such as polypropylene. Japanese Patent Application No. 5213778, for example, describes a high temperature process for generating hydrocarbon from cellulosic biomass using a nickel catalyst. Cellulosic biomass (about 1 pt.wt.), such as wood, is exposed to a hydrogen activating metal catalyst (about 0.1-1 pt.wt.), such as a nickel metal catalyst; an alkali substance (about 0.1-0.5 pt.wt.), such as sodium hydride; and an aqueous medium (about 5-48 pts.wt.) at about 300° C. to about 400° C. under about 90 to about 220 atm H₂.

Glycerol also is a suitable precursor for propylene, as described in International Patent Publication No. WO 2009073938, which discloses a high temperature (about 170° C. to about 380° C.) process for transforming glycerol and/or biomass by heterogenic catalysis for the production of olefins. In some aspects, glycerol used in the catalytic process is raw glycerol from alcoholysis of grease raw materials. Solid acid and/or crystalline basic and/or amorphous catalyzers are described in International Patent Publication No. WO 2009073938 and include, but are not limited to, aluminum oxides, magnesium oxides, silicon oxides, sodium oxides, and calcium oxides. Solid catalysts can be crystalline as zeolites, like MFI, LTA and MOR; and/or lamellar aluminum magnesium oxides, like hydrotalcite; and/or amorphous solids, like Niobia HY(R) and calcium oxide. The crystalline materials are mixed with ligands, inerts, and peptizing and/or chelating agents to obtain catalyzers with desired size, porosity, texture, and catalytic activity. The catalytic process is carried out in fixed or fluidized bed reactors with high temperature, positive pressure varying between about 60 mm Hg to about 11400 mm Hg, and inert atmosphere with nitrogen flux or in the presence of air. In various embodiments, linkage of the reactors to a distillation column for product recovery completes the process.

### III. EXEMPLARY SYNTHETIC POLYMERS

A. Superabsorbent Polymers—Certain compounds derived from renewable resources may be polymerized to yield suitable synthetic superabsorbent polymers. For example, acrylic acid derived from soybean oil via the glycerol/acrolein route described above may be polymerized under the appropriate conditions to yield a superabsorbent polymer comprising poly(acrylic acid). The absorbent polymers useful in the context of the invention can be formed by any polymerization and/or crosslinking techniques capable of achieving the desired properties. Typical methods for producing these polymers are described in Reissue U.S. Pat. No. 32,649 to Brandt et al.; U.S. Pat. Nos. 4,666,983, 4,625,001, 5,408,019; and published German Patent Application No. 4,020,780 to Dahmen. Further processing (i.e., drying, milling, sieving, etc.) of the resulting superabsorbent polymer is well known in the art.

The polymer may be prepared in the neutralized, partially neutralized, or un-neutralized form. In various embodiments, the absorbent polymer may be formed from acrylic acid that is from about 50 mole % to about 95 mole % neutralized. The absorbent polymer may be prepared using a homogeneous solution polymerization process, or by multi-phase polymerization techniques such as inverse emulsion or suspension polymerization procedures. The polymerization reaction will generally occur in the presence of a relatively small amount of di- or poly-functional monomers such as N,N′-methylene bisacrylamide, trimethylolpropane triacrylate, ethylene glycol di(methyacrylate, triallylamine, and methacrylate analogs of the aforementioned acrylates (although this is not required in all aspects of the invention). The di- or poly-functional monomer compounds serve to lightly cross-link the polymer chains, thereby rendering them water-insoluble, yet water-swellable.

In various embodiments, the synthetic superabsorbent polymer comprising acrylic acid derived from renewable resources is formed from starch-acrylic acid aft copolymers, partially neutralized starch-acrylic acid graft copolymers, crosslinked polymers of polyacrylic acid, and crosslinked polymers of partially neutralized polyacrylic acid. Preparation of these materials is disclosed in U.S. Pat. Nos. 3,661,875; 4,076,663; 4,093,776; 4,666,983; and 4,734,478.

Synthetic superabsorbent polymer particles can be surface-crosslinked after polymerization by reaction with a suitable reactive crosslinking agent. Surface-crosslinking of initially formed superabsorbent polymer particles derived from renewable resources provides superabsorbent polymers having relatively high absorbent capacity and relatively high permeability to fluid in the swollen state, as described below. A number of processes for introducing surface crosslinks are disclosed in the art. Suitable methods for surface crosslinking are disclosed in, e.g., U.S. Pat. Nos. 4,541,871, 4,824,901, 4,789,861, 4,587,308, 4,734,478, and 5,164,459; International Patent Publication Nos. WO 199216565, WO 199008789, and WO 199305080; published German Patent Application No. 4,020,780 to Dahmen; and published European Patent Application No. 509,708 to Gartner. Suitable crosslinking agents include di- or poly-functional crosslinking reagents such as di/poly-haloalkanes, di/poly-epoxides, di/poly-acid chlorides, di/poly-tosyl alkanes, di/poly-aldehydes, di/poly-alcohols, and the like.

An important characteristic of the synthetic superabsorbent polymers of the invention is the permeability or flow conductivity of a zone or layer of the polymer particles when swollen with body fluids. This permeability or flow conductivity is defined herein in terms of the Saline Flow Conductivity (SFC) value of the superabsorbent polymer. SFC measures the ability of the swollen hydrogel zone or layer to transport or distribute body fluids under usage pressures. It is believed that when a superabsorbent polymer is present at high concentrations in an absorbent member and then swells to form a hydrogel under usage pressures, the boundaries of the hydrogel come into contact, and interstitial voids in this high-concentration region become generally bounded by hydrogel. When this occurs, it is believed the permeability or flow conductivity properties of this region are generally reflective of the permeability or flow conductivity properties of a hydrogel zone or layer formed from the superabsorbent polymer alone. It is further believed that increasing the permeability of these swollen high-concentration regions to levels that approach or even exceed conventional acquisition/distribution materials, such as wood-pulp fluff, can provide superior fluid handling properties for the absorbent member and absorbent core, thus decreasing incidents of leakage, especially at high fluid loadings. Higher SFC values also are reflective of the ability of the formed hydrogel to acquire body fluids under normal usage conditions.

The SFC value of the synthetic superabsorbent polymers derived from renewable resources useful in the invention is at least about 30×10⁻⁷ cm³ sec/g. In other embodiments, the SFC value of the superabsorbent polymers is at least about 50×10⁻⁷ cm ³ sec/g. In other embodiments, the SFC value of the superabsorbent polymers is at least about 100×10⁻⁷ cm³ sec/g. Typically, these SFC values are in the range of from about 30×10⁻⁷ to about 1000×10⁻⁷ cm³ sec/g. However, SFC values ay range from about 50×10⁻⁷ to about 500×10⁻⁷ cm³ sec/g or from about 50×10⁻⁷ to about 350×10⁻⁷ cm³ sec/g. A method for determining the SFC value of the superabsorbent polymers is provided hereafter in the Test Methods Section.

Another important characteristic of the superabsorbent polymers of the invention is their ability to swell against a load. This capacity versus a load is defined in terms of the superabsorbent polymer's Absorption Against Pressure (AAP) capacity. When a superabsorbent polymer is incorporated into an absorbent member at high concentrations, the polymer needs to be capable of absorbing large quantities of body fluids in a reasonable time period under usage pressures. Usage pressures exerted on the superabsorbent polymers used within absorbent article include both mechanical pressures (e.g., exerted by the weight and motions of a wearer, taping forces, etc.) and capillary pressures (e.g., resulting from the acquisition component(s) in the absorbent core that temporarily hold fluid before it is absorbed by the superabsorbent polymer).

The AAP capacity of absorbent polymer is generally at least about 15 g/g. In certain embodiments, the AAP capacity of absorbent polymer is generally at least about 20 g/g. Typically, AAP values range from about 15 to about 25 g/g. However, AAP values may range from about 17 to about 23 g/g or from about 20 to about 23 g/g. A method for determining the AAP capacity value of these absorbent polymers is provided hereafter in the Test Methods Section.

B. Polyolefins—Olefins derived from renewable resources may be polymerized to yield polyolefins. Ethylene derived from renewable resources may be polymerized under appropriate conditions to prepare polyethylene having desired characteristics for use in a particular component of an absorbent article or in the packaging for the article. The polyethylene may be high density, medium density, low density, or linear-low density. Polyethylene and/or polypropylene may be produced via free-radical polymerization techniques, or by using Ziegler-Natta catalysis or Metallocene catalysts.

The polyolefin is processed according to any suitable method, such as methods known in the art, into a form suitable for the end use of the polymer. Suitable forms for polyolefins include a film, an apertured film, a microporous film, a fiber, a filament, a nonwoven, or a laminate. Suitable nonwoven forms include spunbond webs, meltblown webs, and combinations thereof (e.g., spunbond-meltblown webs (SM), spunbond-meltblown-spunbond webs (SMS), etc.). The polyolefin may comprise mixtures or blends with other polymers such as polyolefins derived from petrochemicals. Depending on the end use and form, the polyolefin optionally comprises other compounds such as inorganic compounds. fillers, pigments, dyes, antioxidants. UV-stabilizers, binders, surfactants, wetting agents, and the like. For example, a polyolefin film may be impregnated with inorganic compound such as calcium carbonate, titanium dioxide, clays, silicas, zeolites, kaolin, mica, carbon, and mixtures thereof. Such compounds may serve as pore forming agents which, upon straining the film, improve the breathability of the film. This process is described further in U.S. Pat. No. 6,605,172. A binder may be used with a polyolefin fibers, filaments, or nonwoven web. A suitable binder is a styrene-butadiene latex binder available under the trade name GENFLO™ 3160 from OMNOVA Solutions Inc.; Akron, Ohio. The resulting binder/polyolefin web may be used as an acquisition layer, which may be associated with the absorbent core. The polyolefin materials and particularly polyolefin fibers, filaments, and nonwoven webs may treated with a surfactant or wetting agent such as Irgasurf™ available from Ciba Specialty Chemicals of Tarrytown, N.Y.

Polyolefin nonwovens useful in an absorbent article may have a basis weight between about 1 g/m² and about 50 g/m² or between about 5 g/m² and about 30 g/m², as measured according to the Basis Weight Test provided below. Polyolefin nonwovens suitable for use as a topsheet may have an average liquid strike-through time of less than about 4 seconds, as measured according to the Liquid Strike-Through Test provided below. In other embodiments the polyolefin nonwoven may have an average strike-through time of less than about 3 seconds or less than about 2 seconds.

Polyolefin nonwoven useful as a barrier leg cuff may have a hydrohead of greater than about 5 mbar or about 6 mbar and less than about 10 mbar or about 8 mbar, as measured according to the Hydrohead test provided below,

Polyolefin films suitable for use as a backsheet may have an MD tensile strength of greater than about 0.5 N/cm or about 1 N/cm and less than about 6 N/cm or about 5 N/cm. as measured according to the Tensile Test as provided below. For breathable polyolefin films suitable for use as a backsheet, the film may have a Moisture Vapor Transmission Rate (MVTR) of at least about 2000 g/m²/hr, preferably greater than about 2400 g/m²/hr, and even more preferably greater than about 3000 g/m²/hr, as measured by the Moisture Vapor Transmission Rate test provided below. It should be recognized that non-breathable backsheets, which are also useful in diapers, would exhibit an MVTR value of about 0 g/m²/hr.

C. Other Polymers—It should be recognized that any of the aforementioned synthetic polymers may be formed by using a combination of monomers derived from renewable resources and monomers derived from non-renewable (e.g., petroleum) resources. For example, the superabsorbent polymer of poly(acrylic acid) may be polymerized from a combination of acrylic acid derived from renewable resources and acrylic acid derived from non-renewable resources. The monomer derived from a renewable resource may comprise at least about 5% by weight [weight of renewable resource monomer/weight of resulting polymer×100], at least about 10% by weight, at least about 20% by weight, at least about 30% by weight, at least about 40% by weight, or at least about 50% by weight of the superabsorbent polymer.

### IV. ABSORBENT ARTICLES COMPRISING THE SYNTHETIC POLYMER DERIVED FROM RENEWABLE RESOURCES

The invention relates to an absorbent article comprising a synthetic polymer derived from a renewable resource. The polymer has specific performance characteristics. The polymers derived from a renewable resource may be in any suitable form such as a film, nonwoven, superabsorbent, and the like.

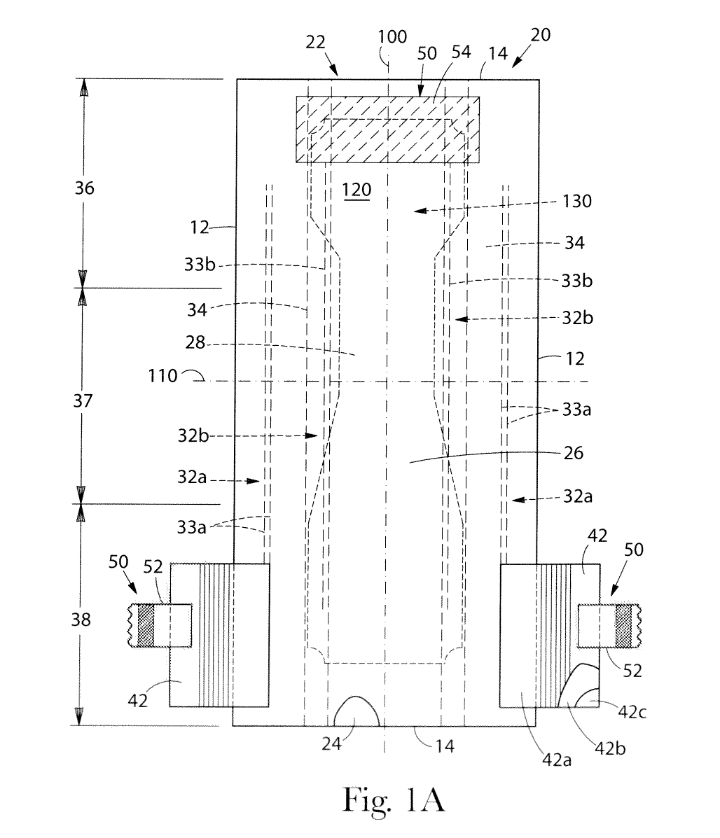

FIG. 1A is a plan view of an exemplary, non-limiting embodiment of an absorbent article in the form of a diaper 20 in a flat, uncontracted state (i.e., without elastic induced contraction). The garment-facing surface 120 of the diaper 20 is facing the viewer and the body-facing surface 130 is opposite the viewer. The diaper 20 includes a longitudinal centerline 100 and a lateral centerline 110. FIG. 1B is a cross-sectional view of the diaper 20 of FIG. 1A taken along the lateral centerline 110. The diaper 20 may comprise a chassis 22. The diaper 20 and chassis 22 are shown to have a front waist region 36, a rear waist region 38 opposed to the front waist region 36, and a crotch region 37 located between the front waist region 36 and the rear waist region 38. The waist regions 36 and 38 generally comprise those portions of the diaper 20 which, when worn, encircle the waist of the wearer. The waist regions 36 and 38 may include elastic elements such that they gather about the waist of the wearer to provide improved fit and containment. The crotch region 37 is that portion of the diaper 20 which, when the diaper 20 is worn, is generally positioned between the legs of the wearer.

The outer periphery of diaper 20 and/or chassis 22 is defined by longitudinal edges 12 and lateral edges 14. The chassis 22 may have opposing longitudinal edges 12 that are oriented generally parallel to the longitudinal centerline 100. However, for better fit, longitudinal edges 12 may be curved or angled to produce, for example, an “hourglass” shape diaper when viewed in a plan view. The chassis 22 may have opposing lateral edges 14 that are oriented generally parallel to the lateral centerline 110.

The chassis 22 may comprises a liquid permeable topsheet 24, a backsheet 26, and an absorbent core 28 between the topsheet 24 and the backsheet 26. The absorbent core 28 may have a body-facing surface and a garment facing-surface. The topsheet 24 may be joined to the core 28 and/or the backsheet 26. The backsheet 26 may be joined to the core 28 and/or the topsheet 24. It should be recognized that other structures, elements, or substrates may be positioned between the core 28 and the topsheet 24 and/or backsheet 26. In certain embodiments, the chassis 22 comprises the main structure of the diaper 20 and other features may added to form the composite diaper structure. The topsheet 24, the backsheet 26, and the absorbent core 28 may be assembled in a variety of well-known configurations as described generally in U.S. Pat. Nos. 3,860,003; 5,151,092; 5,221,274; 5,554,145; 5,569,234; 5,580,411; and 6,004,306.

The absorbent core 28 may comprise the superabsorbent polymer derived from a renewable resource of the invention as well as a wide variety of other liquid-absorbent materials commonly used in diapers and other absorbent articles. Examples of suitable absorbent materials include comminuted wood pulp, which is generally referred to as air felt; chemically stiffened, modified or cross-linked cellulosic fibers; superabsorbent polymers or absorbent gelling materials; melt blown polymers, including co-form, biosoluble vitreous microfibers; tissue, including tissue wraps and tissue laminates; absorbent foams; absorbent sponges; and any other known absorbent material or combinations of materials. Exemplary absorbent structures for use as the absorbent core 28 are described in U.S. Pat. Nos. 4,610,678; 4,673,402; 4,834,735; 4,888,231; 5,137,537: 5,147,345; 5,342,338; 5,260,345; 5,387,207; 5,397,316; 5,625,222; and 6,932,800. Further exemplary absorbent structures may include non-removable absorbent core components and removable absorbent core components. Such structures are described in U.S. Patent Application Publications 20040039361; 20040024379; 20040030314; 20030199844; and 20050228356. Ideally, the absorbent core 28 may be comprised entirely of materials derived from renewable resources: however, the absorbent core 28 may comprise materials derived from non-renewable resources.

The absorbent core 28 may comprise a fluid acquisition component, a fluid distribution component, and a fluid storage component. A suitable absorbent core 28 comprising an acquisition layer, a distribution layer, and a storage layer is described in U.S. Pat. No. 6,590,136.

Another suitable absorbent core construction where the superabsorbent polymer of the invention may be used is described in U.S. Patent Application Publication No. 20040167486 to Busam et al. The absorbent core of the aforementioned publication uses no or, in the alternative, minimal amounts of absorbent fibrous material within the core. Generally, the absorbent core may include no more than about 20% weight percent of absorbent fibrous material (i.e., [weight of fibrous material/total weight of the absorbent core]×100).

The topsheet 24 is generally a portion of the diaper 20 that may be positioned at least in partial contact or close proximity to a wearer. Suitable topsheets 24 may be manufactured from a wide range of materials such as woven or nonwoven webs of natural fibers (e.g., wood or cotton fibers), synthetic fibers (e.g., polyester or polypropylene fibers), or a combination of natural and synthetic fibers; apertured plastic films; porous foams or reticulated foams. The topsheet 24 is generally supple, soft feeling, and non-irritating to a wearer's skin. Generally, at least a portion of the topsheet 24 is liquid pervious, permitting liquid to readily penetrate through the thickness of the topsheet 24. Suitably, the topsheet 24 comprises a polymer (e.g. polyethylene) derived from a renewable resource. Alternately, a suitable topsheet 24 is available from BBA Fiberweb, Brentwood, Tenn. as supplier code 055SLPV09U.

Any portion of the topsheet 24 may be coated with a lotion as is known in the art. Examples of suitable lotions include those described in U.S. Pat. Nos. 5,607,760; 5,609,587; 5,635,191; and 5,643,588. The topsheet 24 may be fully or partially elasticized or may be foreshortened so as to provide a void space between the topsheet 24 and the core 28. Exemplary structures including elasticized or foreshortened topsheets are described in more detail in U.S. Patent Nos. 4,892,536; 4,990,147; 5,037,416; and 5.269,775.

The backsheet 26 is generally positioned such that it may be at least a portion of the garment-facing surface 120 of the diaper 20. Backsheet 26 may be designed to prevent the exudates absorbed by and contained within the diaper 20 from soiling articles that may contact the diaper 20, such as bed sheets and undergarments. In certain embodiments, the backsheet 26 is substantially water-impermeable; however, the backsheet 26 may be made breathable so as to permit vapors to escape while preventing liquid exudates from escaping. The polyethylene film may be made breathable by inclusion of inorganic particulate material and subsequent tensioning of the film. Breathable backsheets may include materials such as woven webs, nonwoven webs, composite materials such as film-coated nonwoven webs, and microporous films. Suitably, the backsheet 26 comprises a polymer such (e.g. polyethylene) derived from a renewable resource as disclosed above. Alternative backsheets 26 derived from non-renewable resources include films manufactured by Tredegar Industries Inc. of Terre Haute, Ind. and sold under the trade names X15306. X10962, and X10964; and microporous films such as manufactured by Mitsui Toatsu Co., of Japan under the designation ESPOIR NO and by EXXON Chemical Co., of Bay City, Tex., under the designation EXXAIRE. Other alternative breathable backsheets 26 are described in U.S. Pat. Nos. 5,865,823, 5,571,096, and 6,107,537.

Backsheet 26 may also consist of more than one layer. For example, the backsheet 26 may comprise an outer cover and an inner layer or may comprise two outer layers with an inner layer disposed therebetween. The outer cover may have longitudinal edges and the inner layer may have longitudinal edges. The outer cover may be made of a soft, non-woven material. The inner layer may be made of a substantially water-impermeable film. The outer cover and an inner layer may be joined together by adhesive or any other suitable material or method. Suitably, the nonwoven outer cover and the water-impermeable film comprise polymers (e.g., polyethylene) may be derived from renewable resources. Alternatively, a suitable outer cover and inner layer derived from non-renewable resources are available, respectively, as supplier code A18AH0 from Corovin GmbH, Peine, Germany and as supplier code PGBR4WPR from RKW Gronau GmbH, Gronau, Germany. While a variety of backsheet configurations are contemplated herein, it would be obvious to those skilled in the art that various other changes and modifications can be made without departing from the spirit and scope of the invention.

The diaper 20 may include a fastening system 50. When fastened, the fastening system 50 interconnects the front waist region 36 and the rear waist region 38. When fastened, the diaper 20 contains a circumscribing waist opening and two circumscribing leg openings. The fastening system 50 may comprise an engaging member 52 and a receiving member 54. The engaging member 52 may comprise hooks, loops, an adhesive, a cohesive, a tab, or other fastening mechanism. The receiving member 54 may comprise hooks, loops, a slot, an adhesive, a cohesive, or other fastening mechanism that can receive the engaging member 52. Suitable engaging member 52 and receiving member 54 combinations are well known in the art and include but are not limited to hooks/loop, hooks/hooks, adhesive/polymeric film, cohesive/cohesive, adhesive/adhesive, tab/slot, and button/button hole. Suitably, the fastening system 50 may comprise a polymer (e.g., polyethylene film or a polyethylene nonwoven) derived from a renewable resource.

The diaper 20 may include front ears (not shown) and/or back ears 42. The front and/or back ears 42 may be unitary elements of the diaper 20 (i.e., they are not separately manipulative elements secured to the diaper 20, but rather are formed from and are extensions of one or more of the various layers of the diaper). In certain embodiments, the front and/or back ears 42 may be discrete elements that are joined to the chassis 22, as shown in FIG. 1A. Discrete front and/or back ears 42 may be joined to the chassis 22 by any bonding method known in the art such as adhesive bonding, pressure bonding, heat bonding, and the like. In other embodiments, the front and/or back ears 42 may comprise a discrete element joined to the chassis 22 with the chassis 22 having a layer, element, or substrate that extends over the front and/or back ear 42. The front ears and back ears 42 may be extensible, inextensible, elastic, or inelastic. The front ears and back ears 42 may be formed from nonwoven webs, woven webs, knitted fabrics, polymeric and elastomeric films, apertured films, sponges, foams, scrims, and combinations and laminates thereof. In certain embodiments the front ears and back ears 42 may be formed of a stretch laminate comprising a first nonwoven 42a, elastomeric material 42b, and, optionally, a second nonwoven 42c or other like laminates. The first and second nonwoven 42a, 42c may comprise a synthetic polymer (e.g., polyethylene) derived from a renewable resource. A suitable elastomeric material 42b may comprise a natural elastomer such as natural rubber or may comprise a synthetic elastomer such as the elastomeric film available from Tredegar Corp, Richmond, Va., as supplier code X25007. An alternate stretch laminate may be formed from the Tredegar X25007 elastomer disposed between two nonwoven layers (available from BBA Fiberweb, Brentwood, Tenn. as supplier code FPN332).

The diaper 20 may further include leg cuffs 32a, 32b which provide improved containment of liquids and other body exudates. Leg cuffs 32a, 32b may also be referred to as gasketing cuffs, outer leg cuffs, leg bands, side flaps, elastic cuffs, barrier cuffs, second cuffs, inner leg cuffs, or “stand-up” elasticized flaps. U.S. Pat. No. 3,860,003 describes a disposable diaper which provides a contractible leg opening having a side flap and one or more elastic members to provide an elasticized leg cuff (i.e., a gasketing cuff). U.S. Pat. Nos. 4,808,178 and 4,909,803 describe disposable diapers having “stand-up” elasticized flaps (i.e., barrier cuffs) which improve the containment of the leg regions. U.S. Pat. Nos. 4,695,278 and 4,795,454 describe disposable diapers having dual cuffs, including gasketing cuffs and barrier cuffs.

FIGS. 1A-B shows the diaper 20 having dual cuffs: gasketing cuff 32a and barrier cuff 32b. The barrier cuff 32b may include one or more barrier elastic members 33b. The barrier elastic members 33b may be joined to a barrier cuff substrate 34. The barrier cuff substrate 34 may comprise a polymer derived from a renewable resource. In certain embodiments, the barrier cuff substrate 34 may be a polymeric film or nonwoven. The barrier cuff 32b may be disposed on the body-facing surface of the chassis 22. The barrier cuff substrate 34 may extend laterally from the longitudinal edge 12 of the chassis 22 to a point inboard of the longitudinal edge 122. The barrier cuff 32b generally extends longitudinally at least through the crotch region 37. The barrier elastic members 33b allow a portion of the barrier cuff 32b to be spaced away from the body-facing surface of the diaper 20.

The gasketing cuff 32a may include one or more gasketing elastic members 33a. The gasketing elastic member 33a may be joined to one or more of the existing elements or substrates of the diaper 20 (e.g., topsheet 24, backsheet 26, barrier cuff substrate 34, etc.). In some embodiments, it may be desirable to treat all or a portion of the leg cuffs 32 with a hydrophilic surface coasting such as is described in U.S. Patent Application Publication No. 20050177123A1. Suitable gasketing and barrier elastic members 33a, 33b include natural rubber, synthetic rubbers, and other elastomers.

In other suitable embodiments, the diaper 20 may be preformed by the manufacturer to create a pant. A pant may be preformed by any suitable technique including, but not limited to, joining together portions of the article using refastenable and/or non-refastenable bonds (e.g., seam, weld, adhesive, cohesive bond, fastener, etc.). For example, the diaper 20 of FIG. 1A may be manufactured with the fastening system 50 engaged (i.e., the engaging member 52 is joined to the receiving member 54). As an additional example, the diaper 20 of FIG. 1A may be manufactured with the front ears 40 joined to the back ears 42 by way of a bond such as an adhesive bond, a mechanical bond, or some other bonding technique known in the art. Suitable pants are disclosed in U.S. Pat. Nos. 5,246,433; 5,569,234; 6,120,487; 6,120,489; 4,940,464; 5,092,861; 5,897,545; and 5,957,908.

### V. PROVIDING THE ABSORBENT ARTICLE TO A CONSUMER

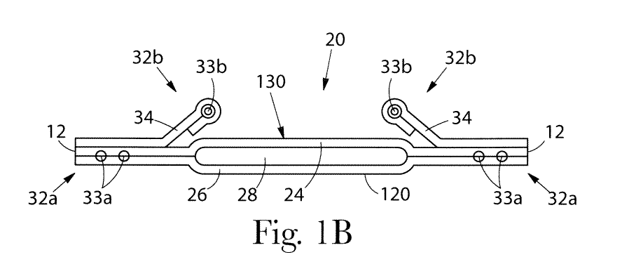

One or more absorbent articles (e.g, diapers) 220 may be provided as a package 200, as shown in FIGS. 2A-B. Generally, the package 200 allows for a quantity of absorbent articles 220 to be delivered to and purchased by a consumer while economizing space and simplifying transport and storage. The package 200 includes at least one absorbent article 220 secured by an overwrap 250. The overwrap 250 may partially or fully cover the absorbent article(s), which may be compressed or uncompressed. FIG, 2A depicts an overwrap 250 that completely covers and encases a plurality of absorbent articles 220. The overwrap 250 may comprise a variety of materials including, but not limited to, thermoplastic films, nonwovens, wovens, foils, fabrics, papers, cardboard, elastics, cords, straps, and combinations thereof. Other suitable package structures and overwraps are described in U.S. Pat. Nos. 4,846,587; 4,934,535; 4,966,286; 5,036,978; 5,050,742; and 5,054,619. In certain embodiments, the overwrap 250 comprises a synthetic polymer (e.g., a polyolefin) derived from a renewable resource. While the package 200 is not limited in shape, it may be desirable for the package 200 to have the shape of a parallelepiped or substantially similar to a parallelepiped (e.g., a solid at least a substantially planar base and four substantially planar sides). Such a shape is ideal for packaging, stacking, and transport. The package 200 is not limited in size; however, in certain embodiments, the size of the package 200 should be no greater than is required to contain the absorbent articles 220. The package 200 may have a handle 240. In certain embodiments, the handle 240 may be a discrete element such as a strap that may be affixed to the overwrap 250. In the embodiment shown in FIGS. 2A-B, the handle 240 is integral to the overwrap 250. For this embodiment, the handle 240 may comprise an extension 252 from the overwrap 250. The extension 252 may have an aperture 254 there through. The aperture 254 ideally sized to permit entry by one or more digits of an adult hand.

An opening device 260 may be provided in the overwrap 250. For example, the opening device 260 may comprise a line of weakness 262 (e.g., perforations) in an overwrap 250 made from paper, cardboard, or film. The opening device 260 allows for partial or full removal of a flap 256 which is a portion of the overwrap 250. Partial of full removal of the flap 256 may allow for improved access to the absorbent articles 220. The opening device 260 and flap 256 are shown in a closed configuration in FIG. 2A and in an open configuration in FIG. 2B. An exemplary opening device 260 is presented in U.S. Pat. No. 5,036,978.

The package 200 may contain multiple overwraps 250. For example, a plurality of absorbent articles may be secured with a first overwrap such as a thermoplastic film and then a plurality of film wrapped absorbent articles may be secured in a second overwrap such as a cardboard box or another thermoplastic film.

### VI. COMMUNICATING A RELATED ENVIRONMENTAL MESSAGE A CONSUMER

The invention may further comprise a related environmental message or may further comprise a step of communicating a related environmental message to a consumer. The related environmental message may convey the benefits or advantages of the absorbent article comprising a polymer derived from a renewable resource. The related environmental message may identify the absorbent articles as: being environmentally friendly or Earth friendly; having reduced petroleum (or oil) dependence or content: having reduced foreign petroleum (or oil) dependence or content; having reduced petrochemicals or having components that are petrochemical free; and/or being made from renewable resources or having components made from renewable resources. This communication is of importance to consumers that may have an aversion to petrochemical use (e.g., consumers concerned about depletion of natural resources or consumers who find petrochemical based products unnatural or not environmentally friendly) and to consumers that are environmentally conscious. Without such a communication, the benefit of the invention may be lost on some consumers.

The communication may be effected in a variety of communication forms. Suitable communication forms include store displays, posters, billboard, computer programs, brochures, package literature, shelf information, videos, advertisements, internet web sites, pictograms, iconography, or any other suitable form of communication. The information could be available at stores, on television, in a computer-accessible form, in advertisements, or any other appropriate venue. Ideally, multiple communication forms may be employed to disseminate the related environmental message.

The communication may be written, spoken, or delivered by way of one or more pictures, graphics, or icons. For example, a television or internet based-advertisement may have narration, a voice-over, or other audible conveyance of the related environmental message. Likewise, the related environmental message may be conveyed in a written form using any of the suitable communication forms listed above. In certain embodiments, it may be desirable to quantify the reduction of petrochemical usage of the present absorbent article compared to absorbent articles that are presently commercially available.

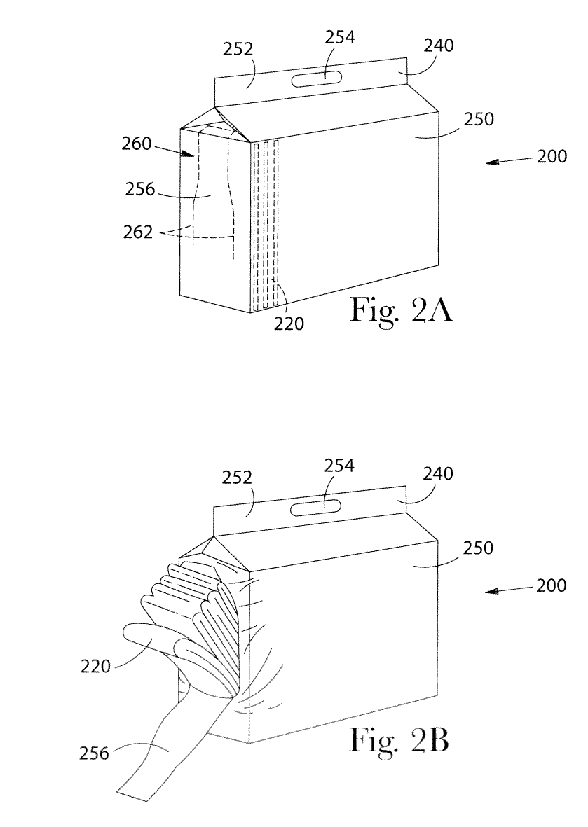

In other embodiments, the communication form may be one or more icons. FIGS. 3A-F depict several suitable embodiments of a communication in the form of icon 310. One or more icons 310 may be used to convey the related environmental message of reduced petrochemical usage. Suitable icons 310 communicating the related environmental message of reduced petroleum usage are shown in FIGS. 3A-B. Icons communicating the related environmental message of environmental friendliness or renewable resource usage are shown in FIGS. 3C-F. In certain embodiments, the icons 310 may be located on the package 200 (as shown in FIGS. 2A-B) containing the absorbent articles, on the absorbent article, on an insert adjoining the package or the articles, or in combination with any of the other forms of the communication listed above.

The related environmental message may also include a message of petrochemical equivalence. As presented in the Background, many renewable, naturally occurring, or non-petroleum derived polymers have been disclosed. However, these polymers often lack the performance characteristics that consumers have come to expect when used in absorbent articles. Therefore, a message of petroleum equivalence may be necessary to educate consumers that the polymers derived from renewable resources, as described above, exhibit equivalent or better performance characteristics as compared to petroleum derived polymers. A suitable petrochemical equivalence message can include comparison to an absorbent article that does not have a polymer derived from a renewable resource. For example, a suitable combined message may be, “Diaper Brand A with an environmentally friendly absorbent material is just as absorbent as Diaper Brand B.” This message conveys both the related environmental message and the message of petrochemical equivalence.

### VII. METHOD OF MAKING AN ABSORBENT ARTICLE HAVING A POLYMER DERIVED FROM A RENEWABLE RESOURCE

The invention further relates to a method for making an absorbent article comprising a superabsorbent polymer derived from a renewable resource. The method comprises the steps of providing a renewable resource; deriving a monomer from the renewable resource (optionally using any one or more of the methods described herein); polymerizing the monomer. In various embodiments, polymerization forms a synthetic superabsorbent polymer having a Saline Flow Conductivity value of at least about 30×10⁻⁷ cm³·sec/g and an Absorption Against Pressure value of at least about 15 g/g; and incorporating said superabsorbent polymer into an absorbent article. The invention further relates to providing one or more of the absorbent articles to a consumer and communicating reduced petrochemical usage to the consumer. The polymer derived from renewable resources may undergo additional process steps prior to incorporation into the absorbent article. Such process steps include drying, sieving, surface crosslinking, and the like.

The invention further relates to a method for making an absorbent article comprising a synthetic polyolefin derived from a renewable resource. The method comprises the steps of providing a renewable resource; deriving an olefin monomer from the renewable resource; polymerizing the monomer to form a synthetic polyolefin having a ¹⁴C/C ratio of about 1.0×10⁻¹⁴ or greater; and incorporating said polyolefin into an absorbent article. The synthetic polyolefin exhibits one or more of the above referenced performance characteristics. The invention further relates to providing one or more of the absorbent articles to a consumer and communicating reduced petrochemical usage to the consumer. The polymer derived from renewable resources may undergo additional process steps prior to incorporation into the absorbent article. Such process steps include, film formation, fiber formation, ring rolling, and the like.

### VIII. VALIDATION OF POLYMERS DERIVED FROM RENEWABLE RESOURCES

A suitable validation technique is through ¹⁴C analysis. A common analysis technique in carbon-14 dating is measuring the ratio of ¹⁴C to total carbon within a sample (¹⁴C/C). Research has noted that fossil fuels and petrochemicals generally have a ¹⁴C/C ratio of less than about 1×10⁻¹⁵. However, polymers derived entirely from renewable resources typically have a ¹⁴C/C ratio of about 1.2×10⁻¹². When compared, the polymers derived from renewable resources may have a ¹⁴C/C ratio three orders of magnitude (10³=1,000) greater than the ¹⁴C/C ratio of polymers derived from petrochemicals. Polymers useful in the invention have a ¹⁴C/C ratio of about 1.0×10⁻¹⁴ or greater. In other embodiments, the petrochemical equivalent polymers of the invention may have a ¹⁴C/C ratio of about 1.0×10⁻¹³ or greater or a ¹⁴C/C ratio of about 1.0×10⁻¹² or greater. Suitable techniques for ¹⁴C analysis are known in the art and include accelerator mass spectrometry, liquid scintillation counting, and isotope mass spectrometry. These techniques are described in U.S. Pat. Nos. 3,885,155; 4,427,884; 4,973,841; 5,438,194; and 5,661,299.

### IX. ASSESSMENT OF THE BIO-BASED CONTENT OF MATERIALS

One or more elements of the absorbent article (e.g., the absorbent core, topsheet, dusting layer, backsheet, barrier leg cuff, and/or fastening system) comprise at least about 50% (e.g., at least about 60%, at least about 65%, at least about 70%, at least about 75%, at least about 80%, at least about 85%, at least about 90%, or at least about 95%) bio-based content based on the total weight of the element. In this regard, the synthetic polymer is composed of a sufficient amount of bio-based components (i.e., the precursors are substantially composed of materials derived from renewable resources), and the element comprises a sufficient amount of the synthetic polymer bio-acrylate), to achieve the desired bio-based content level. In various embodiments, the element comprises a small percentage of petroleum based material and/or post-industrial recycled polymer and/or post-consumer recycled polymer.

A suitable method to assess materials derived from renewable resources is through ASTM D6866, which allows the determination of the bio-based content of materials using radiocarbon analysis by accelerator mass spectrometry, liquid scintillation counting, and isotope mass spectrometry. When nitrogen in the atmosphere is struck by an ultraviolet light produced neutron, it loses a proton and forms carbon that has a molecular weight of 14, which is radioactive. This ¹⁴C is immediately oxidized into carbon dioxide, which represents a small, but measurable fraction of atmospheric carbon. Atmospheric carbon dioxide is cycled by green plants to make organic molecules during the process known as photosynthesis. The cycle is completed when the green plants or other forms of life metabolize the organic molecules producing carbon dioxide, which causes the release of carbon dioxide back to the atmosphere. Virtually all forms of life on Earth depend on this green plant production of organic molecules to produce the chemical energy that facilitates growth and reproduction. Therefore, the ¹⁴C that exists in the atmosphere becomes part of all life forms and their biological products. These renewably based organic molecules that biodegrade to carbon dioxide do not contribute to global warming because no net increase of carbon is emitted to the atmosphere. In contrast, fossil fuel-based carbon does not have the signature radiocarbon ratio of atmospheric carbon dioxide. See International Patent Publication No. WO 2009155086, incorporated herein by reference.

The application of ASTM D6866 to derive a “bio-based content” is built on the same concepts as radiocarbon dating, but without use of the age equations. The analysis is performed by deriving a ratio of the amount of radiocarbon (¹⁴C) in an unknown sample to that of a modern reference standard. The ratio is reported as a percentage with the units “pMC” (percent modern carbon). If the material being analyzed is a mixture of present day radiocarbon and fossil carbon (containing no radiocarbon), then the pMC value obtained correlates directly to the amount of biomass material present in the sample.

The modern reference standard used in radiocarbon dating is a NIST (National Institute of Standards and Technology) standard with a known radiocarbon content equivalent approximately to the year AD 1950. The year AD 1950 was chosen because it represented a time prior to thermo-nuclear weapons testing, which introduced large amounts of excess radiocarbon into the atmosphere with each explosion (termed “bomb carbon”). The AD 1950 reference represents 100 pMC.

“Bomb carbon” in the atmosphere reached almost twice normal levels in 1963 at the peak of testing and prior to the treaty halting the testing. Its distribution within the atmosphere has been approximated since its appearance, showing values that are greater than 100 pMC for plants and animals living since AD 1950. The distribution of bomb carbon has gradually decreased over time, with today's value being near 107.5 pMC. As a result, a fresh biomass material, such as corn, could result in a radiocarbon signature near 107.5 pMC.

Petroleum-based carbon does not have the signature radiocarbon ratio of atmospheric carbon dioxide. Research has noted that fossil fuels and petrochemicals have less than about 1 pMC, and typically less than about 0.1 pMC, for example, less than about 0.03 pMC. However, compounds derived entirely from renewable resources have at least about 95 percent modern carbon (pMC), preferably at least about 99 pMC, for example, about 100 pMC.

Combining fossil carbon with present day carbon into a material will result in a dilution of the present day pMC content. By presuming that 107.5 pMC represents present day biomass materials and 0 pMC represents petroleum derivatives, the measured pMC value for that material will reflect the proportions of the two component types. A material derived 100% from present day soybeans would give a radiocarbon signature near 107.5 pMC. If that material was diluted with 50% petroleum derivatives, it would give a radiocarbon signature near 54 pMC.

A bio-based content result is derived by assigning 100% equal to 107.5 pMC and 0% equal to 0 pMC. In this regard, a sample measuring 99 pMC will give an equivalent bio-based content result of 93%.

Assessment of the materials described herein may be accomplished with ASTM D6866, particularly with Method B. It is generally presumed that all materials are present day or fossil in origin and that the desired result is the amount of bio-component “present” in the material, not the amount of bio-material “used” in the manufacturing process. Other techniques for assessing the bio-based content of materials are described in U.S. Pat. Nos. 3,885,155; 4,427,884; 4,973,841; 5,438,194; and 5,661,299; and International Patent Publication No. WO 2009155086, each of which is incorporated herein by reference.

### X. TEST METHODS

### Saline Flow Conductivity

The method to determine the permeability of a swollen hydrogel layer 718 is the “Saline Flow Conductivity” also known as “Gel Layer Permeability” and is described in several references, including, EP A 640 330, filed on Dec. 1, 1993, U.S. Ser. No. 11/349,696, filed on Feb. 3, 2004, U.S. Ser. No. 11/347,406, filed on Feb. 3, 2006, U.S. Ser. No. 06/682,483, filed on Sep. 30, 1982, and U.S. Pat. No. 4,469,710, filed on Oct. 14, 1982. The equipment used for this method is described below.

Permeability Measurement System

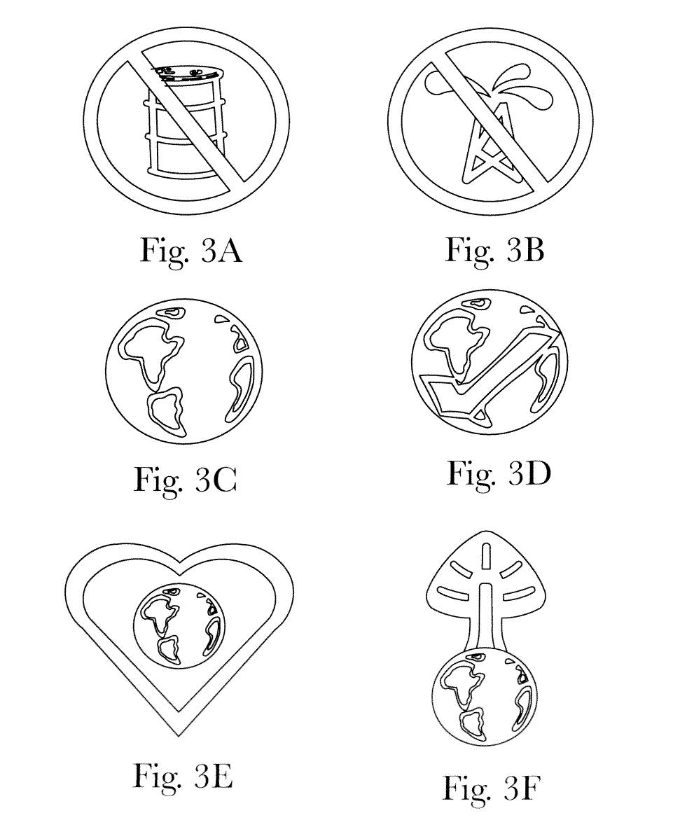

FIG. 4 shows permeability measurement system 400 set-up with the constant hydrostatic head reservoir 414, open-ended tube for air admittance 410, stoppered vent for refilling 412, laboratory jack 416, delivery tube 418, stopcock 420, ring stand support 422, receiving vessel 424, balance 426 and piston/cylinder assembly 428.

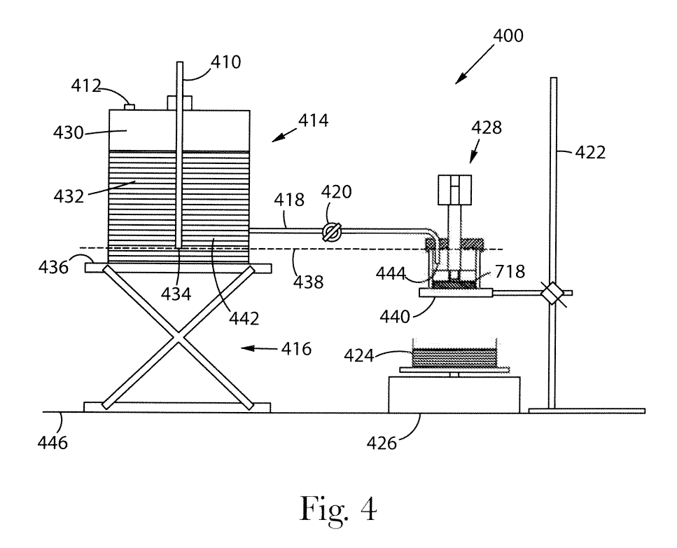

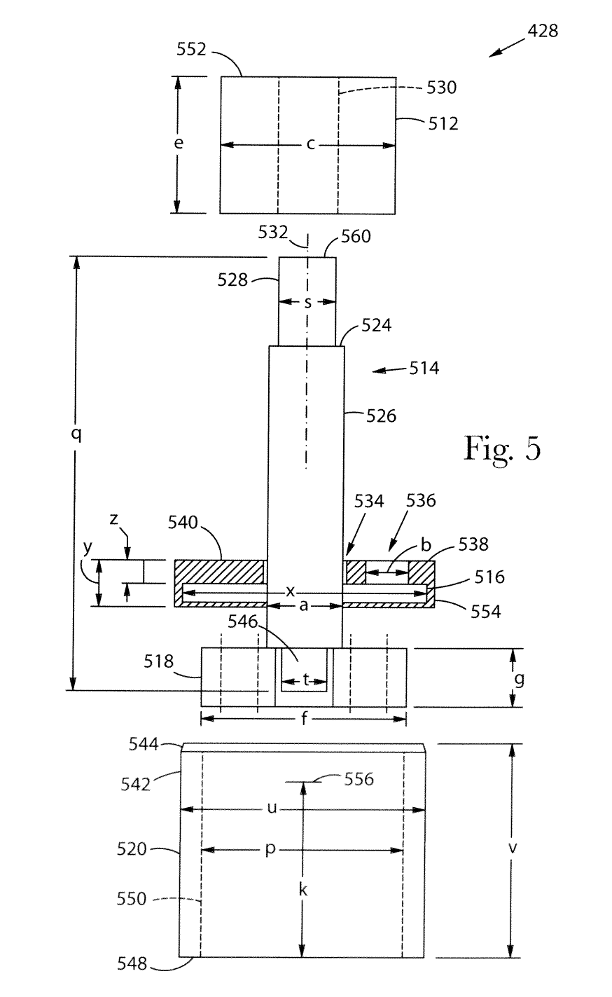

FIG. 5 shows the piston/cylinder assembly 428 comprising a metal weight 512, piston shaft 514, piston head 518, lid 516, and cylinder 520. The cylinder 520 is made of transparent polycarbonate (e.g., Lexan®) and has an inner diameter p of 6.00 cm (area=28.27 cm²) with inner cylinder walls 550 which are smooth. The bottom 548 of the cylinder 520 is faced with a US. Standard 400 mesh stainless-steel screen cloth (not shown) that is bi-axially stretched to tautness prior to attachment to the bottom 548 of the cylinder 520. The piston shaft 514 is made of transparent polycarbonate (e.g., Lexan®) and has an overall length q of approximately 127 mm. A middle portion 526 of the piston shaft 514 has a diameter r of 21.15 mm. An upper portion 528 of the piston shaft 514 has a diameter s of 15.8 mm, forming a shoulder 524. A lower portion 546 of the piston shaft 514 has a diameter t of approximately ⅝ inch and is threaded to screw firmly into the center hole 618 (see FIG. 6) of the piston head 518. The piston head 518 is perforated, made of transparent polycarbonate (e.g., Lexan®), and is also screened with a stretched US. Standard 400 mesh stainless-steel screen cloth (not shown). The weight 512 is stainless steel, has a center bore 530, slides onto the upper portion 528 of piston shaft 514 and rests on the shoulder 524. The combined weight of the piston head 518, piston shaft 514 and weight 512 is 596 g (±6 g), which corresponds to 0.30 psi over the area of the cylinder 520. The combined weight may be adjusted by drilling a blind hole down a central axis 532 of the piston shaft 514 to remove material and/or provide a cavity to add weight. The cylinder lid 516 has a first lid opening 534 in its center for vertically aligning the piston shaft 514 and a second lid opening 536 near the edge 538 for introducing fluid from the constant hydrostatic head reservoir 414 into the cylinder 520.

A first linear index mark (not shown) is scribed radially along the upper surface 552 of the weight 512, the first linear index mark being transverse to the central axis 532 of the piston shaft 514. A corresponding second linear index mark (not shown) is scribed radially along the top surface 560 of the piston shaft 514, the second linear index mark being transverse to the central axis 532 of the piston shaft 514. A corresponding third linear index mark (not shown) is scribed along the middle portion 526 of the piston shaft 514, the third linear index mark being parallel with the central axis 532 of the piston shaft 514. A corresponding fourth linear index mark (not shown) is scribed radially along the upper surface 540 of the cylinder lid 516, the fourth linear index mark being transverse to the central axis 532 of the piston shaft 514. Further, a corresponding fifth linear index mark (not shown) is scribed along a lip 554 of the cylinder lid 516, the fifth linear index mark being parallel with the central axis 532 of the piston shaft 514. A corresponding sixth linear index mark (not shown) is scribed along the outer cylinder wall 542, the sixth linear index mark being parallel with the central axis 532 of the piston shaft 514. Alignment of the first, second, third, fourth, fifth, and sixth linear index marks allows for the weight 512, piston shaft 514, cylinder lid 516, and cylinder 520 to be re-positioned with the same orientation relative to one another for each measurement.

The cylinder 520 specification details are:
Outer diameter u of the Cylinder 520: 70.35 mm
Inner diameter p of the Cylinder 520: 60.0 mm
Height v of the Cylinder 520: 60.5 mm

The cylinder lid 516 specification details are:
Outer diameter w of cylinder lid 516: 76.05 mm
Inner diameter x of cylinder lid 516: 70.5 mm
Thickness y of cylinder lid 516 including lip 554: 12.7 mm
Thickness z of cylinder lid 516 without lip: 6.35 mm
Diameter a of first lid opening 534: 22.25 mm
Diameter b of second lid opening 536: 12.7 mm
Distance between centers of first and second lid openings 534 and 536: 23.5 mm

The weight 512 specification details are:
Outer diameter c: 50.0 mm
Diameter d of center bore 530: 16.0 mm
Height e: 39.0 mm

The piston head 518 specification details are
Diameter f: 59.7 mm
Height g: 16.5 mm
Outer holes 614 (14 total) with a 9.65 mm diameter h, outer holes 614 equally spaced with centers being 47.8 mm from the center of center hole 618
Inner holes 616 (7 total) with a 9.65 mm diameter i, inner holes 616 equally spaced with centers being 26.7 mm from the center of center hole 618
Center hole 618 has a diameter j of ⅝ inches and is threaded to accept a lower portion 546 of piston shaft 514.

Prior to use, the stainless steel screens (not shown) of the piston head 518 and cylinder 520 should be inspected for clogging, holes or over-stretching and replaced when necessary. An SFC apparatus with damaged screen can deliver erroneous SFC results, and must not be used until the screen has been replaced.

A 5.00 cm mark 556 is scribed on the cylinder 520 at a height k of 5.00 cm (±0.05 cm) above the screen (not shown) attached to the bottom 548 of the cylinder 520. This marks the fluid level to be maintained during the analysis. Maintenance of correct and constant fluid level (hydrostatic pressure)is critical for measurement accuracy.

A constant hydrostatic head reservoir 414 is used to deliver salt solution 432 to the cylinder 520 and to maintain the level of salt solution 432 at a height k of 5.00 cm above the screen (not shown) attached to the bottom 548 of the cylinder 520. The bottom 434 of the air-intake tube 410 is positioned so as to maintain the salt solution 432 level in the cylinder 520 at the required 5.00 cm height k during the measurement, i.e., bottom 434 of the air tube 410 is in approximately same plane 438 as the 5.00 cm mark 556 on the cylinder 520 as it sits on the support screen (not shown) on the ring stand 440 above the receiving vessel 424. Proper height alignment of the air-intake tube 410 and the 5.00 cm mark 556 on the cylinder 520 is critical to the analysis. A suitable reservoir 414 consists of a jar 430 containing: a horizontally oriented L-shaped delivery tube 418 for fluid delivery, a vertically oriented open-ended tube 410 for admitting air at a fixed height within the constant hydrostatic head reservoir 414, and a stoppered vent 412 for re-filling the constant hydrostatic head reservoir 414. Tube 410 has an internal diameter of xx mm. The delivery tube 418, positioned near the bottom 442 of the constant hydrostatic head reservoir 414, contains a stopcock 420 for starting/stopping the delivery of salt solution 432. The outlet 444 of the delivery tube 418 is dimensioned to be inserted through the second lid opening 536 in the cylinder lid 516, with its end positioned below the surface of the salt solution 432 in the cylinder 520 (after the 5.00 cm height of the salt solution 432 is attained in the cylinder 520). The air-intake tube 410 is held in place with an o-ring collar (not shown). The constant hydrostatic head reservoir 414 can he positioned on a laboratory jack 416 in order to adjust its height relative to that of the cylinder 520. The components of the constant hydrostatic head reservoir 414 are sized so as to rapidly fill the cylinder 520 to the required height hydrostatic head) and maintain this height for the duration of the measurement. The constant hydrostatic head reservoir 414 must be capable of delivering salt solution 432 at a flow rate of at least 3 g/sec for at least 10 minutes,

The piston/cylinder assembly 428 is positioned on a 16 mesh rigid stainless steel support screen (not shown) (or equivalent) which is supported on a ring stand 440 or suitable alternative rigid stand. This support screen (not shown) is sufficiently permeable so as to not impede salt solution 432 flow and rigid enough to support the stainless steel mesh cloth (not shown) preventing stretching. The support screen (not shown) should be flat and level to avoid tilting the piston/cylinder assembly 428 during the test. The salt solution 432 passing through the support screen (not shown) is collected in a receiving vessel 424, positioned below (but not supporting) the support screen (not shown). The receiving vessel 424 is positioned on the balance 426 which is accurate to at least 0.01 g. The digital output of the balance 426 is connected to a computerized data acquisition system (not shown).

Preparation of Reagents (Not Illustrated)

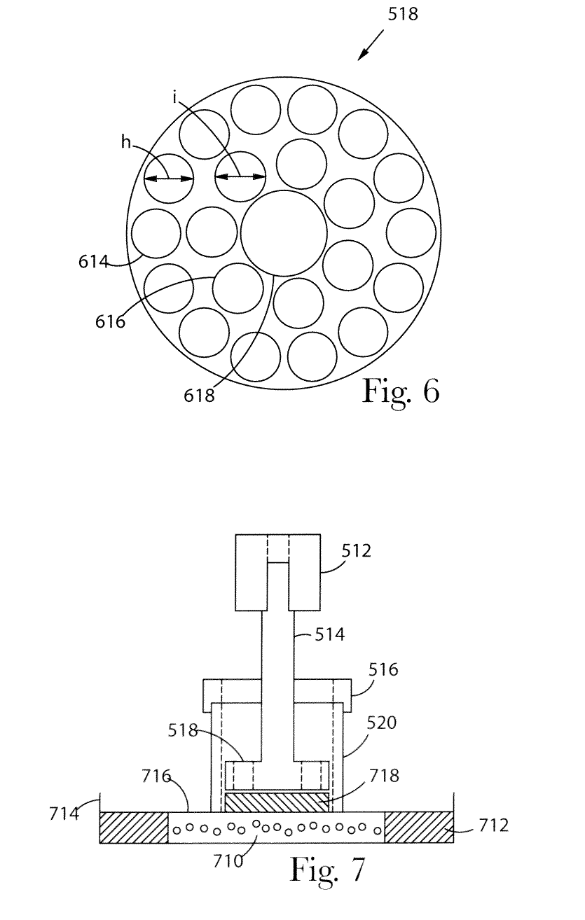

Jayco Synthetic Urine (JSU) 712 (see FIG. 7) is used for a swelling phase (see SFC Procedure below) and 0.118 M Sodium Chloride (NaCl) Solution is used for a flow phase (see SFC Procedure below). The following preparations are referred to a standard 1 liter volume. For preparation of volumes other than 1 liter, all quantities are scaled accordingly.

JSU: A 1L volumetric flask is filled with distilled water to 80% of its volume, and a magnetic stir bar is placed in the flask. Separately, using a weighing paper or beaker the following amounts of dry ingredients are weighed to within ±0.01 g using an analytical balance and are added quantitatively to the volumetric flask in the same order as listed below. The solution is stirred on a suitable stir plate until all the solids are dissolved, the stir bar is removed, and the solution diluted to 1L volume with distilled water. A stir bar is again inserted, and the solution stirred on a stirring plate for a few minutes more.

Quantities of salts to make 1 liter of Jayco Synthetic Urine:
Potassium Chloride (KCl) 2.00 g
Sodium Sulfate (Na₂SO4) 2.00 g
Ammonium dihydrogen phosphate (NH₄H₂PO₄) 0.85 g
Ammonium phosphate, dibasic ((NH₄)₂HPO₄) 0.15 g
Calcium Chloride (CaCl₂) 0.19 g—or hydrated calcium chloride (CaCl₂.2H₂O) 0.25 g]
Magnesium chloride (MgCl₂) 0.23 g [or hydrated magnesium chloride (MgCl₂.6H₂O) 0.50 g]

To make the preparation faster, each salt is completely dissolved before adding the next one. Jayco synthetic urine may be stored in a clean glass container for 2 weeks. The solution should not be used if it becomes cloudy. Shelf life in a clean plastic container is 10 days.

0.118 M Sodium Chloride (NaCl) Solution: 0.118 M Sodium Chloride is used as salt solution 432. Using a weighing paper or beaker 6.90 g (±0.01 g) of sodium chloride is weighed and quantitatively transferred into a IL volumetric flask; and the flask is filled to volume with distilled water. A stir bar is added and the solution is mixed on a stirring plate until all the solids are dissolved.

Test Preparation

Using a solid reference cylinder weight (not shown) (40 mm diameter; 140 mm height), a caliper gauge (not shown) (e.g., Mitotoyo Digimatic Height Gage) is set to read zero. This operation is conveniently performed on a smooth and level bench top 446. The piston/cylinder assembly 428 without superabsorbent is positioned under the caliper gauge (not shown) and a reading, L_{1,} is recorded to the nearest 0.01 mm.

The constant hydrostatic head reservoir 414 is filled with salt solution 432. The bottom 434 of the air-intake tube 410 is positioned so as to maintain the top part (not shown) of the liquid meniscus (not shown) in the cylinder 520 at the 5.00 cm mark 556 during the measurement. Proper height alignment of the air-intake tube 410 at the 5.00 cm mark 556 on the cylinder 520 is critical to the analysis.

The receiving vessel 424 is placed on the balance 426 and the digital output of the balance 426 is connected to a computerized data acquisition system (not shown). The ring stand 440 with a 16 mesh rigid stainless steel support screen (not shown) is positioned above the receiving vessel 424. The 16 mesh screen (not shown) should be sufficiently rigid to support the piston/cylinder assembly 428 during the measurement. The support screen (not shown) must be flat and level.

SFC Procedure

0.9 g (±0.05 g) of superabsorbent is weighed onto a suitable weighing paper using an analytical balance. 0.9 g (±0.05 g) of superabsorbent is weighed onto a suitable weighing paper using an analytical balance. The moisture content of the superabsorbent is measured according to the Edana Moisture Content Test Method 430.1-99 (“Superabsorbent materials—Polyacrylate superabsorbent powders—MOISTURE CONTENT—WEIGHT LOSS UPON HEATING” (February 99)). If the moisture content of the polymer is greater than 5%, then the polymer weight should be corrected for moisture (i.e., the added polymer should be 0.9 g on a dry-weight basis).

The empty cylinder 520 is placed on a level benchtop 446 and the superabsorbent is quantitatively transferred into the cylinder 520. The superabsorbent particles are evenly dispersed on the screen (not shown) attached to the bottom 548 of the cylinder 520 by gently shaking, rotating, and/or tapping the cylinder 520. It is important to have an even distribution of particles on the screen (not shown) attached to the bottom 548 of the cylinder 520 to obtain the highest precision result. After the superabsorbent has been evenly distributed on the screen (not shown) attached to the bottom 548 of the cylinder 520 particles must not adhere to the inner cylinder walls 550. The piston shaft 514 is inserted through the first lid opening 534, with the lip 554 of the lid 516 facing towards the piston head 518. The piston head 518 is carefully inserted into the cylinder 520 to a depth of a few centimeters. The lid 516 is then placed onto the upper rim 544 of the cylinder 520 while taking care to keep the piston head 518 away from the superabsorbent. The lid 516 and piston shaft 526 are then carefully rotated so as to align the third, fourth, fifth, and sixth linear index marks are then aligned. The piston head 518 (via the piston shaft 514) is then gently lowered to rest on the dry superabsorbent. The weight 512 is positioned on the upper portion 528 of the piston shaft 514 so that it rests on the shoulder 524 such that the first and second linear index marks are aligned. Proper seating of the lid 516 prevents binding and assures an even distribution of the weight on the hydrogel layer 718.

Swelling Phase: An 8 cm diameter fritted disc (7 mm thick; e.g. Chemglass Inc. #CG 201-51, coarse porosity) 710 is saturated by adding excess JSU 712 to the fritted disc 710 until the fritted disc 710 is saturated. The saturated fritted disc 710 is placed in a wide flat-bottomed Petri dish 714 and JSU 712 is added until it reaches the top surface 716 of the fritted disc 710. The JSU height must not exceed the height of the fitted disc 710.

The screen (not shown) attached to the bottom 548 of the cylinder 520 is easily stretched. To prevent stretching, a sideways pressure is applied on the piston shaft 514, just above the lid 516, with the index finger while grasping the cylinder 520 of the piston/cylinder assembly 428. This “locks” the piston shaft 514 in place against the lid 516 so that the piston/cylinder assembly 428 can be lifted without undue force being exerted on the screen (not shown).

The entire piston/cylinder assembly 428 is lifted in this fashion and placed on the fritted disc 710 in the Petri dish 714. JSU 712 from the Petri dish 714 passes through the fritted disc 710 and is absorbed by the superahsorhent polymer (not shown) to form a hydrogel layer 718. The JSU 712 available in the Petri dish 714 should be enough for all the swelling phase. If needed, more JSU 712 may be added to the Petri dish 714 during the hydration period to keep the JSU 712 level at the top surface 716 of the fritted disc 710. After a period of 60 minutes, the piston/cylinder assembly 428 is removed from the fritted disc 710, taking care to lock the piston shaft 514 against the lid 516 as described above and ensure the hydrogel layer 718 does not lose JSU 712 or take in air during this procedure. The piston/cylinder assembly 428 is placed under the caliper gauge (not shown) and a reading, L₂, is recorded to the nearest 0.01 mm. If the reading changes with time, only the initial value is recorded. The thickness of the hydrogel layer 718. L₀ is determined from L₂−L₁ to the nearest 0.1 mm.

The entire piston/cylinder assembly 428 is lifted in this the fashion described above and placed on the support screen (not shown) attached to the ring stand 440. Care should be taken so that the hydrogel layer 718 does not lose JSU 712 or take in air during this procedure. The JSU 712 available in the Petri dish 714 should be enough for all the swelling phase. If needed, more JSU 712 may be added to the Petri dish 714 during the hydration period to keep the JSU 712 level at the 5.00 cm mark 556. After a period of 60 minutes, the piston/cylinder assembly 428 is removed, taking care to lock the piston shaft 514 against the lid 516 as described above. The piston/cylinder assembly 428 is placed under the caliper gauge (not shown) and the caliper (not shown) is measured as L₂ to the nearest 0.01 mm. The thickness of the hydrogel layer 718, L₀ is determined from L₂−L₁ to the nearest 0.1 mm. If the reading changes with time, only the initial value is recorded.

The piston/cylinder assembly 428 is transferred to the support screen (not shown) attached to the ring support stand 440 taking care to lock the piston shaft 514 in place against the lid 516. The constant hydrostatic head reservoir 414 is positioned such that the delivery tube 418 is placed through the second lid opening 536. The measurement is initiated in the following sequence:
a) The stopcock 420 of the constant hydrostatic head reservoir 410 is opened to permit the salt solution 432 to reach the 5.00 cm mark 556 on the cylinder 520. This salt solution 432 level should be obtained within 10 seconds of opening the stopcock 420,
b) Once 5.00 cm of salt solution 432 is attained, the data collection program is initiated,
   With the aid of a computer (not shown) attached to the balance 426, the quantity of salt solution 432 passing through the hydrogel layer 718 is recorded at intervals of 20 seconds for a time period of 10 minutes. At the end of 10 minutes, the stopcock 420 on the constant hydrostatic head reservoir 410 is closed. The piston/cylinder assembly 428 is removed immediately, placed under the caliper gauge (not shown) and a reading, L₃, is recorded to the nearest 0.01 mm. The final thickness of the hydrogel layer 718. L_{f} is determined from L₃−L₁ to the nearest 0.1 mm, as described above. The percent change in thickness of the hydrogel layer 718 is determined from (L_{f}/L₀)×100. Generally the change in thickness of the hydrogel layer 718 is within about ±10%.

The data from 60 seconds to the end of the experiment are used in the SFC calculation. The data collected prior to 60 seconds are not included in the calculation. The flow rate Fₛ (in g/s) is the slope of a linear least-squares fit to a graph of the weight of salt solution 432 collected (in grams) as a function of time (in seconds) from 60 seconds to 600 seconds.

In a separate measurement, the flow rate through the permeability measurement system 400 (Fₐ) is measured as described above, except that no hydrogel layer 718 is present. If Fₐ is much greater than the flow rate through the permeability measurement system 400 when the hydrogel layer 718 is present, Fₛ, then no correction for the flow resistance of the permeability measurement system 400 (including the piston/cylinder assembly 428) is necessary. In this limit, F_{g}=Fₛ, where F_{g} is the contribution of the hydrogel layer 718 to the flow rate of the permeability measurement system 400. However if this requirement is not satisfied, then the following correction is used to calculate the value of F_{g} from the values of Fₛ and Fₐ:

F_{g}=(Fₐ×Fₛ)/(Fₐ−Fₛ)

The Saline Flow Conductivity (K) of the hydrogel layer 718 is calculated using the following equation:

K=[F_{g}(t=0)×L₀]/[ρ×A×ΔP],

where F_{g} is the flow rate in g/sec determined from regression analysis of the flow rate results and any correction due to permeability measurement system 400 flow resistance, L₀ is the initial thickness of the hydrogel layer 718 in cm, ρ is the density of the salt solution 432 in gm/cm³. A (from the equation above) is the area of the hydrogel layer 718 in cm², ΔP is the hydrostatic pressure in dyne/cm², and the saline flow conductivity, K, is in units of cm³ sec/gm. The average of three determinations should be reported.

For hydrogel layers 718 where the flow rate is substantially constant, a permeability coefficient (κ) can be calculated from the saline flow conductivity using the following equation:

κ=K η

where η is the viscosity of the salt solution 432 in poise and the permeability coefficient, κ, is in units of cm².

In general, flow rate need not be constant. The time-dependent flow rate through the system, F_{S} (t) is determined, in units of g/sec, by dividing the incremental weight of salt solution 432 passing through the permeability measurement system 400 (in grams) by incremental time (in seconds). Only data collected for times between 60 seconds and 10 minutes is used for flow rate calculations. Flow rate results between 60 seconds and 10 minutes are used to calculate a value for Fₛ (t=0), the initial flow rate through the hydrogel layer 718. Fₛ (t=0) is calculated by extrapolating the results of a least-squares fit of F_{S} (t) versus time to t=0.

### Absorption Against Pressure

This test measures the amount of a 0.90% saline solution absorbed by superabsorbent polymers that are laterally confined in a piston/cylinder assembly under a confining pressure for a period of one hour. European Disposables and Nonwovens Association (EDANA) test method 442.2-02 entitled “Absorption Under Pressure” is used.

### Basis Weight

This test measures the mass per unit area for a substrate. European Disposables and Nonwovens Association (EDANA) test method 40.3-90 entitled “Mass Per Unit Area” is used.

### Liquid Strike-Through

This test measures the time it takes for a known volume of liquid applied to the surface of a substrate to pass through the substrate to an underlying absorbent pad. European Disposables and Nonwovens Association (EDANA) test method 150.4-99 entitled “Liquid Strike-Through Time” is used.

### Tensile Test

This test measures the peak load exhibited by a substrate. A preferred piece of equipment to do the test is a tensile tester such as a MTS Synergie 100 or a MTS Alliance, fitted with a computer interface and Testworks 4 software, available from MTS Systems Corporation 14000 Technology Drive, Eden Prairie, Minn., USA. This instrument measures the Constant Rate of Extension in which the pulling grip moves at a uniform rate and the force measuring mechanism moves a negligible distance (less than 0.13 mm) with increasing force. The load cell is selected such that the measured loads (e.g., force) of the tested samples will be between 10 and 90% of the capacity of the load cell (typically a 25N or 50N load cell).

A 1×1 inch (2.5×2.5 cm) sample is die-cut from the substrate using an anvil hydraulic press die to cut the film with the die into individual samples. A minimum of three samples are created which are substantially free of visible defects such as air bubbles, holes, inclusions, and cuts. Each sample must have smooth and substantially defect-free edges. Testing is performed in a conditioned room having a temperature of 23° C. (±1° C.) and a relative humidity of 50% (±2%) for at least 2 hours. Samples are allowed to equilibrate in the conditioned room for at least 2 hours prior to testing.

Pneumatic jaws of the tensile tester, fitted with flat 2.54 cm-square rubber-faced grips, are set to give a gauge length of 2.54 cm. The sample is loaded with sufficient tension to eliminate observable slack, but less than 0.05N. The sample is extended at a constant crosshead speed of 25.4 cm/min until the specimen completely breaks. If the sample breaks at the grip interface or slippage within the grips is detected, then the data is disregarded and the test is repeated with a new sample and the grip pressure is appropriately adjusted. Samples are run at least in triplicate to account for film variability.

The resulting tensile force-displacement data are converted to stress-strain curves. Peak load is defined as the maximum stress measured as a specimen is taken to break, and is reported in Newtons per centimeter width (as measured parallel to the grips) of the sample. The peak load for a given substrate is the average of the respective values of each sample from the substrate.

### Moisture Vapor Transmission Rate (MVTR) Test

The MVTR test method measures the amount of water vapor that is transmitted through a film under specific temperature and humidity. The transmitted vapor is absorbed by CaCl2 desiccant and determined gravimetrically. Samples are evaluated in triplicate, along with a reference film sample of established permeability (e.g., Exxon Exxaire microporous material #XBF-110W) that is used as a positive control.

This test uses a flanged cup machined from Delrin (McMaster-Carr Catalog #8572K34) and anhydrous CaCl2 (Wako Pure Chemical Industries, Richmond, Va.; Catalog 030-00525).

The height of the cup is 55 mm with an inner diameter of 30 mm and an outer diameter of 45 mm. The cup is fitted with a silicone gasket and lid containing 3 holes for thumb screws to completely seal the cup.

The cup is filled with CaCl, to within 1 cm of the top. The cup is tapped on the counter 10 times, and the CaCl₂ surface is leveled. The amount of CaCl₂ is adjusted until the headspace between the film surface and the top of the CaCl2 is 1.0 cm. The film is placed on top of the cup across the opening (30 mm) and is secured using the silicone gasket, retaining ring, and thumb screws. Properly installed, the specimen should not be wrinkled or stretched.

The film must completely cover the cup opening, A, which is 0.0007065 m².

The sample assembly is weighed with an analytical balance and recorded to ±0.001 g. The assembly is placed in a constant temperature (40±3° (−) and humidity (75±3% RH) chamber for 5.0 hr ±5 min. The sample assembly is removed, covered with Saran Wrap® and is secured with a rubber band. The sample is equilibrated to room temperature for 30 min, the plastic wrap removed, and the assembly is reweighed and the weight is recorded to ±0.001 g. The absorbed moisture Mₐ is the difference in initial and final assembly weights. MVTR, in g/m²/24 hr (g/m²/24 hours), is calculated as:

$$MVTR = \frac{( {M}_{a} \times 24 )}{( A \times 5 hours )}$$

Replicate results are averaged and rounded to the nearest 100 g/m²/24 hr, e.g., 2865 g/m²/24 hours is herein given as 2900 g/m²/24 hours and 275 g/m²/24 hours is given as 300 g/m²/24 hours

The Hydrohead test method measures the resistance of substrates (e.g., particularly nonwovens) to the penetration of water. World Strategic Partners (WSP) test method 80.6 (05) entitled “Standard Test Method for Evaluation of Water Resistance (Hydrostatic Pressure) Test” is used. WSP methods are harmonized test methods formulated by EDANA and the Association of the Nonwoven Fabrics Industry (INDA). The test is to be run with an incoming water supply rate of 10±0.5 cm water/minute.

### XI. EXAMPLES

### Example 1

### Polyolefin

A suitable polyolefin may be created according to the following method. An exemplary renewable resource is corn. The corn is cleaned and may be degerminated. The corn is milled to produce a fine powder (e.g., cornmeal) suitable for enzymatic treatment. The hydrolysis (e.g., liquification and saccharification) of the corn feedstock to yield fermentable sugars is well known in the agricultural and biofermentation arts. A suitable preparation pathway is disclosed in U.S. Pat. No. 4,407,955. A slurry of dry milled corn is created by adding water to the milled corn and an aqueous solution of sulfuric acid (98% acid by weight). Sufficient sulfuric acid should be added to provide a slurry pH of about 1.0 to about 2.5. The slurry is heated to about 140° C. to about 220° C. and pressurized to at least about 50 psig; however, pressures from about 100 psig to about 1,000 psig may result in greater conversion of the starch to fermentable sugars. The slurry is maintained at the aforementioned temperature and pressure for a few seconds up to about 10 minutes. The slurry may be conveyed through one or more pressure reduction vessels which reduce the pressure and temperature of hydrolyzed slurry. The slurry is subjected to standard separation techniques such as by centrifuge to yield a fermentable sugar liquor. The liquor typically has a dextrose equivalent of at least 75. The resulting sugar liquor is fermented according to processes well know to a skilled artisan using a suitable strain of yeast (e.g., genus of Saccharomyces). The resulting ethanol may be separated from the aqueous solution by standard isolation techniques such as evaporation or distillation.

Ethanol is dehydrated to form ethylene by heating the ethanol with an excess of concentrated sulfuric acid to a temperature of about 170° C. Ethylene may also be formed by passing ethanol vapor over heated aluminum oxide powder.

The resulting ethylene is polymerized using any of the well known polymerization techniques such as free radical polymerization, Ziegler-Natta polymerization, or metallocene catalyst polymerization. Low density branched polyethylene (LDPE) is often made by free radical vinyl polymerization. Linear low density polyethylene (LLDPE) is made by a more complicated procedure called Ziegler-Natta polymerization. The resulting polyethylene or blends thereof may be processed to yield a desired end product such as a film, fiber, or filament.

As an example, a linear low density polyethylene is made by copolymerizing ethylene with other longer chain olefins to result in a polymer having a density of about 0.915 g/cm³ to about 0.925 g/cm³. A 49 grams/meter² (gsm) cast extruded film is made comprising the linear low density polyethylene and about 35% by weight to about 45% by weight calcium carbonate (available from English China Clay of America. Inc. under the designation Supercoat™). The film may be made porous via several routes. The film may be warmed and elongated to 500% of the film's original length using well known elongation methods and machinery. The resulting microporous film is capable of exhibiting a MVTR of at least 2000 g/m²/24 hours. Alternately, the film may be incrementally stretched according to the method disclosed in U.S. Pat. No. 6,605,172. The resulting microporous film should exhibit a MVTR of at least 2000 g/m²/24 hours.

A nonwoven spunbond web may be formed according to methods well known in the art such as evidenced by U.S. Pat. Nos. 4,405,297 and 4,340,563. The web is formed to have a basis weight of about 5 gsm to about 35 gsm. The individual filaments can have an average denier of about 5 or less. The individual filaments may have a variety of cross-sectional shapes. A suitable cross-sectional shape is a bilobal shape disclosed in U.S. Pat. No. 4,753,834. The resultant nonwoven may be made more hydrophilic by incorporating a surfactant in the nonwoven as described in U.S. Statutory Invention Registration No. H1670. The nonwoven treated to be more hydrophilic is suitable for use as a topsheet in an absorbent article. The nonwoven should exhibit a Liquid Strike-Through Time of less than about 4 seconds. The resultant nonwoven may be made more hydrophobic by use of a surface coating as described in U.S. Publication No. 2005/0177123A1. The nonwoven treated to be more hydrophobic is suitable for use a cuff substrate in an absorbent article. The treated nonwoven should exhibit a hydrohead of at least about 5 mbar.

### EXAMPLE 2

### Superabsorbent Polymer

Preparation of Glycerol

Canola oil is obtained by expressing from canola seeds. Approximately 27.5 kg of canola oil, 5.3 kg methanol and 400 g sodium methoxide are charged to a 50 L round-bottomed flask equipped with a heating mantle, thermometer, nitrogen inlet, mechanical stirrer, and reflux condenser. A glass eduction tube (dip tube) is situated so that liquid can be removed from the bottom of the flask by means of a peristaltic pump. The flask is purged with nitrogen and the mixture in the flask is heated to 65° C. with stirring. The mixture is allowed to reflux for 2.5 hours, then the heat is turned off, agitation is stopped and the mixture allowed to settle for 20 minutes. The bottom layer is pumped out of the flask and kept for further use (Fraction 1). Approximately 1.4 kg methanol and 230 g sodium methoxide are added to the flask, agitation is resumed, and the mixture refluxed at 65° C. for another 2 hours. The heat is turned off, approximately 2.8 L of water are added to the flask and the mixture is stirred for 1 minute. The stirrer is turned off and the mixture allowed to settle for 20 minutes. The bottom layer is then pumped out of the flask and kept for further use (Fraction 2). Approximately 1.6 L of water is added to the flask, and the mixture is stirred for 1 minute. The stirrer is turned off and the mixture allowed to settle for 20 minutes. The bottom layer is then pumped out of the flask and kept for further use (Fraction 3). Fractions 1, 2 and 3 are combined in a suitable flask equipped with a magnetic stirrer. The combined fractions are stirred to form a homogeneous mixture and heated to 82° C. Sodium hydroxide solution (50%) is added slowly until the pH of the mixture is 11-13 and the temperature is maintained at 82° C. for a further 10 minutes. The pH is checked and more NaOH solution added if <11. The solution is concentrated at 115° C. under a vacuum of approximately 40 mm Hg until bubbling ceases (water content<5%). The solution is transferred to a round bottomed flask and the glycerol is vacuum distilled using a rotary evaporator with the oil bath temperature at 170° C. and the condenser at 130-140° C. The vacuum is controlled to achieve a moderate distillation rate. A center cut of distilled glycerol is collected.

### Preparation of Acrolein

Approximately 200 g of fused aluminum oxide, 6-12 US standard mesh, primarily α-phase, is mixed with 50 g of a 20% solution of phosphoric acid for one hour. The mixture is dried under vacuum by means of a rotary evaporator with the oil bath temperature at 80° C. A stainless steel tube (chromatography column) with an internal diameter of approximately 15 mm and contour length approximately 60 cm is packed with the dried particles. The column is installed in a gas chromatogram instrument with the inlet connected to the injector port, and the outlet connected to a condenser and collection vessel. The column and injector port are heated to 300° C. and a 20% aqueous solution of glycerol derived from canola oil is injected at a rate of 40 mL/h. An inert carrier gas such as helium is optionally utilized to help transport the vapor through the column. The vapors emanating from the column outlet are condensed and collected. Acrolein is isolated from the condensate by fractional distillation or other suitable methods known to those skilled in the art.

Preparation of Acrylic Acid

A Pyrex glass reactor approximately 12 cm×2.5 cm OD equipped with a thermowell is packed with 31 g (30 mL bulk volume) of a catalyst containing 2 wt % palladium and 0.5 wt % copper supported on alumina. The reactor is heated in an oil bath at 152° C. A gaseous stream consisting of 3.4% acrolien, 14.8% oxygen, 22.9% steam, and 58.5% nitrogen by volume, is passed through the heated catalyst at such urate that the superficial contact time was about 5 seconds. The reaction mixture is then passed through two water scrubbers connected in series held at 0° C. The aqueous solutions collected are combined and acrylic acid separated from the mixture by fractional distillation.

Preparation of Superabsorbent Polymer

L-Ascorbic Acid (0.2081 g, 1.18 mmol) is added to a 100 mL volumetric flask and is dissolved in distilled water (approximately 50 mL). After approximately ten minutes the solution is diluted to the 100 mL mark on the volumetric flask with distilled water and the flask was inverted and agitated to ensure a homogeneous solution.

To a 3L jacketed resin kettle is added TMPTA (0.261 g, 0.881 mmol), acrylic acid (296.40 g, 4.11 mol), and distilled water (250 g). Water is circulated through the jacket of the resin kettle by means of a circulating water bath kept at 25° C. To the monomer solution is added standard 5N sodium hydroxide solution (576 mL, 2.88 mol). The resin kettle is capped with a lid having several ports. An overhead mechanical stirrer is set up using an air-tight bushing in the central port. A thermometer is inserted through a seal in another port so that the bulb of the thermometer is immersed in the mixture throughout the reaction. The solution is stirred using the overhead mechanical stirrer and purged with nitrogen using a fritted gas dispersion tube for approximately fifteen minutes. Nitrogen is vented from the kettle via an 18-gauge syringe needle inserted through a septum in the lid.

After approximately fifteen minutes the fritted gas dispersion tube is raised above the surface of the monomer solution and nitrogen was kept flowing through the headspace of the kettle. A solution of sodium persulfate (0.4906 g, 2.06 mmol) in distilled water (5 mL), and then a small aliquot of the L-ascorbic acid solution (1 mL, 1.18 mmol) is added via syringe. The mechanical stirrer is stopped when the vortex in the polymer solution disappears due to the increase in viscosity of the solution (a few seconds after adding the L-ascorbic acid solution). The polymerization reaction proceeds with the circulating bath at 25° C. for 30 minutes. After 30 minutes the temperature of the water bath is increased to 40° C. and held for an additional 30 minutes. The temperature of the water bath is then increased to 50° C. and held for another hour. The peak temperature of the static polymerization is approximately 70° C.

After one hour at 50° C. the circulating water bath is turned off. The resin kettle is opened; the polyacrylate gel is removed and broken into chunks approximately 2 cm in diameter. These are chopped into smaller particles using a food grinder attachment with 4.6 mm holes on a

Kitchen-Aid mixer (Proline Model KSM5). Distilled water is added periodically from a squirt bottle to the infeed portion of the grinder to facilitate passage of the bulk gel through the grinder. Approximately 200 g of distilled water is used for this purpose. The chopped gel is spread into thin layers on two separate polyester mesh screens each measuring approximately 56 cm×48 cm and dried at 150° C. for 90 minutes in a vented oven in a fashion which allows passage of air through the mesh.

The dried gel is then milled through a Laboratory Wiley Mill using a 20-mesh screen. Care is taken to ensure that the screen does not become clogged during the grinding process. The milled dried gel is sieved to obtain a fraction with particles which pass through a No. 20 USA Standard Testing Sieve and are retained on a No. 270 USA Standard Testing Sieve. The ‘on 20’ and ‘through 270’ fractions are discarded.

The resultant free-flowing powder fraction ‘through 20’ and ‘on 270’ is dried under vacuum at room temperature until further use.

A 50% solution of ethylene carbonate (1,3-dioxolan-2-one) is prepared by dissolving 10.0 grams of ethylene carbonate in 10.0 grams of distilled water.

100.00 grams of the dried ‘through 20’ and ‘on 270’ powder above are added to a stainless steel mixing bowl (approximately 4L) of a Kitchen Aid mixer (Proline Model KSM5) equipped with a stainless steel wire whisk. The height of the mixing bowl is adjusted until the wire whisk just contacts the bowl. The whisk is started and adjusted to a speed setting of ‘6’ to stir the particles. Immediately thereafter, 15 grams of the above 50 wt % ethylene carbonate solution is added to the stirred AGM via a 10 mL plastic syringe equipped with a four inch 22-gauge needle. The solution is added directly onto the stirred particles over a period of several seconds. The syringe is weighed before and after the addition of solution to determine the amount added to the particles. After the solution is added, the mixture is stirred for approximately thirty seconds to help ensure an even coating. The resultant mixture is quite homogeneous with no obvious large clumps of material or residual dry powder. The mixture is then immediately transferred to a Teflon lined 20 cm×35 cm metal tray, spread into a thin layer and placed into a vented oven at 185° C. for one hour.

After one hour, the mixture is removed from the oven and allowed to cool for approximately one minute. After cooling the powder is placed in a 12 cm diameter mortar and any agglomerated pieces are gently broken apart with a pestle. The resultant powder is sieved to obtain a fraction which passes through a No. 20 US standard screen, but is retained on a No. 270 US standard screen.

The resultant ‘through 20’ and ‘on 270’ superabsorbent polymer particles are stored under vacuum at room temperature until further use. The AAP value for this material is measured according to the EDANA test method 442.2-02, and the SFC value is measured according to the SFC Test Method described above. The AAP value is found to be about 21 g/g, and the SFC value is found to be about 50×10⁻⁷ cm³·sec/g

All documents cited in the Detailed Description of the Invention are, in relevant part, incorporated herein by reference: the citation of any document is not to be construed as an admission that it is prior art with respect to the invention. To the extent that any definition or meaning of a term in this written document conflicts with any definition or meaning of the term in a document incorporated by reference, the definition or meaning, assigned to the term in this document shall govern.

While particular embodiments of the invention have been illustrated and described, it would be obvious to those skilled in the art that various other changes and modifications can he made without departing from the spirit and scope of the invention. It should be apparent that combinations of such embodiments and features are possible and can result in executions within the scope of this invention. It is therefore intended to cover in the appended claims all such changes and modifications that are within the scope of this invention.

What is claimed is:

1. An absorbent core for an absorbent article, the absorbent core (a) having a bio-based content of at least about 50% based on the total weight of the absorbent core and (b) comprising a synthetic polymer derived from a renewable resource via a first intermediate compound selected from the group consisting of crotonic acid, propiolactone, ethylene oxide, carbon monoxide. carbon dioxide, i-propanol, butanol, butyric acid, propionic acid, methyl lactate, ethyl lactate, 2-acetoxypropanoic acid, methyl 2-acetoxypropanoate, polyhydroxybutyrate, and a polyhydroxyalkanoate comprising 3-hydroxypropionate monomers.

2. The absorbent core of claim 1, wherein the first intermediate is crotonic acid, propiolactone or ethylene oxide.

3. The absorbent core of claim 2, wherein the synthetic polymer is derived from the renewable resource via acrylic acid as a second intermediate compound.

4. The absorbent core of claim 1, wherein the first intermediate is i-propanol, butanol, or propionic acid.

5. The absorbent core of claim 4, wherein the synthetic polymer is derived from the renewable resource via propylene as a second intermediate compound.

6. The absorbent core of claim 1, wherein the first intermediate is 2-acetoxypropanoic acid, methyl 2-acetoxypropanoate, methyl lactate, ethyl lactate, or a polyhydroxyalkanoate comprising 3-hydroxypropionate monomers.

7. The absorbent core of claim 1, wherein the first intermediate (a) is crotonic acid, ethylene, i-propanol, butanol, or propionic acid, and (b) is derived from a sugar via biofermentation.

8. The absorbent core of claim 1, wherein the synthetic polymer is a superabsorbent.

9. The absorbent core of claim 1, wherein the absorbent core comprises a bio-based content of at least about 65% based on the total weight of the absorbent core.

10. The absorbent core of claim 1, wherein the absorbent core comprises a bio-based content of at least about 80% based on the total weight of the absorbent core.

11. An absorbent article comprising the absorbent core of claim 1.

12. An absorbent core for an absorbent article, the absorbent core (a) having a bio-based content of at least about 70% based on the total weight of the absorbent core and (b) comprising a synthetic polymer derived from a renewable resource via a first intermediate compound selected from the group consisting of ethylene, n-propanol, and propylene.

13. An absorbent article comprising the absorbent core of claim 12,

14. An element of an absorbent article selected from the group consisting of a topsheet, a backsheet, a dusting layer, a fastener, and a barrier leg cuff, the element (a) having a bio-based content of at least about 50% based on the total weight of the element and (b) comprising a synthetic polymer derived from a renewable resource via a first intermediate compound selected from the group consisting of polyhydroxybutyrate, crotonic acid, i-propanol, and butanol.

15. The element of claim 14, wherein the first intermediate is i-propanol or butanol.

16. The element of claim 14, wherein the first intermediate is polyhydroxybutyrate or crotonic acid.

17. The element of claim 14, wherein the first intermediate (a) is crotonic acid, i-propanol, or butanol, and (b) is derived from a sugar via biofermentation.

18. The element of claim 14, wherein the element comprises a bio-based content of at least about 65% based on the total weight of the element.

19. The element of claim 14, wherein the element comprises a bio-based content of at least about 80% based on the total weight of the element.

20. An element for an absorbent article selected from the group consisting of a topsheet, a backsheet, a dusting layer, a fastener, and a barrier leg cuff, the element (a) having a bio-based content of at least about 70% based on the total weight of the element and (b) comprising a synthetic polymer derived from a renewable resource via a first intermediate compound selected from the group consisting of ethylene, n-propanol, and propylene.

21. An absorbent article comprising the absorbent core of claim 20.

22. A method for making an element for an absorbent article, the method comprising:
(a) converting an intermediate to acrylic acid or propylene, wherein the intermediate is derived from biomass and is selected from the group consisting of crotonic acid, propiolactone, ethylene oxide, i-propanol, butanol, 2-acetoxypropanoic acid, methyl 2-acetoxypropanoate, methyl lactate, ethyl lactate, polyhydroxybutyrate, and a polyhydroxyalkanoate comprising 3-hydroxypropionate monomers;
(b) polymerizing the acrylic acid or propylene to form a synthetic polymer; and
(c) disposing or incorporating the synthetic polymer into the element.

23. The method of claim 22, wherein the method further comprises (d) incorporating the element into an absorbent article.

24. The method of claim 22, wherein step (a) comprises converting propylene to acrylic acid.

25. The method of claim 22, wherein the intermediate is i-propanol, and step a comprises converting i-propanol to propylene via dehydration.

26. The method of claim 22, wherein the intermediate is butanol, and step (a) comprises converting butanol to butene via dehydration and converting butene to propylene via metathesis.

27. The method of claim wherein the intermediate is polyhydroxybutyrate, and step(a) comprises converting polyhydroxybutyrate to crotonic acid via thermolysis and converting the crotonic acid to propylene via decarboxylation.

28. The method of claim 22, wherein the intermediate is ethylene oxide, and step (a) comprises converting ethylene oxide to propiolactone via carbonylation, and converting the propiolactone to acrylic acid.

29. The method of claim 22, wherein the intermediate is crotonic acid, and step a comprises converting crotonic acid to acrylic acid via metathesis.

30. The method of claim 22, wherein the intermediate is crotonic acid, and step a comprises converting crotonic acid to propylene via metathesis.

31. The method of claim 22, wherein the intermediate is a polyhydroxyalkanoate comprising 3-hydroxypropionate monomers, a polyhydroxybutyrate, or a blend of polyhydroxyalkanoate comprising 3-hydroxypropionate monomers and a polyhydroxybutyrate, and step (a) comprises
(i) converting the polyhydroxyalkanoate comprising 3-hydroxypropionate monomers, the polyhydroxybutyrate, or the blend of polyhydroxyalkanoate comprising 3-hydroxypropionate monomers and a polyhydroxybutyrate to crotonic acid via thermolysis and converting the crotonic acid to acrylic acid via metathesis, or
(ii) converting the polyhydroxyalkanoate comprising 3-hydroxypropionate monomers or the blend of polyhydroxyalkanoate comprising 3-hydroxypropionate monomers and a polyhydroxybutyrate to acrylic acid via thermolysis.

32. The method of claim 22, wherein the intermediate is propionic acid, and step (a) comprises converting propionic acid to acrylic acid via dehydrogenation.

33. The method of claim 22, wherein the element is an absorbent core.

34. A method for making an element for an absorbent article, the method comprising:
(a) converting ethylene to acrylic acid via reaction with carbon dioxide;
(b) polymerizing the acrylic acid to form a synthetic polymer; and
(c) disposing or incorporating the synthetic polymer into the element.
